(19) United States
(12) Patent Application Publication
(10) Pub. No.: US 2018/0057465 A1
(43) Pub. Date: Mar. 1, 2018

(54) Inhibitors of Necroptosis

(71) Applicant: Catalyst Therapeutics Pty Ltd, Parkville (AU)

(72) Inventors: Guillaume Laurent Lessene, Bundoora (AU); Jean-Marc Garnier, Albert Park (AU); Anthony Nicholas Cuzzupe, St Albans (AU); John Thomas Feutrill, Rosanna (AU); Peter Edward Czabotar, North Melbourne (AU); Pooja Sharma, Truganina (AU)

(21) Appl. No.: 15/549,751

(22) PCT Filed: Feb. 10, 2016
(86) PCT No.: PCT/AU2016/050081

§ 371 Date: Aug. 9, 2017

(30) Foreign Application Priority Data
Feb. 10, 2015 (AU) 2015900416

(51) Int. Cl. C07D 239/48; C07D 403/12; C07D 491/107

(52) U.S. Cl. CPC C07D 239/48; C07D 491/107; C07D 403/12

(57) ABSTRACT

The invention relates to novel heterocyclic compounds of Formula (I) which inhibit necroptosis and methods for their use. The compounds may be useful in the treatment of conditions associated with deregulated necroptosis.

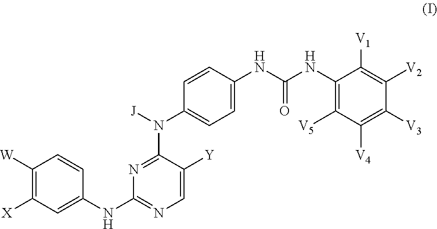

### FIELD OF THE INVENTION

The present disclosure relates to novel heterocyclic compounds which inhibit necroptosis and methods for their use.

### BACKGROUND OF THE INVENTION

In many diseases, cell death is mediated through apoptotic and/or necrotic pathways. While much is known about the mechanisms of action that control apoptosis, control of necrosis is not as well understood. Understanding the mechanisms in respect of both necrosis and apoptosis in cells is essential to being able to treat conditions, such as neurodegenerative diseases, stroke-coronary heart disease, kidney disease, liver disease, AIDS and the conditions associated with AIDS.

Cell death has traditionally been categorized as either apoptotic or necrotic based on morphological characteristics (Wyllie et al., Int. Rev. Cytol. 68: 251 (1980)). These two modes of cell death were also initially thought to occur via regulated (caspase-dependent) and non-regulated processes, respectively. More recent studies, however, demonstrate that the underlying cell death mechanisms resulting in these two phenotypes are much more complicated and under some circumstances interrelated. Furthermore, conditions that lead to necrosis can occur by either regulated caspase-independent or non-regulated processes.

One regulated caspase-independent cell death pathway with morphological features resembling necrosis, called necroptosis, has been described (Degterev et al., Nat. Chem. Biol. 1:112, 2005). This manner of cell death can be initiated with various stimuli (e.g., TNF-[alpha] and Fas ligand) and in an array of cell types (e.g., monocytes, fibroblasts, lymphocytes, macrophages, epithelial cells and neurons). Necroptosis may represent a significant contributor to and in some cases predominant mode of cellular demise under pathological conditions involving excessive cell stress, rapid energy loss and massive oxidative species generation, where the highly energy-dependent apoptosis process is not operative.

In WO2015/172203 (which claims priority to AU2014903569 and AU2014901804), we reported that particular compounds described in US2005/0085637 have been found to be suitable for inhibiting necroptosis.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY OF THE INVENTION

As discussed above, certain compounds described in US2005/0085637 and WO2015/172203 have been found to be suitable for inhibiting necroptosis. Surprisingly, the inventors of this invention have now discovered that specific variations to the structure of these compounds lead to unexpectedly improved biological activity. In some instances, the improvement is a greater potency in necroptosis inhibition. In other instances, the improvement is a decrease in off-target activity, suggesting a likely decrease in toxicity. With some compounds, both an increase in potency in necroptosis inhibition and a decrease in off-target activity is observed.

In one aspect, the present invention provides a compound of Formula (I):

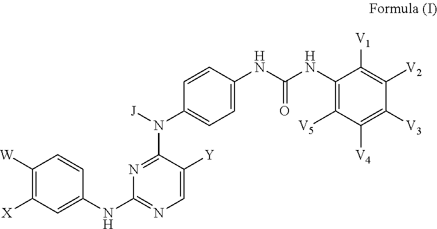

or a salt, solvate, or prodrug thereof

wherein

J is selected from hydrogen and methyl; and

Y is selected from hydrogen, methyl and halogen; and

W is selected from the group consisting of hydrogen, halogen, C₁-C₃ alkyl, C₁-C₃ haloalkyl, —OR¹ and (C₀-C₄ alkyl)C₃-C₇ heterocyclyl; and

X is selected from the group consisting of cyano, —OR¹, —(C₁-C₄ alkyl)NR³R⁴, C₃-C₇ cycloalkyl, (C₀-C₄ alkyl)C₃-C₇ heterocyclyl, aryl, heteroaryl, 4 to 7-membered lactam; and the group defined by -(A¹)ₘ-(A²)-(A³), wherein
A¹ is CH₂ and m is 0, 1, 2, or 3, or
A¹ is NR² and m is 0 or 1, or
A¹ is oxygen and m is 0 or 1, or
A¹ is CH₂NR² and m is 0 or 1;
A² is S(O)₂, S(O), or C(O); and
A³ is C₁-C₄ alkyl, C₁-C₄ alkoxy, C₁-C₄ hydroxyalkoxy, C₃-C₇ cycloalkyl, C₃-C₇ heterocyclyl, NR³R⁴, aryl, arylamino, aralkyl, aralkoxy, or heteroaryl;
R¹ is selected from the group consisting of hydrogen, C₁-C₄ alkyl, C₃-C₇ heterocyclyl, (C₀-C₄ alkyl)C₃-C₇ heterocyclyl and —NR³R⁴;
R², R³, and R⁴ are each independently selected from the group consisting of hydrogen, hydroxy, C₁-C₆ alkyl, C₁-C₆ alkoxy, aryloxy, aralkoxy, amino, C₁-C₆ alkylamino, arylamino, aralkylamino, C₁-C₄ alkyl, C₃-C₇ cycloalkyl, C₃-C₇ heterocyclyl, —S(O)₂R⁵, and —C(O)R⁵; and
R⁵ is selected from C₁-C₄ alkyl, or C₃-C₇ cycloalkyl.

V₁, V₂, V₃, V₄ and V₅ are each independently selected from hydrogen and a group defined by —(X₄)_{z}—(X₅), wherein
X₄ is CH₂ where z is 0, 1, 2, 3, or 4, and
X₅ is selected from the group consisting of hydrogen, C₁-C₆ alkyl, C₁-C₆ haloalkyl, C₃-C₇ cycloalkyl, C₃-C₇ heterocyclyl, aryl, heteroaryl, C₁-C₆ alkoxy, C₁-C₆ haloalkoxy, hydroxy, aryloxy, aralkoxy, halo, —CN, —NR′R′, N(H)C(O)R″, N(H)C(O)OR″, N(H)C(O)NR′R′, N(H)S(O)₂R″, OR″, OC(O)RR″, C(O)R″, SR″, S(O)R′″, S(O)₂ R′″, and S(O)₂NR′R′, wherein
R′ is selected from the group consisting of hydrogen, C₁-C₆ alkyl, C₃-C₇ cycloalkyl, C₃-C₇ heterocyclyl, —OR¹, —SR¹, —S(O)₂R¹, —S(O)R¹, and C(O)R¹;
R″ is selected from the group consisting of hydrogen, C₁-C₆ alkyl, C₃-C₇ cycloalkyl, C₃-C₇ heterocyclyl, —OR¹, —NR³R⁴, —S(O)₂R¹, —S(O)R¹ and C(O)R¹; and
R′″ is selected from the group consisting of hydrogen, C₁-C₆ alkyl, C₃-C₇ cycloalkyl, C₃-C₇ heterocyclyl, —OR¹ and —NR³R⁴;

provided that one or more of the following conditions is satisfied:
(i) Y is halo or methyl; and
(ii) X is selected from the group consisting of —CONR³R⁴, —(C₁-C₄ alkyl)-NR³R⁴, 4 to 7-membered lactam, heteroaryl, cyano, —OR¹ and

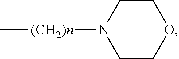

where D is O or NR⁶, wherein R⁶ is hydrogen or C₁-C₄ alkyl, and n is 1-4; and
(iii) V₁, V₃ and V₅ are hydrogen and V₂ and V₄ are each independently selected from the group consisting of halo, C₁-C₆ haloalkyl, C₁-C₆ alkyl and C₁-C₆ haloalkoxy.

In another aspect, there is provided a novel compound of Formula (I).

In one aspect, there is provided a composition comprising a compound according to Formula (I) or a salt, solvate, or prodrug thereof, and a pharmaceutically acceptable excipient.

In another aspect, there is provided a method for inhibiting necroptosis in a subject in need thereof, the method comprising administering a therapeutically effective amount of a compound or a salt, solvate, or prodrug thereof of Formula (I) to a subject.

In another aspect, there is provided a method for inhibiting necroptosis in a subject in need thereof, the method comprising administering a therapeutically effective amount of a composition containing a compound or a salt, solvate, or prodrug thereof of Formula (I) to a subject.

In another aspect, there is provided a method for inhibiting necroptosis in a subject in need thereof, the method comprising administering a therapeutically effective amount of a compound according to Formula (I) or a salt, solvate, or prodrug thereof, that binds to the ATP-binding site of the pseudokinase domain of Mixed Lineage Kinase Domain-like (MLKL) protein.

In another aspect, there is provided a method for inhibiting necroptosis in a subject in need thereof, the method comprising administering a therapeutically effective amount of a composition comprising a compound according to Formula (I) or a salt, solvate, or prodrug thereof, that binds to the ATP-binding site of the pseudokinase domain of Mixed Lineage Kinase Domain-like (MLKL) protein.

In another aspect, there is provided use of a compound of Formula (I) or a salt, solvate, or prodrug thereof, in the preparation of a medicament for the inhibition of necroptosis in a subject.

In another aspect, there is provided use of a composition comprising a compound of Formula (I) or a salt, solvate, or prodrug thereof, in the preparation of a medicament for the inhibition of necroptosis in a subject.

In another aspect, there is provided use of a compound according to Formula (I) or a salt, solvate, or prodrug thereof, for inhibiting necroptosis.

In another aspect, there is provided use of a composition comprising a compound according to Formula (I) or a salt, solvate, or prodrug thereof, for inhibiting necroptosis.

In yet another aspect, there is provided a compound according to Formula (I) or a salt, solvate, or prodrug thereof, for use in inhibiting necroptosis.

In another aspect, there is provided a composition comprising a compound according to Formula (I) or a salt, solvate, or prodrug thereof, for use in inhibiting necroptosis.

In yet another aspect, there is provided a compound according to Formula (I) or a salt, solvate, or prodrug thereof, when used for inhibiting necroptosis.

In yet another aspect, there is provided a composition comprising a compound according to Formula (I) or a salt, solvate, or prodrug thereof, when used for inhibiting necroptosis.

Any embodiment herein shall be taken to apply mutatis mutandis to any other embodiment unless specifically stated otherwise.

The present disclosure is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### DETAILED DESCRIPTION OF THE INVENTION

In one aspect, the present invention provides a compound of Formula (I):

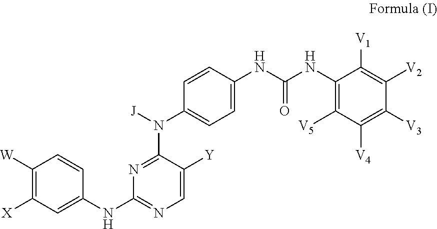

or a salt, solvate, or prodrug thereof

wherein

J is selected from hydrogen and methyl; and

Y is selected from hydrogen, methyl and halogen; and

W is selected from the group consisting of hydrogen, halogen, C₁-C₃ alkyl, C₁-C₃ haloalkyl, —OR¹ and (C₀-C₄ alkyl)C₃-C₇ heterocyclyl; and

X is selected from the group consisting of cyano, —OR¹, —(C₁-C₄ alkyl)NR³R⁴, C₃-C₇ cycloalkyl, (C₀-C₄ alkyl)C₃-C₇ heterocyclyl, aryl, heteroaryl, 4 to 7-membered lactam; and the group defined by -(A¹)ₘ-(A²)-(A³), wherein
A¹ is CH₂ and m is 0, 1, 2, or 3, or
A¹ is NR² and m is 0 or 1, or
A¹ is oxygen and m is 0 or 1, or
A¹ is CH₂NR² and m is 0 or 1;
A² is S(O)₂, S(O), or C(O); and
A³ is C₁-C₄ alkyl, C₁-C₄ alkoxy, C₁-C₄ hydroxyalkoxy, C₃-C₇ cycloalkyl, C₃-C₇ heterocyclyl, NR³R⁴, aryl, arylamino, aralkyl, aralkoxy, or heteroaryl;
R¹ is selected from the group consisting of hydrogen, C₁-C₄ alkyl, C₃-C₇ heterocyclyl, (C₀-C₄ alkyl)C₃-C₇ heterocyclyl and —NR³R⁴;
R², R³, and R⁴ are each independently selected from the group consisting of hydrogen, hydroxy, C₁-C₆ alkyl, C₁-C₆ alkoxy, aryloxy, aralkoxy, amino, C₁-C₆ alkylamino, arylamino, aralkylamino, C₁-C₄ alkyl, C₃-C₇ cycloalkyl, C₃-C₇ heterocyclyl, —S(O)₂R⁵, and —C(O)R⁵; and
R⁵ is selected from C₁-C₄ alkyl, or C₃-C₇ cycloalkyl.

V₁, V₂, V₃, V₄ and V₅ are each independently selected from hydrogen and a group defined by —(X₄)_{z}—(X₅), wherein
X₄ is CH₂ where z is 0, 1, 2, 3, or 4, and
X₅ is selected from the group consisting of hydrogen, C₁-C₆ alkyl, C₁-C₆ haloalkyl, C₃-C₇ cycloalkyl, C₃-C₇ heterocyclyl, aryl, heteroaryl, C₁-C₆ alkoxy, C₁-C₆ haloalkoxy, hydroxy, aryloxy, aralkoxy, halo, —CN, —NR′R′, N(H)C(O)R″, N(H)C(O)OR″, N(H)C(O)NR′R′, N(H)S(O)₂R″, OR″, OC(O)RR″, C(O)R″, SR″, S(O)R′″, S(O)₂ R′″, and S(O)₂NR′R′, wherein
R′ is selected from the group consisting of hydrogen, C₁-C₆ alkyl, C₃-C₇ cycloalkyl, C₃-C₇ heterocyclyl, —OR¹, —SR¹, —S(O)₂R¹, —S(O)R¹, and C(O)R¹;
R″ is selected from the group consisting of hydrogen, C₁-C₆ alkyl, C₃-C₇ cycloalkyl, C₃-C₇ heterocyclyl, —OR¹, —NR³R⁴, —S(O)₂R¹, —S(O)R¹ and C(O)R¹; and
R′″ is selected from the group consisting of hydrogen, C₁-C₆ alkyl, C₃-C₇ cycloalkyl, C₃-C₇ heterocyclyl, —OR¹ and —NR³R⁴;

provided that one or more of the following conditions is satisfied:
(i) Y is halo or methyl; and
(ii) X is selected from the group consisting of —CONR³R⁴, —(C₁-C₄ alkyl)-NR³R⁴, 4 to 7-membered lactam, heteroaryl, cyano, —OR¹ and

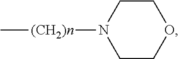

where D is O or NR⁶, wherein R⁶ is hydrogen or C₁-C₄ alkyl, and n is 1-4; and
(iii) V₁, V₃ and V₅ are hydrogen and V₂ and V₄ are each independently selected from the group consisting of halo, C₁-C₆ haloalkyl, C₁-C₆ alkyl and C₁-C₆ haloalkoxy.

In a preferred embodiment, J is methyl.

In another preferred embodiment, W is methyl.

Substitution at X

In one embodiment, an improvement in the biological activity of the compounds was provided by varying the group at position X in Formula (I). In the prior art, although some groups, including amides, were investigated, a sulphonamide group was the functional group of choice at this position. The inventors have found that selection of specific amide functionalities at this position leads to a surprising decrease in off-target activity. This was evident for amides including cyclic amides (i.e. lactams), and other specific groups including amines, substituted and non-substituted heteroaryl groups such as tetrazole, —CN, —OR¹, alkylmorpholino groups and alkylpiperazine groups. For example:

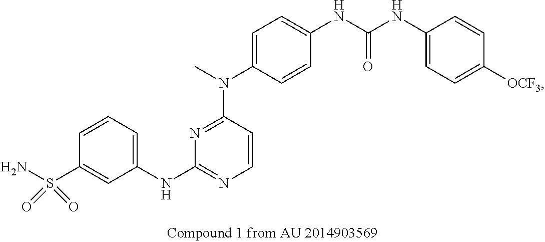 [SMILES: CN(c1ccc(NC(=O)Nc2ccc(OC(F)(F)F)cc2)cc1)c1ccnc(Nc2cccc(S(N)(=O)=O)c2)n1]

| | |
|---|---|
| 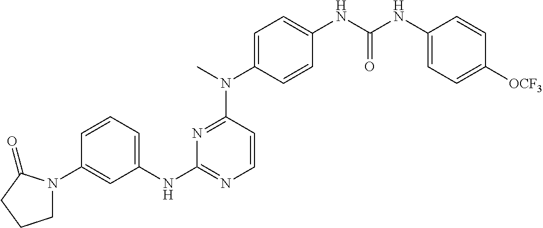 [SMILES: CN(c1ccc(NC(=O)Nc2ccc(OC(F)(F)F)cc2)cc1)c1ccnc(Nc2cccc(N3CCCC3=O)c2)n1] | (8) |

| | IC50 (nM) | IC50 (nM) |
|---|---|---|
| Compound | Necroptosis inhibition | Off-Target Effect |
| Compound 1 from AU 2014903569 | 67.9 | 3,566 |
| (8) | 75.4 | >10,000 |

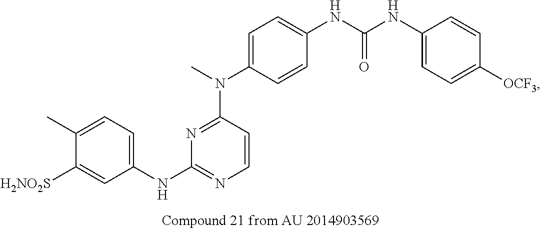 [SMILES: Cc1ccc(Nc2nccc(N(C)c3ccc(NC(=O)Nc4ccc(OC(F)(F)F)cc4)cc3)n2)cc1S(N)(=O)=O]

| | |
|---|---|
| 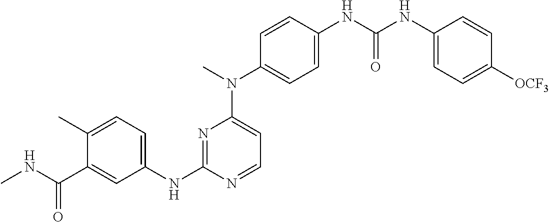 [SMILES: CNC(=O)c1cc(Nc2nccc(N(C)c3ccc(NC(=O)Nc4ccc(OC(F)(F)F)cc4)cc3)n2)ccc1C] | (3) |

| | IC50 (nM) | IC50 (nM) |
|---|---|---|
| Compound | Necroptosis inhibition | Off-Target Effect |
| Compound 21 from AU 2014903569 | 72.7 | 3,600 |
| (3) | 93.1 | >10,000 |

In certain embodiments, therefore, the present invention provides compounds of Formula (I) wherein X is —CONR³R⁴. In one embodiment, R³ and R⁴ are both hydrogen or both methyl. In another embodiment, R³ is hydrogen and R⁴ is methyl. In yet another embodiment, R³ is hydrogen and R⁴ is —CH₂CH₂OCH₃.

In other embodiments, the present invention provides compounds of Formula (I) wherein X is —CH₂N(CH₃)₂.

In other embodiments, the present invention provides compounds of Formula (I) wherein X is a 4 to 7-membered lactam, which may be substituted. Preferably it is a 5 to 7-membered lactam, and more preferably a 5-membered lactam. In another preferred embodiment, the lactam is part of a Spiro compound.

In yet another embodiment, X is an oxo-substituted heterocyclyl group. For example, in one embodiment, X is

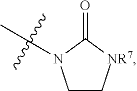

wherein R⁷ is hydrogen or C₁-C₄ alkyl.

In another embodiment, the present invention provides compounds of Formula (I) wherein X is heteroaryl, preferably methyl-substituted tetrazole.

In another embodiment, the present invention provides compounds of Formula (I) wherein X is —CH₂N(CH₃)₂.

In yet another embodiment, the present inevention provides compounds of Formula (I) wherein X is —CH₂-morpholine or —CH₂-piperazine, optionally substituted with C₁-C₆ alkyl. For example;

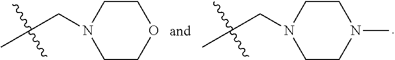

Substitution at Y

In another aspect, the addition of a halo- or methyl group on the central ring at position Y of Formula (I) was also found to improve the biological profile of these compounds, typically through greater potency observed for necroptosis inhibition. This compares favourably to other substituents at this position, such as a trifluoromethyl group, which surprisingly lost all efficacy for necroptosis inhibition. An example demonstrating this comparison is shown below.

| | |
|---|---|
| 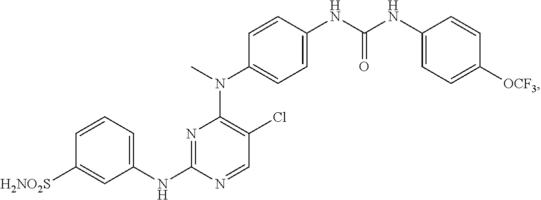 [SMILES: CN(c1ccc(NC(=O)Nc2ccc(OC(F)(F)F)cc2)cc1)c1nc(Nc2cccc(S(N)(=O)=O)c2)ncc1Cl] | (2) |
| 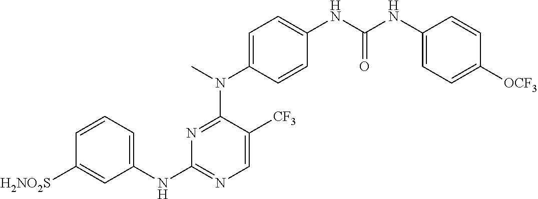 [SMILES: CN(c1ccc(NC(=O)Nc2ccc(OC(F)(F)F)cc2)cc1)c1nc(Nc2cccc(S(N)(=O)=O)c2)ncc1C(F)(F)F] | (6a) |

| | IC50 (nM) | IC50 (nM) |
|---|---|---|
| Compound | Necroptosis inhibition | Off-target Effect |
| Compound 1 from AU 2014903569 | 67.9 | 3,566 |
| (2) | 29.5 | 2110 |
| (6a) | >10,000 | 5,068 |

In certain embodiments, therefore, the present invention provides compounds of Formula (I) wherein Y is halo or methyl. In preferred embodiments, Y is chloro or fluoro.

Substitution at V

In another aspect, the selection of certain substituents on the phenyl ring at position V shown in Formula (I) above resulted in an increase in necroptosis inhibition and/or a decrease in off-target activity. This was particularly well observed when two meta-substituents were introduced on the ring, as seen below.

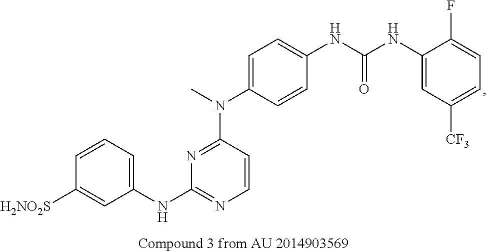 [SMILES: CN(c1ccc(NC(=O)Nc2cc(C(F)(F)F)ccc2F)cc1)c1ccnc(Nc2cccc(S(N)(=O)=O)c2)n1]

| | |
|---|---|
| 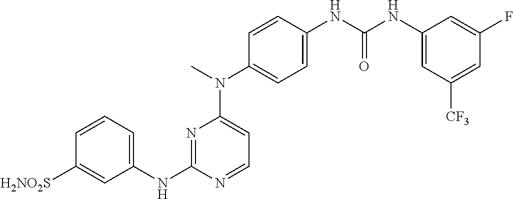 [SMILES: CN(c1ccc(NC(=O)Nc2cc(F)cc(C(F)(F)F)c2)cc1)c1ccnc(Nc2cccc(S(N)(=O)=O)c2)n1] | (10) |

| | IC50 (nM) | IC50 (nM) |
|---|---|---|
| Compound | Necroptosis inhibition | Off-target Effect |
| Compound 3 from AU 2014903569 | 15.3 | 791 |
| (10) | <1 | 506 |

In certain embodiments, therefore, the present invention provides compounds of Formula (I) wherein V₁, V₃ and V₅ are hydrogen and V₂ and V₄ are each independently selected from halo, C₁-C₆ haloalkyl, C₁-C₆ alkyl and C₁-C₆ haloalkoxy. Preferably, V₂ and V₄ are each independently selected from —F, —Cl, —OCF₃ and —CF₃.

Compounds with Several Different Substitutions:

In another aspect, the inventors reviewed variations to the core structure, attempting to take the favourable qualities of the modifications discussed above to prepare compounds displaying good necroptosis inhibition and low off-target activity.

For example, by adding a halo group at central carbon position Y to obtain high potency for necroptosis inhibition and adding an amide substituent at X, together with a methyl group at W to achieve low off-target activity, an excellent biological profile was obtained, as shown below with (12).

| | |
|---|---|
| 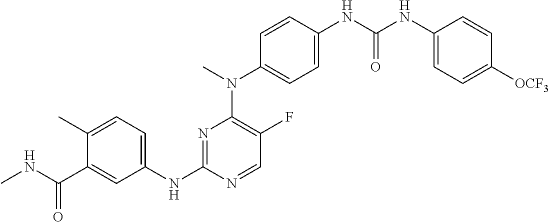 [SMILES: CNC(=O)c1cc(Nc2ncc(F)c(N(C)c3ccc(NC(=O)Nc4ccc(OC(F)(F)F)cc4)cc3)n2)ccc1C] | (12) |

| | IC50 (nM) | IC50 (nM) |
|---|---|---|
| Compound | Necroptosis inhibition | Off-target Effect |
| Compound 1 from AU 2014903569 | 67.9 | 3,566 |
| (12) | 10.1 | >10,000 |

In certain embodiments, therefore, the present invention provides compounds of Formula (I) wherein a combination of two or more of the preferred embodiments described herein are provided.

In a further aspect of the invention, there is provided novel compounds of Formula (I).

In yet another aspect, there is provided a composition comprising a compound according to Formula (I) or a salt, solvate, or prodrug thereof, and a pharmaceutically acceptable excipient.

As used herein the term “alkyl” refers to a straight or branched chain hydrocarbon radical having from one to twelve carbon atoms, or any range between, i.e. it contains 1, 2, 3, 4, 5, 6, 7, 8, 9, 10, 11 or 12 carbon atoms. The alkyl group is optionally substituted with substituents, multiple degrees of substitution being allowed. Examples of “alkyl” as used herein include, but are not limited to, methyl, ethyl, n-propyl, isopropyl, n-butyl, isobutyl, t-butyl, n-pentyl, isopentyl, and the like.

As used herein, the terms “C₁-C₃ alkyl”, “C₁-C₄ alkyl” and “C₁-C₆ alkyl” refer to an alkyl group, as defined above, containing at least 1, and at most 3, 4 or 6 carbon atoms respectively, or any range in between (e.g. alkyl groups containing 2-5 carbon atoms are also within the range of C₁-C₆). Where the term “C₀-C₂ alkyl” is used, there may be no alkyl group, or an alkyl group containing 1 or 2 carbon atoms.

As an example of substituted alkyls, the term —(C₁-C₄ alkyON(C₁-C₄ alkyl)₂ includes —CH₂N(CH₃)₂, —(CH₂)₂N(CH₃)₂, —CH₂N(CH₂CH₃)₂, —CH₂N(iPr)(CH₃), and the like.

As used herein, the term “halogen” refers to fluorine (F), chlorine (Cl), bromine (Br), or iodine (I) and the term “halo” refers to the halogen radicals fluoro (—F), chloro (—Cl), bromo (—Br), and iodo (—I). Preferably, ‘halo’ is fluoro or chloro.

As used herein, the term “cycloalkyl” refers to a non-aromatic cyclic hydrocarbon ring. In a like manner the term “C₃-C₇ cycloalkyl” refers to a non-aromatic cyclic hydrocarbon ring having from three to seven carbon atoms, or any range in between. For example, the C₃-C₇ cycloalkyl group would also include cycloalkyl groups containind 4 to 6 carbon atoms. The alkyl group is as defined above, and may be substituted. Exemplary “C₃-C₇ cycloalkyl” groups useful in the present invention include, but are not limited to, cyclopropyl, cyclobutyl, cyclopentyl, cyclohexyl and cycloheptyl.

As used herein, the terms “heterocyclic” or “heterocyclyl” refer to a nonaromatic heterocyclic ring, being saturated or having one or more degrees of unsaturation, containing one or more heteroatom substitution selected from S, S(O), S(O)₂, O, or N. The heterocyclyl group may be attached through any atom of its structure, including a heteroatom. The term “C₃-C₇ heterocyclyl” refers to a non-aromatic cyclic hydrocarbon ring having from three to seven carbon atoms containing one or more heteroatom substitutions as referred to herein. The heterocyclic moiety may be substituted, multiple degrees of substitution being allowed. The term “C₃-C₇ heterocyclyl” also includes heterocyclyl groups containing C₄-C₅, C₅-C₇, C₆-C₇, C₄-C₇, C₄-C₆ and C₅-C₆ carbon atoms. Preferably, the heterocyclic ring contains four to six carbon atoms and one or two heteroatoms. More preferably, the heterocyclic ring contains five carbon atoms and one heteroatom, or four carbon atoms and two heteroatom substitutions, or five carbon atoms and one heteroatom. Such a ring may be optionally fused to one or more other “heterocyclic” ring(s) or cycloalkyl ring(s). Examples of “heterocyclic” moieties include, but are not limited to, tetrahydrofuran, pyran, oxetane, 1,4-dioxane, 1,3-dioxane, piperidine, piperazine, N-methylpiperazinyl, 2,4-piperazinedione, pyrrolidine, imidazolidine, pyrazolidine, morpholine, thiomorpholine, tetrahydrothiopyran, tetrahydrothiophene, and the like.

As an example of substituted heterocyclic groups, the term “(C₀-C₄ alkyl)C₃-C₇ heterocyclyl” includes heterocyclyl groups containing either no alkyl group as a linker between the compound and the heterocycle, or an alkyl group containing 1, 2, 3 or 4 carbon atoms as a linker between the compound and the heterocycle (eg. heterocycle, —CH₂-heterocycle or —CH₂CH₂-heterocycle). The alkyl linker can bind to any atom of the heterocyclyl group, including a heteroatom. Any of these heterocycles may be further substituted.

An example of a substituted hereocyclic group is

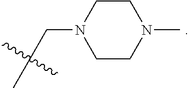 [SMILES: *CN1CCN(C)CC1]

Substituted cycloalkyl and heterocyclyl groups may be substituted with any suitable substituent as described below. They may be substituted at any of the carbons on the ring with another cycloalkyl or heterocyclic moiety to form a spiro compound.

As used herein, the term “aryl” refers to an optionally substituted benzene ring or to an optionally substituted benzene ring system fused to one or more optionally substituted benzene rings to form, for example, anthracene, phenanthrene, or napthalene ring systems. Examples of “aryl” groups include, but are not limited to, phenyl, 2-naphthyl, 1-naphthyl, biphenyl, as well as substituted derivatives thereof. Preferred aryl groups include arylamino, aralkyl, aralkoxy, heteroaryl groups.

As used herein, the term “heteroaryl” refers to a monocyclic five, six or seven membered aromatic ring, or to a fused bicyclic or tricyclic aromatic ring system comprising at least one monocyclic five, six or seven membered aromatic ring. These heteroaryl rings contain one or more nitrogen, sulfur, and/or oxygen heteroatoms, where N-oxides and sulfur oxides and dioxides are permissible heteroatom substitutions and may be optionally substituted with up to three members. Examples of “heteroaryl” groups used herein include furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridyl, pyridazyl, pyrazinyl, pyrimidyl, quinolinyl, isoquinolinyl, benzofuranyl, benzothiophenyl, indolyl, indazolyl, benzimidazolyl, and substituted versions thereof.

As used herein, the term “4 to 7-membered lactam” refers to lactam rings made up of 4 to 7 members, including the nitrogen atom. This can include substituted lactams. Examples of non-substituted 4 to 7-membered lactams are illustrated below. Preferably, the 4 to 7-membered lactam is a 5-membered lactam.

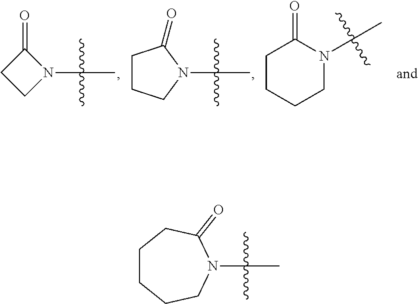 [SMILES: *N1CCC1=O ; *N1CCCC1=O ; *N1CCCCC1=O ; *N1CCCCCC1=O]

Substituted lactams may be substituted with any suitable substituent as described below. They may also be substituted at any of the carbons in the lactam ring with a cyclic or heterocyclic moiety to form a Spiro substituent Examples of substituted 4 to 6-membered lactams forming Spiro substituents include, but are not limited to:

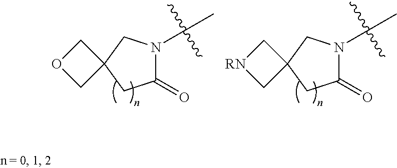

A “substituent” as used herein, refers to a molecular moiety that is covalently bonded to an atom within a molecule of interest. For example, a “ring substituent” may be a moiety such as a halogen, alkyl group, or other substituent described herein that is covalently bonded to an atom, preferably a carbon or nitrogen atom, that is a ring member. The term “substituted,” as used herein, means that any one or more hydrogens on the designated atom is replaced with a selection from the indicated substituents, provided that the designated atom's normal valence is not exceeded, and that the substitution results in a stable compound, i.e., a compound that can be isolated, characterized and tested for biological activity.

The terms “optionally substituted” or “may be substituted” and the like, as used throughout the specification, denotes that the group may or may not be further substituted or fused (so as to form a polycyclic system), with one or more non-hydrogen substituent groups. Suitable chemically viable subtituents for a particular functional group will be apparent to those skilled in the art.

Examples of substituents include but are not limited to:

C₁-C₆ alkyl, C₁-C₆ haloalkyl, C₁-C₆ haloalkoxy, C₁-C₆ hydroxyalkyl, C₁-C₆ hydroxyalkoxy, C₃-C₇ heterocyclyl, C₃-C₇ cycloalkyl, C₁-C₆ alkoxy, C₁-C₆ alkylsulfanyl, C₁-C₆ alkylsulfenyl, C₁-C₆ alkylsulfonyl, C₁-C₆ alkylsulfonylamino, arylsulfonoamino, alkylcarboxy, alkylcarboxyamide, oxo, hydroxy, mercapto, amino, acyl, carboxy, carbamoyl, aryl, aryloxy, heteroaryl, aminosulfonyl, aroyl, aroylamino, heteroaroyl, acyloxy, aroyloxy, heteroaroyloxy, alkoxycarbonyl, nitro, cyano, halogen, ureido or C₁-C₆ perfluoroalkyl. In one embodiment, cyclic or heterocyclic substituents may form a Spiro substituent with a carbon in the moiety from which the cyclic or heterocyclic group is substituted.

Any of these groups may be further substituted by any of the above-mentioned groups, where appropriate. For example, alkylamino, or dialkylamino, C₁-C₆ alkoxy, etc.

In one aspect of the present invention, there is provided a method for inhibiting necroptosis in a subject in need thereof, the method comprising administering a therapeutically effective amount of a compound or a salt, solvate, or prod rug thereof of Formula (I) to a subject.

In another aspect, there is provided a method for inhibiting necroptosis in a subject in need thereof, the method comprising administering a therapeutically effective amount of a composition containing a compound or a salt, solvate, or prodrug thereof of Formula (I) to a subject.

In another aspect of the present disclosure, there is provided a method for inhibiting necroptosis in a subject in need thereof, the method comprising administering a therapeutically effective amount of a compound according to Formula (I) or a salt, solvate, or prodrug thereof, that binds to the ATP-binding site of the pseudokinase domain of Mixed Lineage Kinase Domain-like (MLKL) protein.

In another aspect of the present disclosure, there is provided a method for inhibiting necroptosis in a subject in need thereof, the method comprising administering a therapeutically effective amount of a composition comprising a compound according to Formula (I) or a salt, solvate, or prodrug thereof, that binds to the ATP-binding site of the pseudokinase domain of Mixed Lineage Kinase Domain-like (MLKL) protein.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

In one embodiment of the present disclosure, administration of a compound according to Formula (I) inhibits a conformational change of MLKL. In another embodiment, the conformational change of MLKL involves release of the four-helix bundle (4HB) domain of MLKL. In another embodiment, administration of the compound inhibits oligomerisation of MLKL. In yet another embodiment, administration of the compound inhibits translocation of MLKL to the cell membrane. In a further embodiment, administration of the compound inhibits a conformational change of MLKL, inhibits oligomerisation of MLKL and inhibits translocation of MLKL to the cell membrane.

It is envisaged that some compounds of the present disclosure can bind to MLKL in various species and inhibit necroptosis.

As used herein, the term “pseudokinase domain” as understood by a person skilled in the art, means a protein containing a catalytically-inactive or catalytically-defective kinase domain. “Pseudokinase domains” are often referred to as “protein kinase-like domains” as these domains lack conserved residues known to catalyse phosphoryl transfer. It would be understood by a person skilled in the art that although pseudokinase domains are predicted to function principally as catalysis independent protein-interaction modules, several pseudokinase domains have been attributed unexpected catalytic functions. Accordingly, in the present disclosure the term “pseudokinase domain” includes “pseudokinase domains” which lack kinase activity and “pseudokinase domains” which possess weak kinase activity.

As used herein, the term “ATP-binding site” as understood by a person skilled in the art, means a specific sequence of protein subunits that promotes the attachment of ATP to a target protein. An ATP binding site is a protein micro-environment where ATP is captured and hydrolyzed to ADP, thereby releasing energy that is utilized by the protein to work by changing the protein shape and/or making the enzyme catalytically active. In pseudokinase domains, the “ATP-binding site” is often referred to as the “pseudoactive site”. The term “ATP-binding site” may also be referred to as a “nucleotide-binding site” as binding at this site includes the binding of nucleotides other than ATP. It would be understood by a person skilled in the art that the term “nucleotide” includes any nucleotide. Exemplary nucleotides include, but are not limited to, AMP, ADP, ATP, AMPPNP, GTP, CTP and UTP.

As described herein, inhibition of necroptosis includes both complete and partial inhibition of necroptosis. In one embodiment, inhibition of necroptosis is complete inhibition. In another embodiment, inhibition of necroptosis is partial inhibition.

The binding of a compound to the ATP-binding site of the pseudokinase domain of MLKL may be determined by any method considered to be suitable by a person skilled in the art for such a use. In one embodiment of the present disclosure, the binding of a compound to the ATP-binding site of the pseudokinase domain of MLKL is determined by one or more assays selected from the group comprising, but not limited to, thermal shift assay, surface plasmon resonance (SPR), and saturation transfer difference NMR (STD-NMR). In another embodiment, the binding of a compound to the ATP-binding site of the pseudokinase domain of MLKL is determined by thermal shift assay. In yet another embodiment, the binding of a compound to the ATP-binding site of the pseudokinase domain of MLKL is determined by SPR. In yet another embodiment, the binding of a compound to the ATP-binding site of the pseudokinase domain of MLKL is determined by STD-NMR. In a further embodiment, the binding of a compound to the ATP-binding site of the pseudokinase domain of MLKL is determined by thermal shift assay and one or more additional assays. In yet a further embodiment, the additional assays are selected from the group comprising, but not limited to, SPR and STD-NMR.

A thermal shift assay, also called Differential Scanning Fluorimetry (DSF) is a thermal-denaturation assay that measures the thermal stability of a target protein and a subsequent increase in protein melting temperature upon binding of a ligand to the protein. The binding of low molecular weight ligands can increase the thermal stability of a protein and the thermal stability change is measured by performing a thermal denaturation curve in the presence of a fluorescent dye. The fluorescent dye used is typically a non-specific dye (such as SYPRO Orange) and binds nonspecifically to hydrophobic surfaces, and water strongly quenches the fluorescence of the fluorescent dye. When the protein unfolds, the exposed hydrophobic surfaces bind the dye, resulting in an increase in fluorescence. The stability curve and its midpoint value for the protein unfolding transition (melting temperature, Tₘ) are obtained by gradually increasing the temperature to unfold the protein and measuring the fluorescence at each point. Curves are measured for protein only and protein plus ligand, and the ΔTₘ is calculated. A positive ΔTₘ value indicates that the ligand stabilizes the protein from denaturation, and therefore binds the protein. A fluorescence-based thermal shift assay can be performed on instruments that combine sample temperature control and dye fluorescence detection, such as readily available real-time polymerase chain reaction (RT-PCR) machines.

The surface plasmon resonance (SPR) technique is a well-established method for the measurement of molecules binding to surfaces and the quantification of binding constants between surface-immobilized proteins and an analyte such as other proteins, peptides, nucleic acids, lipids or small molecules in solution without the use of labels. The SPR effect relies on changes in the refractive index of solutions adjacent to the immobilised surface and is extremely sensitive. Binding responses are measured in resonance units (RU) and are proportional to the molecular mass on the sensor chip surface and, consequently, to the number of molecules on the surface. The affinity of the interaction can be calculated from the ratio of the rate constants (K_{d}=k_{diss}/kₐₛₛ) or by a linear or nonlinear fitting of the response at equilibrium at varying concentrations of analyte.

Saturation transfer difference NMR (STD-NMR) allows for the detection of transient binding of small molecule ligands to macromolecular receptors such as proteins. In STD-NMR, magnetization transferred from the receptor to its bound ligand is measured by directly observing NMR signals from the ligand itself. Low-power irradiation is applied to a (1)H NMR spectral region containing protein signals but no ligand signals. This irradiation spreads quickly throughout the membrane protein by the process of spin diffusion and saturates all protein (1)H NMR signals. (1)H NMR signals from a ligand bound transiently to the membrane protein become saturated and, upon dissociation, serve to decrease the intensity of the (1)H NMR signals measured from the pool of free ligand. The experiment is repeated with the irradiation pulse placed outside the spectral region of protein and ligand, a condition that does not lead to saturation transfer to the ligand. The two resulting spectra are subtracted to yield the difference spectrum. The resulting difference spectrum yields only those resonances that have experienced saturation, namely those of the receptor and those of the compound that binds to the receptor. STD-NMR can therefore be used to determine the binding epitope of the compound. Competition STD-NMR methods combine STD-NMR with competition binding experiments to allow the detection of high-affinity ligands that undergo slow chemical exchange on the NMR time-scale. With this technique, the presence of a competing high-affinity ligand in the compound mixture can be detected by the disappearance or reduction of the STD signals of a low-affinity indicator ligand. This method can therefore be used to derive the binding affinity (K_{d}) of compounds based on the reduction of the signal intensity of the STD indicator.

A compound that binds to the ATP-binding site of the pseudokinase domain of the MLKL protein, as described herein, may be any compound according to Formula (I) or a salt, solvate, or prodrug thereof which performs the described function and thereby effects the inhibition of necroptosis.

Binding of the compound to the ATP-binding site of the pseudokinase domain of MLKL may inhibit phosphorylation of MLKL by an effector kinase or binding of the compound to the ATP-binding site of the pseudokinase domain of MLKL may not inhibit phosphorylation of MLKL by an effector kinase. The present disclosure demonstrates that compounds that bind to the ATP-binding site of the pseudokinase domain of the MLKL protein, as described herein, can inhibit necroptosis without inhibiting phosphorylation of MLKL by an effector kinase. In one embodiment, binding of the compound to the ATP-binding site of the pseudokinase domain of MLKL does not inhibit phosphorylation of MLKL by an effector kinase. In another embodiment, binding of the compound to the ATP-binding site of the pseudokinase domain of MLKL inhibits phosphorylation of MLKL by an effector kinase.

In another aspect, there is provided use of a compound of Formula (I) or a salt, solvate, or prodrug thereof in the preparation of a medicament for the inhibition of necroptosis in a subject.

In another aspect, there is provided use of a composition comprising a compound of Formula (I) or a salt, solvate, or prodrug thereof in the preparation of a medicament for the inhibition of necroptosis in a subject.

In another aspect, there is provided use of a compound of Formula (I) or a salt, solvate, or prodrug thereof for inhibiting necroptosis.

In another aspect, there is provided use of a composition comprising a compound of Formula (I) or a salt, solvate, or prodrug thereof for inhibiting necroptosis.

In yet another aspect, there is provided a compound according to Formula (I) or a salt, solvate, or prodrug thereof for use in inhibiting necroptosis.

In yet another aspect, there is provided a composition comprising a compound according to Formula (I) or a salt, solvate, or prodrug thereof for use in inhibiting necroptosis.

In yet another aspect, there is provided a compound according to Formula (I) or a salt, solvate, or prodrug thereof when used for inhibiting necroptosis.

In yet another aspect, there is provided a composition comprising a compound according to Formula (I) or a salt, solvate, or prodrug thereof when used for inhibiting necroptosis.

The salts of the compounds of Formula (I) are preferably pharmaceutically acceptable, but it will be appreciated that non-pharmaceutically acceptable salts also fall within the scope of the present disclosure, since these are useful as intermediates in the preparation of pharmaceutically acceptable salts.

The term “pharmaceutically acceptable” may be used to describe any pharmaceutically acceptable salt, hydrate or prodrug, or any other compound which upon administration to a subject, is capable of providing (directly or indirectly) a compound of Formula (I) or an active metabolite or residue thereof.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine. General information on types of pharmaceutically acceptable salts and their formation is known to those skilled in the art and is as described in general texts such as “Handbook of Pharmaceutical salts” P. H. Stahl, C. G. Wermuth, 1st edition, 2002, Wiley-VCH.

In the case of compounds that are solids, it will be understood by those skilled in the art that the inventive compounds, agents and salts may exist in different crystalline or polymorphic forms, all of which are intended to be within the scope of the present invention and specified formulae.

The term “polymorph” includes any crystalline form of compounds of Formula (I), such as anhydrous forms, hydrous forms, solvate forms and mixed solvate forms.

Formula (I) is intended to cover, where applicable, solvated as well as unsolvated forms of the compounds. Thus, formula (I) includes compounds having the indicated structure, including the hydrated or solvated form, as well as the non-hydrated and non-solvated forms.

As used herein, the term “solvate” refers to a complex of variable stoichiometry formed by a solute (in this invention, a compound of formula (I) or a salt or prodrug thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

A “prodrug” is a compound that may not fully satisfy the structural requirements of the compounds provided herein, but is modified in vivo, following administration to a subject or patient, to produce a compound of formula (I) provided herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

Prodrugs include compounds wherein an amino acid residue, or a polypeptide chain of two or more (eg, two, three or four) amino acid residues which are covalently joined to free amino, and amido groups of compounds of Formula (I). The amino acid residues include the 20 naturally occurring amino acids commonly designated by three letter symbols and also include, 4-hydroxyproline, hydroxylysine, demosine, isodemosine, 3-methylhistidine, norvlin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of Formula (I) through the carbonyl carbon prodrug sidechain.

The compounds of Formula (I) and prodrugs thereof may be covalent irreversible or covalent reversible inhibitors of the active site of a protein.

Pharmaceutical compositions may be formulated from compounds according to formula (I) for any appropriate route of administration including, for example, topical (for example, transdermal or ocular), oral, buccal, nasal, vaginal, rectal or parenteral administration. The term parenteral as used herein includes subcutaneous, intradermal, intravascular (for example, intravenous), intramuscular, spinal, intracranial, intrathecal, intraocular, periocular, intraorbital, intrasynovial and intraperitoneal injection, as well as any similar injection or infusion technique. In certain embodiments, compositions in a form suitable for oral use or parenteral use are preferred. Suitable oral forms include, for example, tablets, troches, lozenges, aqueous or oily suspensions, dispersible powders or granules, emulsions, hard or soft capsules, or syrups or elixirs. For intravenous, intramuscular, subcutaneous, or intraperitoneal administration, one or more compounds may be combined with a sterile aqueous solution which is preferably isotonic with the blood of the recipient. Such formulations may be prepared by dissolving solid active ingredient in water containing physiologically compatible substances such as sodium chloride or glycine, and having a buffered pH compatible with physiological conditions to produce an aqueous solution, and rendering said solution sterile. The formulations may be present in unit or multi-dose containers such as sealed ampoules or vials. Examples of components are described in Martindale—The Extra Pharmacopoeia (Pharmaceutical Press, London 1993) and Martin (ed.), Remington's Pharmaceutical Sciences.

In the context of this specification the term “administering” and variations of that term including “administer” and “administration”, includes contacting, applying, delivering or providing a compound or composition of the invention to an organism, or a surface by any appropriate means.

For the inhibition of necroptosis, the dose of the biologically active compound according to the invention may vary within wide limits and may be adjusted to individual requirements. Active compounds according to the present invention are generally administered in a therapeutically effective amount. Preferred doses range 5 from about 0.1 mg to about 140 mg per kilogram of body weight per day (e.g. about 0.5 mg to about 7 g per patient per day). The daily dose may be administered as a single dose or in a plurality of doses. The amount of active ingredient that may be combined with the carrier materials to produce a single dosage form will vary depending upon the subject treated and the particular mode of administration. Dosage unit forms will generally contain between about 1 mg to about 500 mg of an active ingredient.

It will be understood, however, that the specific dose level for any particular subject and will depend upon a variety of factors including the activity of the specific compound employed, the age, body weight, general health, sex, diet, time of administration, route of administration, and rate of excretion, drug combination (i.e. other drugs being used to treat the subject), and the severity of the particular disorder undergoing therapy. The dosage will generally be lower if the compounds are administered locally rather than systemically, and for prevention rather than for treatment. Such treatments may be administered as often as necessary and for the period of time judged necessary by the treating physician. A person skilled in the art will appreciate that the dosage regime or therapeutically effective amount of the compound of formula (I) to be administered may need to be optimized for each individual. The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight, may be appropriate. The daily dose can be administered in one to four doses per day.

It will also be appreciated that different dosages may be required for treating different disorders. An effective amount of an agent is that amount which causes a statistically significant decrease in necroptosis.

For in vitro analysis, the necroptosis inhibition may be determined by assays used to measure TSQ-induced necroptosis, as described in the biological tests defined herein.

The terms “therapeutically effective amount” or “effective amount” refer to an amount of the compound of formula (I) that results in an improvement or remediation of the symptoms of necroptosis and/or associated diseases or their symptoms.

The terms “treating”, “treatment” and “therapy” are used herein to refer to curative therapy, prophylactic therapy and preventative therapy. Thus, in the context of the present disclosure the term “treating” encompasses curing, ameliorating or tempering the severity of necroptosis and/or associated diseases or their symptoms.

“Preventing” or “prevention” means preventing the occurrence of the necroptosis or tempering the severity of the necroptosis if it develops subsequent to the administration of the compounds or pharmaceutical compositions of the present invention.

“Subject” includes any human or non-human animal. Thus, in addition to being useful for human treatment, the compounds of the present invention may also be useful for veterinary treatment of mammals, including companion animals and farm animals, such as, but not limited to dogs, cats, horses, cows, sheep, and pigs.

The term “inhibit” is used to describe any form of inhibition that results in prevention, reduction or otherwise amelioration of necroptosis, including complete and partial inhibition.

The compounds of the present invention may be administered along with a pharmaceutical carrier, diluent or excipient as described above.

The methods of the present disclosure can be used to prevent or treat the following diseases and/or conditions in a subject:
diseases of the bones, joints, connective tissue and of cartilage, such as osteoporosis, osteomyelitis, arthritises including for example osteoarthritis, rheumatoid arthritis and psoriatic arthritis, avascular necrosis, progressive fibrodysplasia ossificans, rickets, Cushing's syndrome;
muscular diseases such as muscular dystrophy, such as for example Duchenne's muscular dystrophy, myotonic dystrophies, myopathies and myasthenias;
diseases of the skin, such as dermatitis, eczema, psoriasis, aging or even alterations of scarring;
cardiovascular diseases such as cardiac and/or vascular ischemia, myocardium infarction, ischemic cardiopathy, chronic or acute congestive heart failure, cardiac dysrythmia, atrial fibrillation, ventricular fibrillation, paroxystic tachycardia, congestive heart failure, hypertrophic cardiopathy, anoxia, hypoxia, secondary effects due to therapies with anti-cancer agents;
circulatory diseases such as atherosclerosis, arterial scleroses and peripheral vascular diseases, cerebrovascular strokes, aneurisms;
haematological and vascular diseases such as: anemia, vascular amyloidosis, haemorrhages, drepanocytosis, red cell fragmentation syndrome, neutropenia, leukopenia, medullar aplasia, pantocytopenia, thrombocytopenia, haemophilia;
lung diseases including pneumonia, asthma; obstructive chronic diseases of the lungs such as for example chronic bronchitis and emphysema;
diseases of the gastro-intestinal tract, such as ulcers;
diseases of the liver such as for example hepatitis particularly hepatitis of viral origin or having as causative agent other infectious agents, auto-immune hepatitis, fulminating hepatitis, certain hereditary metabolic disorders, Wilson's disease, cirrhoses, non-alcoholic hepatic steatosis, diseases of the liver due to toxins and to drugs;
diseases of the pancreas such as for example acute or chronic pancreatitis;
metabolic diseases such as diabetes mellitus and insipid diabetes, thyroiditis;
diseases of the kidneys such as for example acute renal disorders or glomerulonephritis;
viral and bacterial infections such as septicemia;
severe intoxications by chemicals, toxins or drugs;
degenerative diseases associated with the Acquired Immune Deficiency Syndrome (AIDS);
disorders associated with aging such as the syndrome of accelerated aging;
inflammatory diseases such as Crohn's disease, rheumatoid polyarthritis;
auto-immune diseases such as erythematous lupus;
dental disorders such as those resulting in degradation of tissues such as for example periodontitis;
ophthalmic diseases or disorders including diabetic retinopathies, glaucoma, macular degenerations, retinal degeneration, retinitis pigmentosa, retinal holes or tears, retinal detachment, retinal ischemia, acute retinopathies associated with trauma, inflammatory degenerations, post-surgical complications, medicinal retinopathies, cataract;
disorders of the audition tracts, such as otosclerosis and deafness induced by antibiotics;
fibrosis
diseases associated with mitochondria (mitochondrial pathologies), such as Friedrich's ataxia, congenital muscular dystrophy with structural mitochondrial abnormality, certain myopathies (MELAS syndrome, MERFF syndrome, Pearson's syndrome), MIDD (mitochondrial diabetes and deafness) syndrome, Wolfram's syndrome, dystonia; and
cancer and metastasis including but not limited to cancers of the lung and bronchus, including non-small cell lung cancer (NSCLC), squamous lung cancer, brochioloalveolar carcinoma (BAC), adenocarcinoma of the lung, and small cell lung cancer (SCLC); prostate cancer, including androgen-dependent and androgen-independent prostate cancer; breast cancer, including metastatic breast cancer; pancreatic cancer; cancers of the colon and rectum; thyroid cancer; cancers of the liver and intrahepatic bile duct; hepatocellular cancer; gastric cancer; endometrial cancer; melanoma; cancers of the kidney, renal pelvis, urinary bladder, uterine corpus and uterine cervix; ovarian cancer, including progressive epithelial or primary peritoneal cancer; multiple myeloma; oesophageal cancer, including squamous cell carcinoma and adenocarcinoma of the oesophagus; acute myelogenous leukemia (AML); chronic myelogenous leukemia (CML), including accelerated CML and CML blast phase (CML-BP); lymphocytic leukemia; myeloid leukemia; acute lymphoblastic leukemia (ALL); chronic lymphocytic leukemia (CLL); Hodgkin's disease (HD); non-Hodgkin's lymphoma (NHL), including follicular lymphoma and mantle cell lymphoma; B-cell lymphoma, including diffuse large B-cell lymphoma (DLBCL); T-cell lymphoma; multiple myeloma (MM); amyloidosis; Waldenstrom's macroglobulinemia; myelodysplastic syndromes (MDS), including refractory anemia (RA), refractory anemia with ringed siderblasts (RARS), (refractory anemia with excess blasts (RAEB), and RAEB in transformation (RAEB-T); and myeloproliferative syndromes; cancers of the brain, including glioma/glioblastoma, anaplastic oligodendroglioma, and adult anaplastic astrocytoma; neuroendocrine cancers, including metastatic neuroendocrine tumors; cancers of the head and neck, including , e.g., squamous cell carcinoma of the head and neck, and nasopharyngeal cancer; cancers of the oral cavity, pharynx and small intestine; bone cancer; soft tissue sarcoma; and villous colon adenoma.

The methods can also be used for protecting cells, tissues and/or transplanted organs, whether before, during (removal, transport and/or re-implantation) or after transplantation.

The methods and compounds described herein are described by the following illustrative and non-limiting examples.

### EXAMPLES

1.1 Materials and Methods

Compounds.

All temperatures referred to are in ° C.

The names of the following compounds have been obtained using ChemDraw Ultra 12.0.

Abbreviations

AcOH acetic acid

AlCl₃ aluminium chloride BINAP 2,2′-Bis(diphenylphosphino)-1,1′-binaphthyl (Boc)₂O di-tert-butyl dicarbonate

CDCl₃ deuterochloroform

CDI 1,1′-Carbonyldiimidazole

Cs₂CO₃ caesium carbonate

DMSO-d₆ deuterated dimethylsulfoxide

DCC dicyclohexylcarbodiimide

DCM dichloromethane

DIPEA diisopropylethylamine

DMF N,N-dimethylformamide

DMSO dimethylsulfoxide

TEA triethylamine

EtOAc ethylacetate

EtOH ethanol

hr hour(s)

HATU 1-[Bis(dimethylamino)methylene]-1H-1,2,3-triazolo[4,5-b] pyridinium 3-oxid hexafluorophosphate

HCl hydrochloric acid/hydrogen chloride

HPLC high performance liquid chromatography

K₂CO₃ potassium carbonate

LCMS liquid chromatography-mass spectrometry

M molar (concentration)

MeOH methanol

min minute(s)

M/Z mass/charge ratio (mass spectrometry)

Na₂CO₃ sodium carbonate

NaH sodium hydride

NaHCO₃ sodium bicarbonate

NaOH sodium hydroxide

Na₂SO₄ sodium sulphate

NH₄Cl ammonium chloride

NMP N-methyl-2-pyrrolidinone

NMR nuclear magnetic resonance

Pd/C palladium on activated charcoal

Pd₂(dba)₃ tris(dibenzylideneacetone)dipalladium(0)

Rt retention time

rt room temperature

SEM-Cl 2-(Trimethylsilyl)ethoxymethyl chloride

SOCl₂ thionyl chloride

TFA trifluoroacetic acid

THF tetrahydrofuran

TsOH tosyl chloride

Xantphos 4,5-Bis(diphenylphosphino)-9,9-dimethylxanthene

LCMS Methodology

Electrospray mass spectroscopy (MS) was carried out using the following method;

Method A (10 min method): Finnigan LCQ Advantage Max using reverse phase high performance liquid chromatorgraphy (HPLC) analysis (column: Gemini 3μ C18 20×4.0 mm 110A) Solvent A: Water 0.1% Formic Acid, Solvent B: Acetonitrile 0.1% Formic Acid, Gradient: 10-100% B over 10 min Detection: 100-600 nm using electrospray ionisation (ESI) positive mode with source temperature 300° C.

Method B (5 min method): LC model: Agilent 1200 (Pump type: Binary Pump, Detector type: DAD) MS model: Agilent G6110A Quadrupole. Column: Xbridge-C18, 2.5 μm, 2.1×30 mm. Column temperature: 30° C. Acquisition of wavelength: 214 nm, 254 nm. Mobile phase: A: 0.07% HCOOH aqueous solution, B: MeOH. Run time: 5 min. MS: Ion source: ES+ (or ES−). MS range: 50˜900 m/z. Fragmentor: 60. Drying gas flow: 10 L/min. Nebulizer pressure: 35 psi. Drying gas temperature: 350° C. Vcap: 3.5 kV.

Preparative Mass-Directed LC

Method A:

Instrument:

Waters ZQ 3100-Mass Detector, Waters 2545-Pump, Waters SFO System Fluidics Organizer, Waters 2996 Diode Array Detector, Waters 2767 Sample Manager

LC Conditions:

Reverse Phase HPLC analysis Column: XBridge TM C18 5 μm 19×50 mm. Injection Volume 500 μL

Solvent A: Water 0.1% Formic Acid. Solvent B:MeCN 0.1% Formic Acid

Gradient: 5% B over 4 min then 5-100% B over 8 min then 100% B over 4 min

Flow rate: 19 mL/min. Detection: 100-600 nm

MS Conditions:

Ion Source: Single-quadrupole. Ion Mode: ES positive. Source Temp: 150° C.

Desolvation Temp: 350° C. Detection: Ion counting. Capillary (KV)-3.00. Cone (V): 30

Extractor (V): 3 RF Lens (V): 0.1 Scan Range: 100-1000 Amu Scan Time: 0.5 sec

Acquisition time: 10 min

Gas Flow:
Desolvation L/hour-650
Cone L/hour-100

Preparative HPLC

Instrument type:

VARIAN 940 LC. Pump type: Binary Pump. Detector type: PDA

LC conditions:

Column: Waters SunFire prep C18 OBD, 5 μm, 19×100 mm. Acquisition wavelength: 214 nm, 254 nm. Mobile Phase: A: 0.07% TFA aqueous solution, B: MeOH, 0.07% TFA.

NMR

Nuclear magnetic resonance (¹H NMR, 600 MHz or 400 MHz) spectra were obtained at 300 K with the CDCl₃ as the solvent, unless otherwise indicated. Chemical shifts are reported in ppm on the 5 scale and referenced to the appropriate solvent peak.

Synthesis of Intermediate A

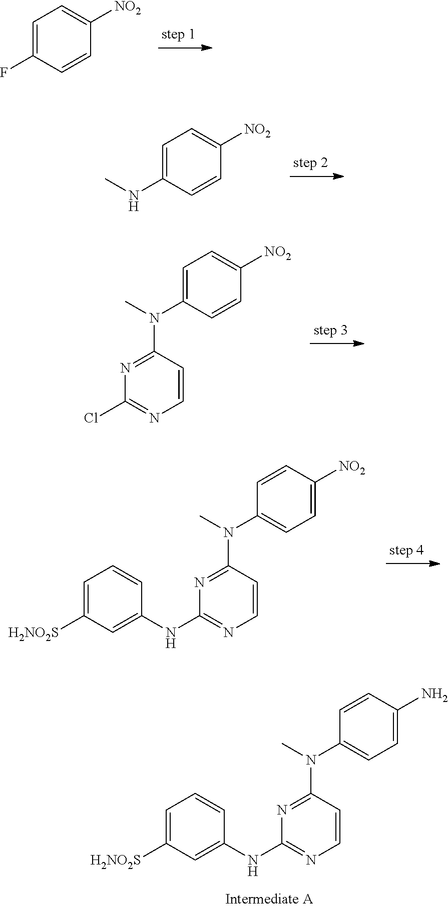 [SMILES: O=[N+]([O-])c1ccc(F)cc1 ; CNc1ccc([N+](=O)[O-])cc1 ; CN(c1ccc([N+](=O)[O-])cc1)c1ccnc(Cl)n1 ; CN(c1ccc([N+](=O)[O-])cc1)c1ccnc(Nc2cccc(S(N)(=O)=O)c2)n1 ; CN(c1ccc(N)cc1)c1ccnc(Nc2cccc(S(N)(=O)=O)c2)n1]

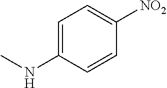

### Step 1: N-methyl-4-nitrobenzenamine

To a solution of 1-fluoro-4-nitrobenzene (50.0 g, 354 mmol) in DMSO (200 mL) were added methanamine hydrochloride (47.1 g, 709 mmol) and potassium carbonate (98.0 g, 709 mmol). The resulting mixture was stirred overnight at 70° C. under nitrogen atmosphere. TLC analysis indicated that the reaction was complete. The mixture was poured into water to give a precipitate which was filtered off and then washed with additional water and dried to yield desired product as yellow solid (50 g, 93%). LCMS (Method B): 1.63 min [MH]⁺=153.1, [MNa]⁺=175.1.

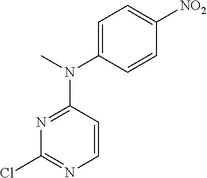 [SMILES: CN(c1ccc([N+](=O)[O-])cc1)c1ccnc(Cl)n1]

### Step 2: 2-chloro-N-methyl-N-(4-nitrophenyl)pyrimidin-4-amine

To a solution of N-methyl-4-nitrobenzenamine (30.0 g, 197 mmol) in DMF (150 mL) was added 2,4-dichloropyrimidine (29.4 g, 197 mmol) and potassium carbonate (40.88 g, 296 mmol). The resulting mixture was stirred at 80° C. overnight. The mixture was diluted with ethyl acetate (300 mL) and water (200 mL), and the combined organic phases were washed with water, brine, dried over sodium sulfate and concentrated to give a residue, which was purified by column chromatography on silica gel (petroleum ether/ethyl acetate, 10:1 to 3:1) to give the desired product (20 g, 38%) as a yellow solid. LCMS (Method B): 2.34 min [MH]⁺=265.0, 267.0, [MNa]⁺=287.0, 289.0.

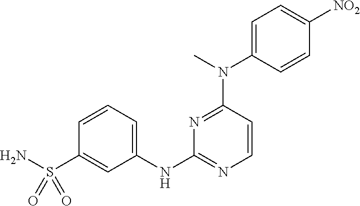 [SMILES: CN(c1ccc([N+](=O)[O-])cc1)c1ccnc(Nc2cccc(S(N)(=O)=O)c2)n1]

### Step 3: 3-((4-(methyl(4-nitrophenyl)amino)pyrimidin-2-yl)amino)benzene sulfonamide

To a solution of 2-chloro-N-methyl-N-(4-nitrophenyl)pyrimidin-4-amine (1.0 g, 3.8 mmol) in 1.4-dioxane (10 mL) was added 3-aminobenzenesulfonamide (651 mg, 3.8 mmol) and cone HCl (0.5 mL). The resulting mixture was stirred at 160° C. for two hours under microwave. LCMS and TLC analysis indicated that the reaction was complete. The solvent was removed under reduced pressure to give a residue. The residue was dissolved into NaOH aqueous solution (4M) and DCM. The organic layer was washed with water, brine, dried over sodium sulfate and concentrated to give a residue, which was purified by column chromatography on silica gel (DCM/MeOH, 30:1 to 10:1) to give the desired product (1 g, 67%) as a yellow solid. LCMS (Method B): 0.96 min [MH]⁺=401.1.

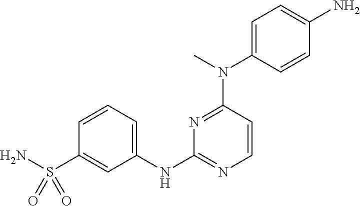 [SMILES: CN(c1ccc(N)cc1)c1ccnc(Nc2cccc(S(N)(=O)=O)c2)n1]

### Step 4: (Intermediate A) 3-((4-(methyl(4-(3-(4-(trifluoromethoxy) phenyl)ureido) phenyl)amino)pyrimidin-2-yl)amino)benzenesulfonamide

To a solution of 3-((4-(methyl(4-nitrophenyl)amino)pyrimidin-2-yl)amino) benzenesulfonamide (800 mg, 2.0 mmol) in EtOH (20 mL) was added zinc (1.3 g, 20 mmol) and NH₄Cl (aq 30 ml). The reaction mixture was stirred at 90° C. for two hours. LCMS and TLC analysis indicated that the reaction was complete. The mixture was filtered off and the liquid was concentrated in vacuo to remove EtOH then the mixture was filtered off to give the desired product (320 mg, 43%) as a white solid. ¹H NMR (400 MHz, DMSO-d₆): δ ppm 3.34 (s, 3H), 5.22 (s, 2H), 5.68 (d, J=6.0 Hz, 1H), 6.65 (d, J=8.4 Hz, 2H), 6.95 (d, J=8.4 Hz, 2H), 7.23 (s, 2H), 7.41 (m, 2H), 7.81 (m, 2H), 8.57 (s, 1H), 8.42 (s, 1H). LCMS (Method B): 0.37 min [MH]⁺=370.1.

Synthesis of Intermediate B

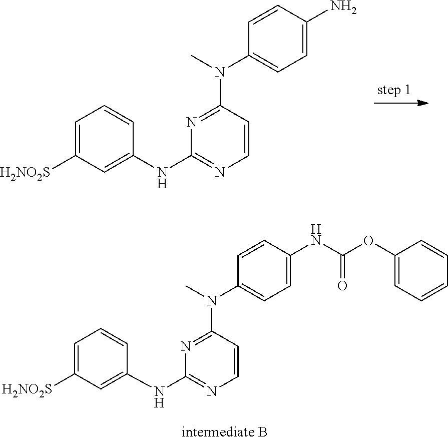 [SMILES: CN(c1ccc(N)cc1)c1ccnc(Nc2cccc(S(N)(=O)=O)c2)n1 ; CN(c1ccc(NC(=O)Oc2ccccc2)cc1)c1ccnc(Nc2cccc(S(N)(=O)=O)c2)n1]

### Step 1: (Intermediate B) 3-(4-(methyl(4-(3-phenylureido)phenyl)amino) pyrimidin-2-ylamino)benzenesulfonamide

To a bottom flask, 3-((4-((4-aminophenyl)(methyl)amino)pyrimidin-2-yl)amino) benzene sulfonamide (intermediate A (1.5 g, 4.1 mmol) was dissolved in THF (20 mL). Pyridine (320 mg, 12.2 mmol) was then added. Phenylchloroformate (698 mg, 4.5 mmol) was added to the mixture slowly. The reaction mixture was stirred at room temperature overnight. The solvent was removed. The crude was washed with water (2×50 mL), diethyl ether (2×50 mL) and dried to give phenyl(4-(methyl(2-((3-sulfamoylphenyl)amino)pyrimidin-4-yl)amino)phenyl) carbamate (1.5 g, 76%) as a yellow solid. ¹H NMR (400 MHz, MeOD-d₄): δ ppm 3.54 (s, 3H), 5.87 (d, J=6.4 Hz, 1H), 7.28 (m, 5H), 7.45 (m, 5H), 7.68 (m, 4H), 7.82 (d, J=6.4 Hz, 1H), 8.62 (br s, 1H). LCMS (Method B): 2.17 [M+H]⁺=491.2.

Synthesis of Intermediate C

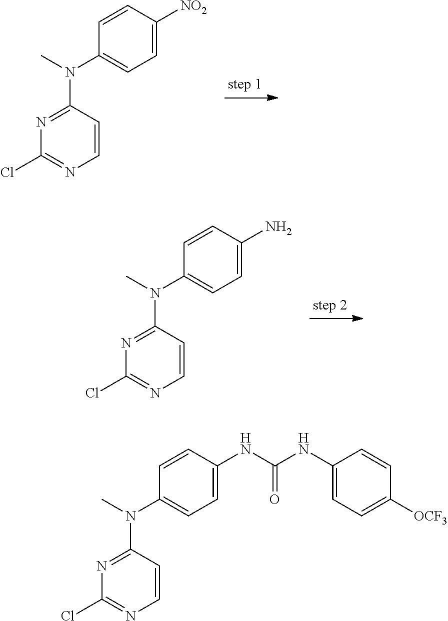 [SMILES: CN(c1ccc([N+](=O)[O-])cc1)c1ccnc(Cl)n1 ; CN(c1ccc(N)cc1)c1ccnc(Cl)n1 ; CN(c1ccc(NC(=O)Nc2ccc(OC(F)(F)F)cc2)cc1)c1ccnc(Cl)n1]

### Step 1: N1-(2-chloropyrimidin-4-yl)-N1-methylbenzene-1,4-diamine

2-Chloro-N-methyl-N-(4-nitrophenyl)pyrimidin-4-amine (from step 2 of preparation of intermediate A, 100 mg, 0.38 mmol) was dissolved in methanol (10 mL) and aq. NH₄Cl (10 mL). Zinc (powder, 245 mg, 3.0 mmol) was added. The reaction mixture was stirred at room temperature overnight. The mixture was concentrated under reduced pressure. The residue was extracted with EtOAc (3×20 mL). The combined organic layers were washed with brine (20 mL), dried over Na₂SO₄. The solvent was removed under reduced pressure to give N1-(2-chloropyrimidin-4-yl)-N1-methylbenzene-1,4-diamine (80 mg, 90%) as a yellow solid which was used in next step directly. LCMS (Method B): 1.10 [M+H]⁺=235.1

### Step 2: (Intermediate C) 1-(4-((2-chloropyrimidin-4-yl) (methyl)amino)phenyl)-3-(4-(trifluoromethoxy)phenyl)urea

To a bottom flask, N1-(2-chloropyrimidin-4-yl)-N1-methylbenzene-1,4-diamine (776 mg, 3.31 mmol) was dissolved in DCM (4 mL). 1-isocyanato-4-(trifluoromethoxy)benzene (672 mg, 3.31 mmol) was added to the mixture. The reaction mixture was stirred at room temperature overnight. The white solid was filtered off and washed with DCM (20 mL), and dried to give 1-(4-((2-chloropyrimidin-4-yl) (methyl)amino)phenyl)-3-(4-(trifluoromethoxy)phenyl)urea (1.06 g, 73%) as a white solid. ¹H NMR (400 MHz, DMSO-d₅): δ ppm 3.40 (s, 3H), 6.27 (d, J=6.0 Hz, 1H), 7.31 (m, 4H), 7.60 (m, 4H), 7.98 (d, J=6.0 Hz, 1H), 8.90 (s, 1H), 8.93 (s, 1H). LCMS (Method B): 3.09 [M+H]⁺=438.2.

Synthesis of Intermediate D and E

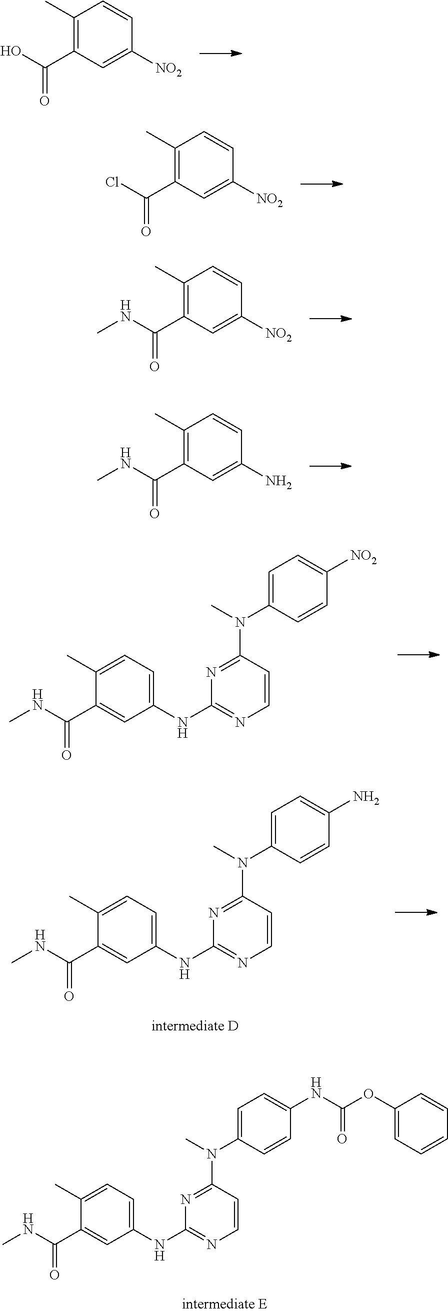

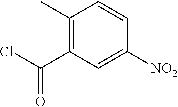

Step 1:

To a solution of 2-methyl-5-nitrobenzoic acid (3.0 g, 16.6 mmol) in thionyl chloride (15 mL) kept under nitrogen atmosphere, was added N,N-dimethylformamide (1 drop). The mixture was heated to reflux for 4 h. The mixture was diluted with dichloromethane (20 mL) and concentrated to dryness to give a white solid (3.3 g, quantitative).

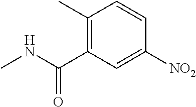

Step 2:

To a solution of 2-methyl-5-nitrobenzoyl chloride (1.1 g, 5.5 mmol) in dichloromethane (20 mL) at 0° C. were added methylamine (411 mg, 6.08 mmol) and triethylamine (1.67 g, 16.6 mmol). The mixture was stirred at room temperature overnight and then washed with water (30 mL) and brine (20 mL), dried over sodium sulfate and concentrated to give a white solid (800 mg, 75%). LCMS (acidic, 5 min): 2.58 min [MH]+=195.0.

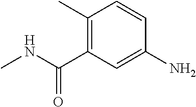

### Step 3: 5-amino-N,2-d imethylbenzamide

To a solution of N,2-dimethyl-5-nitrobenzamide (350 mg, 1.80 mmol) in methanol (20 mL) were added zinc powder (1.5 g) and saturated ammonium chloride aqueous solution (20 mL). The mixture was stirred at room temperature overnight. The solid was filtered off and the solvent methanol was removed under reduced pressure to give a residue which was partitioned between water and ethylacetate. The organic phase was separated and washed with water, brine, dried over sodium sulfate and concentrated under reduced pressure to give 5-amino-N,2-dimethylbenzamide (250 mg, 85%) as a light yellow solid. LCMS (Method B): 2.96 min [MH]⁺=165.2.

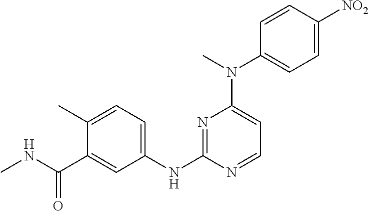 [SMILES: CNC(=O)c1cc(Nc2nccc(N(C)c3ccc([N+](=O)[O-])cc3)n2)ccc1C]

### Step 4: N,2-dimethyl-5-((4-(methyl(4-nitrophenyl)amino)pyrimidin-2-yl)amino) benzamide

To a solution of 5-amino-N,2-dimethylbenzamide (250 mg, 1.52 mmol) in 1,4-dioxane (10 mL) were added 2-Chloro-N-methyl-N-(4-nitrophenyl)pyrimidin-4-amine (from step 2 of preparation of intermediate A, 403 mg, 1.52 mmol) and p-toluenesulfonic acid monohydrate (230 mg, 1.22 mmol). The mixture was stirred at 120° C. for 3 hours. The solvent was removed under reduced pressure to give a residue which was then treated with saturated aqueous ammonia solution (30 mL) to form a precipitate. The solid was filtered and dried under reduced pressure to give N,2-dimethyl-5-((4-(methyl(4-nitrophenyl)amino)pyrimidin-2-yl)amino)benzamide (500 mg, 87%) as a brown solid. LCMS (Method B): 2.55 min [MH]⁺=393.1.

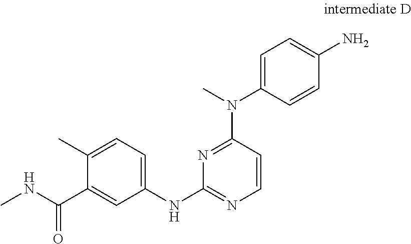 [SMILES: CNC(=O)c1cc(Nc2nccc(N(C)c3ccc(N)cc3)n2)ccc1C]

### Step 5: (intermediate D) 5-((4-((4-aminophenyl)(methyl)amino)pyrimidin-2-yl)amino)-N,2-dimethyl benzamide

To a solution of 5-(4-(N-methyl-N-(4-nitrophenyl)amino) pyrimidin-2-ylamino)-N,2-dimethylbenzamide (200 mg, 0.51 mmol) in methanol (30 mL) were added zinc powder (1.0 g) and saturated ammonium chloride aqueous solution (30 mL). The mixture was stirred at room temperature overnight. The solid was filtered off and the solvent methanol was removed under reduced pressure to give a residue which was partitioned between water and ethylacetate. The organic phase was separated and washed with water, brine, dried over sodium sulfate and concentrated under reduced pressure to give 5-((4-((4-aminophenyl)(methyl)amino)pyrimidin-2-yl)amino)-N,2-dimethyl benzamide (150 mg, 81%) as a light yellow solid. LCMS (Method B): 1.63 min [MH]⁺=363.1.

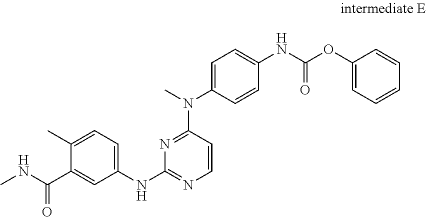 [SMILES: CNC(=O)c1cc(Nc2nccc(N(C)c3ccc(NC(=O)Oc4ccccc4)cc3)n2)ccc1C]

### Step 6: (intermediate E) phenyl(4-((2-((3-(dimethylcarbamoyl)-4-methylphenyl) amino)pyrimidin-4-yl)(methyl)amino)phenyl) carbamate

5-((4-((4-aminophenyl)(methyl)amino)pyrimidin-2-yl)amino)-N,2-dimethyl benzamide (1.78 g, 4.92 mmol) and DIEA (1.2 g, 9.38 mmmol) were dissolved in THF (60 mL), followed by a slow addition of phenylchloroformate (771 mg, 4.92 mmol). The reaction mixture was stirred at room temperature overnight. The solvent was removed and the crude was purified by column chromatography (DCM/methanol, 100:0 to 100:2) to give phenyl (4-((2-((3-(dimethylcarbamoyl)-4-methylphenyl)amino)pyrimidin-4-yl)(methyl)amino) phenyl)carbamate (1.1 g, 48.7%) as a yellow solid. LCMS (Method B): 2.28 min [MH]⁺=483.2. ¹H NMR (400 MHz, DMSO-d₆) δ ppm 2.28 (s, 3H) 2.75 (d, J=4.4 Hz, 3H) 3.47 (s, 3H) 5.77(brs, 1H), 7.19 (d, J=8.4 Hz, 1H) 7.24-7.30 (m, 3H) 7.38 (d, J=8.8 Hz, 2H) 7.36-7.48 (m, 2H) 7.56 (d, J=8.4 Hz, 1H) 7.65-7.67 (m, 3H) 7.89 (d, J=6.8 Hz, 1H) 8.21 (br s, 1H) 10.35 (br s, 1H) 10.50 (br s, 1H).

Synthesis of Intermediate F and G

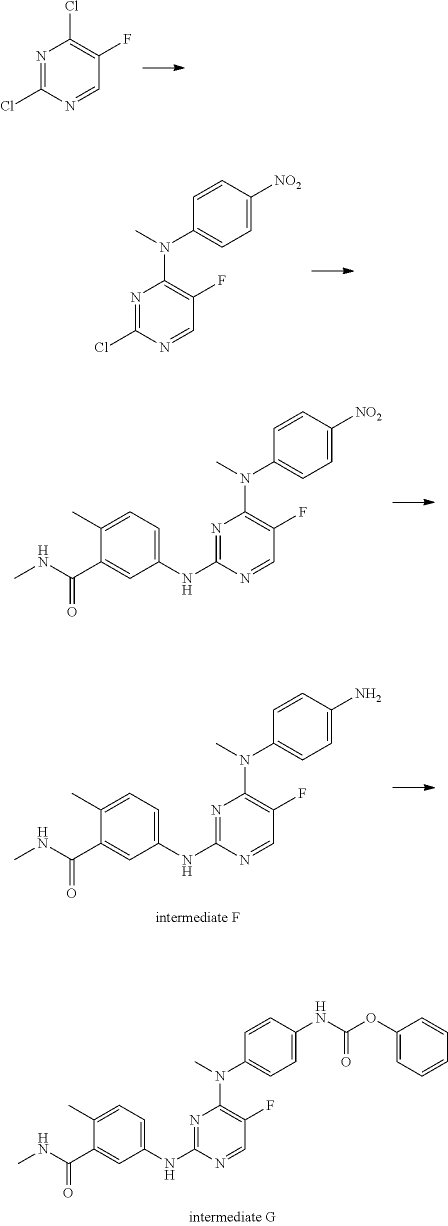 [SMILES: Fc1cnc(Cl)nc1Cl ; CN(c1ccc([N+](=O)[O-])cc1)c1nc(Cl)ncc1F ; CNC(=O)c1cc(Nc2ncc(F)c(N(C)c3ccc([N+](=O)[O-])cc3)n2)ccc1C ; CNC(=O)c1cc(Nc2ncc(F)c(N(C)c3ccc(N)cc3)n2)ccc1C ; CNC(=O)c1cc(Nc2ncc(F)c(N(C)c3ccc(NC(=O)Oc4ccccc4)cc3)n2)ccc1C]

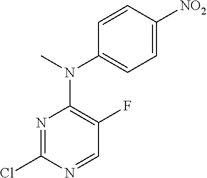 [SMILES: CN(c1ccc([N+](=O)[O-])cc1)c1nc(Cl)ncc1F]

### Step 1: 2-chloro-5-fluoro-N-methyl-N-(4-nitrophenyl)pyrimidin-4-amine

To a flask were added N-methyl-4-nitroaniline (760 mg, 5.0 mmol), 2,4-dichloro-5-fluoropyrimidine (835 mg, 5.0 mmol), cesium carbonate (2.44 g, 7.5 mmol) and DMF (15 mL). The mixture was stirred at 60° C. overnight. The mixture was partitioned between ethylacetate and water. The aqueous layer was extracted with ethylacetate several times. The combined organic layers were washed with brine, dried over sodium sulfate to give a residue which was purified by column chromatography (petroleum ether/ethylacetate,15:1) to give 2-chloro-5-fluoro-N-methyl-N-(4-nitrophenyl)pyrimidin-4-amine (530 mg, 38%) as a yellow solid. LCMS (Method B): 2.07 min [MH]⁺=283.0.

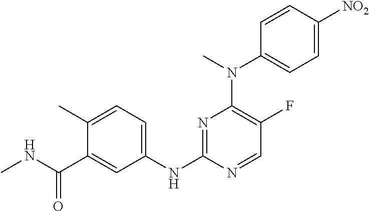 [SMILES: CNC(=O)c1cc(Nc2ncc(F)c(N(C)c3ccc([N+](=O)[O-])cc3)n2)ccc1C]

### Step 2: 5-((5-fluoro-4-(methyl(4-nitrophenyl)amino)pyrimidin-2-yl) amino)-N,2-dimethyl benzamide

To a solution of 2-chloro-5-fluoro-N-methyl-N-(4-nitrophenyl)pyrimidin-4-amine (1.3 g, 4.60 mmol), 5-amino-N,2-dimethylbenzamide (from step 2 of preparation of intermediate A, 775 mg, 4.60 mmol) in isopropanol (30 mL) was added concentrated HCl (0.2 mL). The resulting mixture was stirred at 85° C. overnight under nitrogen atmosphere, cooled to RT and diluted with NaOH 1M, water and ethyl acetate. The organic layer was separated and the aqueous layer was extracted with ethyl acetate. The combined organic layers were washed with water, brine, dried over sodium sulfate and concentrated under reduced pressure. The residue was purified by column chromatography (dichloromethane/methanol=100/1 to 60/1) to yield 5-((5-fluoro-4-(methyl(4-nitrophenyl)amino)pyrimidin-2-yl) amino)-N,2-dimethyl benzamide (640 mg, 52%) as a yellow solid. LCMS (method B): 2.55 min [MH]+=411.2.

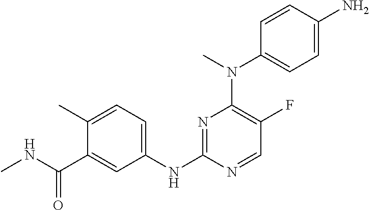 [SMILES: CNC(=O)c1cc(Nc2ncc(F)c(N(C)c3ccc(N)cc3)n2)ccc1C]

### Step 3: (intermediate F) 5-((4-((4-aminophenyl)(methyl)amino)-5-fluoropyrimidin-2-yl) amino)-N,2-dimethylbenzamide

To a solution of 5-((5-fluoro-4-(methyl(4-nitrophenyl)amino)pyrimidin-2-yl)amino)-N,2-dimethylbenzamide (540 mg, 1.31 mmol) in DMF (20 mL) was added zinc powder (855 mg, 13.1 mmol) and ammonium chloride aqueous solution (20 mL). The resulting mixture was stirred at 50° C. overnight, cooled to RT and diluted with aqueous NaHCO3, water and ethyl acetate. The organic layer was separated and the aqueous layer was extracted with ethyl acetate. The combined organic layers were washed with water, brine, dried over sodium sulfate and concentrated under reduced pressure. The residue was purified by column chromatography (eluent: dichloromethane/methanol=60/1-40/1) to give 5-((4-((4-aminophenyl)(methyl)amino)-5-fluoropyrimidin-2-yl)amino)-N,2-dimethyl benzamide (580 mg, 98%) as a pale-yellow solid. LCMS (method B): 0.525 min [MH]+=381.2.

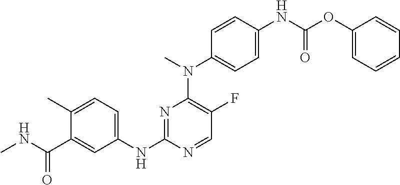 [SMILES: CNC(=O)c1cc(Nc2ncc(F)c(N(C)c3ccc(NC(=O)Oc4ccccc4)cc3)n2)ccc1C]

### Step 4: (intermediate G) phenyl (4-((5-fluoro-2-((4-methyl-3-(methylcarbamoyl) phenyl)amino)pyrimidin-4-yl)(methyl)amino)phenyl)carbamate

To a solution of 5-((4-((4-aminophenyl)(methyl)amino)-5-fluoropyrimidin-2-yl) amino)-N,2-dimethylbenzamide (454 mg, 1.19 mmol) in THF (12 mL) was added pyridine (188 mg, 2.38 mmol), followed by a dropwise addition of phenyl chloroformate (187 mg, 1.19 mmol) at rt. The resulting mixture was stirred at rt for 2 h under nitrogen atmosphere, diluted with an aqueous solution of HCl 1M, water and ethyl acetate. The organic layer was separated and the aqueous layer was extracted with ethyl acetate. The combined organic layers were washed with water, brine, dried over sodium sulfate and concentrated under reduced pressure to give the titled compound (600 mg, 100%) as a yellow solid. ¹H NMR (400 MHz, DMSO-d6): δ ppm 2.24 (s, 3H), 2.74 (d J=4.8 Hz, 3H), 3.44 (s, 3H), 7.09 (d J=8.4 Hz, 1H), 7.29 (m, 5H), 7.47 (t, J=8.0 Hz, 2H), 7.54 (d, J=8.4 Hz, 2H), 7.61 (dd, J=8.0, 2.0 Hz, 1H), 7.81 (d, J=2.0 Hz, 1H), 7.97 (d, J=5.6 Hz, 1H), 8.08 (m, 1H), 9.29 (s, 1H), 10.33 (s, 1H). LCMS (method B): 2.54 min [MH]+=501.2.

Synthesis of Intermediate H

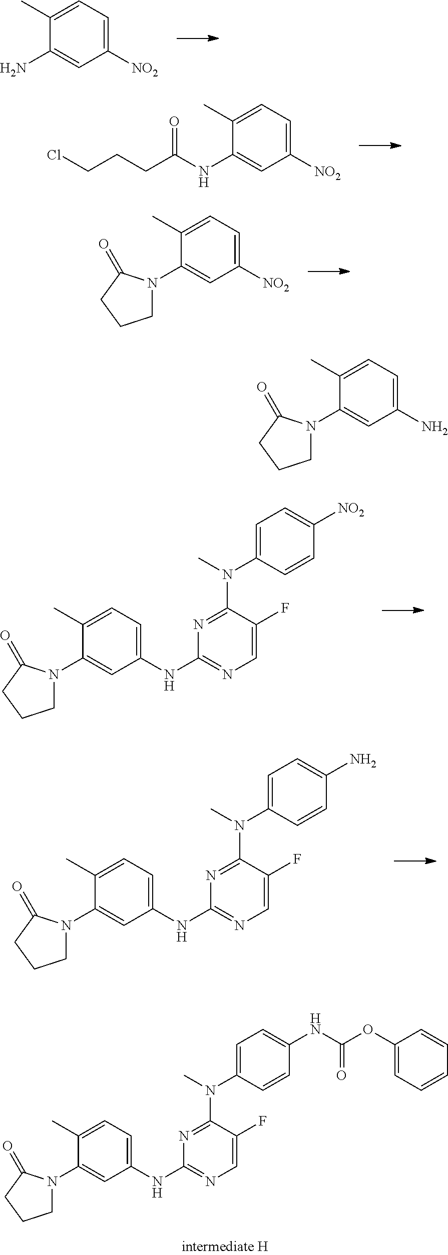

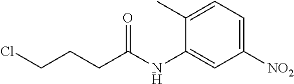 [SMILES: Cc1ccc([N+](=O)[O-])cc1NC(=O)CCCCl]

### Step 1: 4-chloro-N-(2-methyl -5-nitrophenyl)butanamide

To a solution of 2-methyl-5-nitroaniline (5.0 g, 32.9 mmol) in DCM (50 mL) was added triethylamine (3.99 g, 39.4 mmol) and 4-chlorobutanoyl chloride (5.1 g, 36.1 mmol) at 0° C. The solution was stirred at room temperature for 4 hours under N₂. The mixture was diluted with DCM (120 mL) and water (50 mL). The organic layer was separated and the aqueous layer was extracted with DCM. The combined organic phases were washed with 0.5 M HCl, water, brine, dried over sodium sulfate and concentrated to give a residue which was purified by column chromatography on silica gel (DCM/MeOH, 300:1) to give 4-chloro-N-(2-methyl-5-nitrophenyl)butanamide (6.9 g, 82%) as pale-gray solid LCMS (Method B): 2.20 min [MH]⁺=257.1/259.1, [MNa]⁺=279.1/281.1

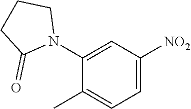 [SMILES: Cc1ccc([N+](=O)[O-])cc1N1CCCC1=O]

### Step 2: 1-(2-methyl-5-nitrophenyl)pyrrolidin-2-one

To a solution of 4-chloro-N-(2-methyl-5-nitrophenyl)butanamide (500 mg, 1.95 mmol) in 6% aqueous NaOH/i-PrOH (1:1, 20 mL) was stirred at RT for 3 h under N₂. The mixture was diluted with ethylacetate (50 mL) and water (20 mL). The organic layer was separated and the aqueous layer was extracted with ethylacetate. The combined organic phases were washed with water, brine, dried over sodium sulfate and concentrated to give a residue which was purified by column chromatography on silica gel (DCM/MeOH, 250:1 to 200:1) to give 1-(2-methyl-5-nitrophenyl)pyrrolidin-2-one (214 mg, 52%) as pale-yellow solid. LCMS (Method B): 1.23 min [MH]⁺=221.1, [MNa]⁺=243.1, [2MH]⁺=441.2, [2MNa]⁺=463.1.

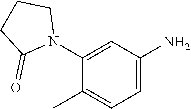

### Step 3: 1-(5-amino-2-methylphenyl)pyrrolidin-2-one

To a solution of 1-(2-methyl-5-nitrophenyl)pyrrolidin-2-one (214 mg, 0.97 mmol) in EtOAC (10 mL) was added 10% Pd/C (30 mg). The reaction mixture was stirred under an hydrogen atmosphere overnight at RT. The catalyst was removed by filtration and the solvent was removed under reduced pressure to give a residue which was purified by column chromatography on silica gel (DCM/MeOH, 50:1) to give 1-(5-amino-2-methylphenyl)pyrrolidin-2-one (142 mg, 77%) as a white solid. LCMS (Method B): 0.52 min [MH]⁺=191.1, [2MH]⁺=381.2, [2MNa]⁺=403.2.

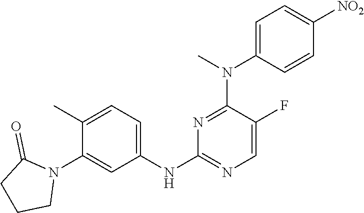 [SMILES: Cc1ccc(Nc2ncc(F)c(N(C)c3ccc([N+](=O)[O-])cc3)n2)cc1N1CCCC1=O]

### Step 4: 1-(5-(5-fluoro-4-(methyl(4-nitrophenyl)amino)pyrimidin-2-ylamino)-2-methylphenyl)pyrrolidin-2-one

To a solution of 2-chloro-5-fluoro-N-methyl-N-(4-nitrophenyl)pyrimidin-4-amine (1.0 g, 3.54 mmol) in iso-propanol (10 mL) were added 1-(5-amino-2-methylphenyl)pyrrolidin-2-one (673 mg, 3.8 mmol) and conc HCl (0.5 mL). The resulting mixture was stirred at 85° C. for 16 hours. The solvent was removed under reduced pressure to give a residue. The residue was dissolved into aqueous NaOH solution (4 M) and DCM. The organic layer was separated, washed with water, brine, dried (over sodium sulfate) and concentrated under reduced pressure to give a residue which was purified by column chromatography on silica gel (eluent DCM/MeOH, 99:1 to 96:4) to give 1-(5-(5-fluoro-4-(methyl(4-nitrophenyl)amino)pyrimidin-2-ylamino)-2-methylphenyl) pyrrolidin-2-one (1.0 g, 65%) as a yellow solid. LCMS (method B): 2.70 min [MH]⁺=437.2.

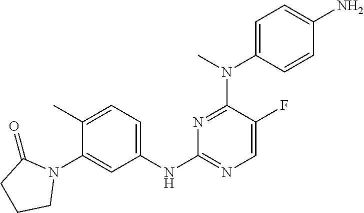 [SMILES: Cc1ccc(Nc2ncc(F)c(N(C)c3ccc(N)cc3)n2)cc1N1CCCC1=O]

### Step 5: 1-(5-(4-((4-aminophenyl)(methyl)amino)-5-fluoropyrimidin-2-ylamino)-2-methylphenyl)pyrrolidin-2-one

To a solution of 1-(5-(5-fluoro-4-(methyl(4-nitrophenyl)amino)pyrimidin-2-ylamino)-2-methylphenyl) pyrrolidin-2-one (1 g, 2.29 mmol) in methanol (20 mL) was added zinc (1.5 g, 22.9 mmol) and aqueous NH₄Cl solution(10 mL). The reaction mixture was stirred at room temperature for 16 hours. The solids were removed by filtration and the methanol layer was concentrated under reduced pressure to afford a precipitate. The resulting precipitate was collected by filtration and washed with water to give 1-(5-(4-((4-aminophenyl) (methyl)amino)-5-fluoropyrimidin-2-ylamino)-2-methylphenyl)pyrrolidin-2-one (600 mg, 64.5%) as a white solid. LCMS (method B): 1.85 min [MH]⁺=407.2

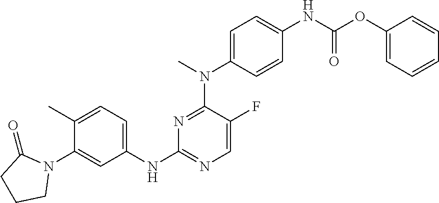 [SMILES: Cc1ccc(Nc2ncc(F)c(N(C)c3ccc(NC(=O)Oc4ccccc4)cc3)n2)cc1N1CCCC1=O]

### Step 6 (Intermediate H): phenyl 4-((5-fluoro-2-(4-methyl-3-(2-oxopyrrolidin-1-yl)phenylamino) pyrimidin-4-yl) (methyl)amino)phenylcarbamate

1-(5-(4-((4-aminophenyl)(methyl)amino)-5-fluoropyrimidin-2-ylamino)-2-methylphenyl)pyrrolidin-2-one (950 mg, 2.34 mmol) was dissolved in THF (50 mL). Pyridine (554 mg, 7.01 mmol) followed by phenylchloroformate (403 mg, 2.574 mmol) were added slowly. The reaction mixture was stirred at room temperature for 16 hours and concentrated under reduced pressure. The crude solid was washed with water (2×50 mL), ether (2×50 mL), dried under reduced pressure to give phenyl 4-((5-fluoro-2-(4-methyl-3-(2-oxopyrrolidin-1-yl)phenylamino) pyrimidin-4-yl) (methyl)amino)phenylcarbamate (860 mg, 70%) as a yellow solid. LCMS (method B): 2.66 min [MH]⁺=527.2

¹H NMR (400 MHz, DMSO-d₆) δ ppm 2.07 (s, 3H) 2.14 (m, 2H) 2.42 (d, J=8.0 Hz, 2H) 3.43 (s, 3H) 3.65 (d, J=6.8 Hz, 2H), 7.13 (d, J=8.4 Hz, 1H) 7.23-7.29 (m, 5H) 7.43-7.54 (m, 5H) 7.67 (d, J=2.0 Hz, 1H) 7.97 (d, J=5.6 Hz, 1H) 9.30 (s, 1H) 10.35 (s, 1H).

General Procedure A for the Synthesis of the Ureas:

To a solution of the intermediate A (1 mmol) in dry DMF (0.2 mL) under N₂ was added an isocyanate (1 mmol) dropwise. The reaction mixture was stirred for 16 hours at rt, concentrated under reduced pressure and the residue was purified by preparative mass directed LC to afford the corresponding compound.

General Procedure B for the Synthesis of the Ureas:

To a solution of intermediate B (1 mmol) in dry THF under N₂ was added an arylamine (2 mmol) followed by DIEA (2 mmol). The reaction mixture was heated to 60° C. for 16 hours, concentrated under reduced pressure and the residue was purified by preparative HPLC to afford the corresponding compound.

General Procedure C for the Addition of Aniline to 2-chloropyrimidine:

To a solution of aniline (1 mmol) and intermediate C (1 mmol) in 2-propanol (10 mL) was added a solution of concentrated HCl (2 drops). The reaction mixture was heated to 80° C. for 16 hours. The solvent was removed and the crude product was purified by column chromatography to afford the corresponding compound.

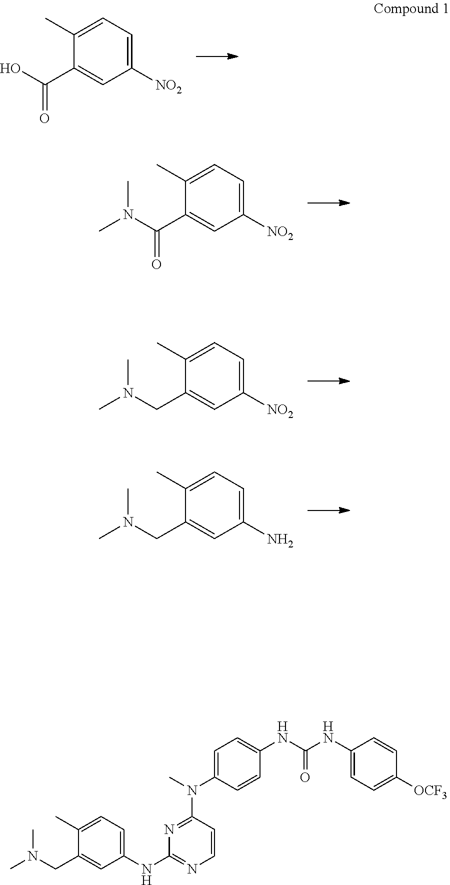 [SMILES: Cc1ccc([N+](=O)[O-])cc1C(=O)O ; Cc1ccc([N+](=O)[O-])cc1C(=O)N(C)C ; Cc1ccc([N+](=O)[O-])cc1CN(C)C ; Cc1ccc(N)cc1CN(C)C ; Cc1ccc(Nc2nccc(N(C)c3ccc(NC(=O)Nc4ccc(OC(F)(F)F)cc4)cc3)n2)cc1CN(C)C]

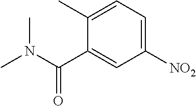

### Step 1: N,N,2-trimethyl-5-nitrobenzamide

2-methyl-5-nitrobenzoic acid (1 g, 5.5 mmol) were dissolved in SOCl₂ (15 ml), followed by addition of DMF (1 drop). The reaction mixture was heated to reflux for 4 h. The excess thionyl chloride was removed under reduced pressure. DCM (3×10 ml) was added and removed to give a white solid. To another bottom flask, Me₂NH.HCl (490 mg, 6.08 mmol) and TEA (1.67 g, 16.6 mmol) were added in DCM (20 mL), followed by addition of 2-methyl-5-nitrobenzoyl chloride (5.5 mmol) in DCM (5 mL) slowly at 0° C. The reaction mixture was stirred at room temperature overnight. The solvent was removed and the crude product was washed with water (3×30 mL), dried to give the desired product (600 mg, 52%) as a white solid. LCMS (method B): 1.06 min [MH]⁺=209.4

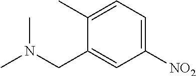

### Step 2: N,N-dimethyl-1-(2-methyl-5-nitrophenyl)methanamine

N,N,2-trimethyl-5-nitrobenzamide (475 mg, 2.28 mmol) was dissolved in THF (15 ml), followed by of BH₃ in THF (1 mol/L, 25 mL) under nitrogen. The reaction mixture was heated to 60° C. for 8 h. TLC and LCMS showed the reaction was complete. Aqueous HCl solution (2 M, 20 mL) was added and the organic layer was extracted with ethyl acetate (3×20 mL). The combined organic layers were washed with water (30 mL), brine, dried over sodium sulfate and concentrated under reduced pressure to give a crude product which was purified by column chromatography (eluent DCM:methanol, 100:0 to 100:2) to give the desired product (320 mg, 72%) as a yellow solid. LCMS (method B): 2.31 min [MH]⁺=195.1

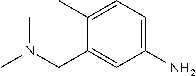

### Step 3 : 3-((dimethylamino)methyl)-4-methylaniline

N,N-dimethyl(2-methyl-5-nitrophenyl)methanamine (320 mg, 1.65 mmol) was dissolved in methanol (30 mL), followed by addtion of 10% wet Pd/C (35 mg). The reaction mixture was stirred under H₂ atmosphere overnight. The catalyst was removed by filtration and the solvent was removed under reduced pressure to give the desired product (200 mg, 74%) as a yellow solid, which was used directly in next step. LCMS (method B): 0.25 min [MH]⁺=165.1

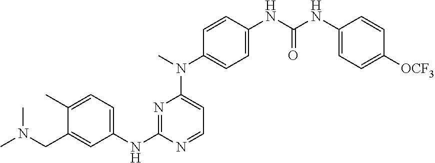 [SMILES: Cc1ccc(Nc2nccc(N(C)c3ccc(NC(=O)Nc4ccc(OC(F)(F)F)cc4)cc3)n2)cc1CN(C)C]

### Step 4: 1-(4-((2-((3-((dimethylamino)methyl)-4-methylphenyl)amino)pyrimidin-4-yl)(methyl)amino)phenyl)-3-(4-(trifluoromethoxy)phenyl)urea

Following general procedure C using intermediate C (140 mg, 0.32 mmol) and 3-((dimethylamino)methyl)-4-methylbenzenamine (53 mg, 0.32 mmol), 1-(4-((2-((3-((dimethylamino)methyl)-4-methylphenyl)amino)pyrimidin-4-yl)(methyl) amino)phenyl)-3-(4-(trifluoromethoxy)phenyl)urea was obtained as a white solid (50 mg, 28%). ¹H NMR (400 MHz, DMSO-d₆): δ 2.32 (s, 3H), 2.62 (s, 6H), 3.41 (s, 3H), 4.00 (br s, 2H), 5.08 (d, J=5.6 Hz, 1H), 7.12 (d, J=8.4 Hz, 1H), 7.31 (m, 4H), 7.61 (m, 5H), 7.79 (s, 1H), 7.87 (d, J=7.0 Hz, 1H), 9.14 (s, 1H), 9.44 (s, 1H), 9.51 (s, 1H). LCMS (Method B): 2.24 min [MH]⁺=566.3.

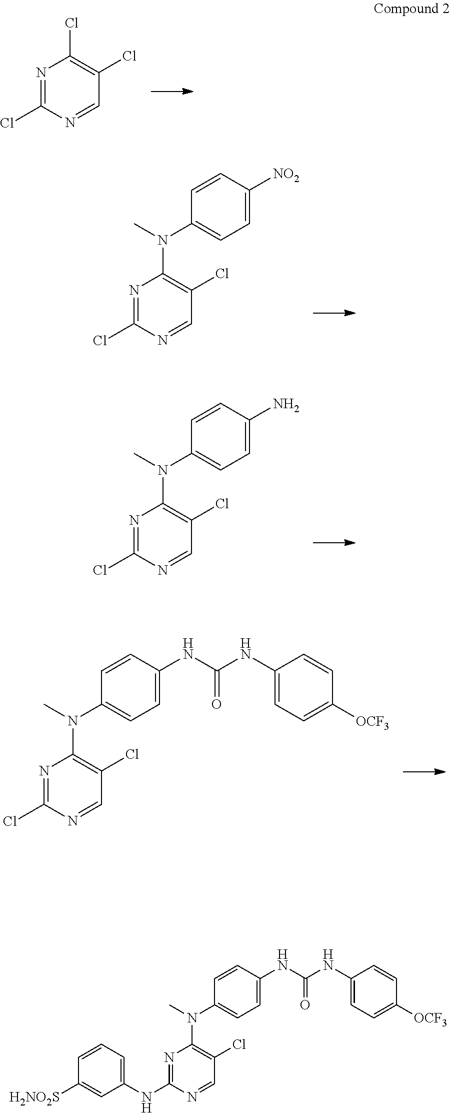 [SMILES: Clc1ncc(Cl)c(Cl)n1 ; CN(c1ccc([N+](=O)[O-])cc1)c1nc(Cl)ncc1Cl ; CN(c1ccc(N)cc1)c1nc(Cl)ncc1Cl ; CN(c1ccc(NC(=O)Nc2ccc(OC(F)(F)F)cc2)cc1)c1nc(Cl)ncc1Cl ; CN(c1ccc(NC(=O)Nc2ccc(OC(F)(F)F)cc2)cc1)c1nc(Nc2cccc(S(N)(=O)=O)c2)ncc1Cl]

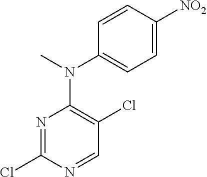 [SMILES: CN(c1ccc([N+](=O)[O-])cc1)c1nc(Cl)ncc1Cl]

### Step 1: 2,5-dichloro-N-methyl-N-(4-nitrophenyl)pyrimidin-4-amine

To a flask were added N-methyl-4-nitroaniline (700 mg, 4.6 mmol), 2,4,5-trichloro-pyrimidine (1.70 g, 9.27 mmol), cesium carbonate (2.26 g, 6.94 mmol) and DMF (20 mL). The mixture was stirred at 90° C. for 4 h. The mixture was partitioned between ethylacetate and water. The aqueous layer was extracted with ethylacetate several times. The combined organic layers were washed with brine, dried over sodium sulfate to give a residue which was purified by column chromatography (petroleum ether/ethylacetate, 20:1) to give 2,5-dichloro-N-methyl-N-(4-nitrophenyl)pyrimidin-4-amine (510 mg, 40%) as a yellow solid. LCMS (Method B): 3.11 min [MH]⁺=299.0.

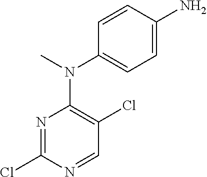 [SMILES: CN(c1ccc(N)cc1)c1nc(Cl)ncc1Cl]

### Step 2: N1-(2,5-dichloroyrimidin-4-yl)-N1-methylbenzene-1,4-diamine

To a mixture of 2,5-dichloro-N-methyl-N-(4-nitrophenyl)pyrimidin-4-amine (140 mg, 0.468 mmol) in methanol (10 mL) was added zinc powder (304 mg, 4.68 mmol) and an saturated ammonium chloride solution (10 mL). The resulting mixture was stirred at room temperature for 16 hours. TLC and LCMS analysis indicated that the product formed and a lot of starting material remained. The mixture was then heated to 60° C. for 3 hours. The zinc powder was filtered off and the filtrate was partitioned between ethylacetate and 1 M sodium hydroxide solution. The aqueous layer was extracted with ethylacetate several times. The combined organic layers were washed with brine, dried over sodium sulfate and concentrated to give N1-(2,5-dichloroyrimidin-4-yl)-N1-methylbenzene-1,4-diamine (120 mg, 95%) as a yellow solid. LCMS (Method B): 2.11 min [MH]⁺=269.0.

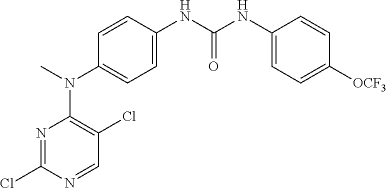 [SMILES: CN(c1ccc(NC(=O)Nc2ccc(OC(F)(F)F)cc2)cc1)c1nc(Cl)ncc1Cl]

### Step 3: 1-(4-((2,5-dichloropyridin-4-yl)(methyl)amino)phenyl)-3-(4-(trifluoro-methoxy)phenyl)urea

A solution of N1-(2,5-dichloropyrimidin-4-yl)-N1-methylbenzene-1,4-diamine (172 mg, 0.68 mmol) in THF (3 mL) was treated with 1-isocyanato-4-(trifluoromethoxy)benzene (95 mg, 0.468 mmol). The reaction mixture was stirred at room temperature overnight. The reaction mixture was then partitioned between ethylacetate and water. The aqueous layer was extracted with ethylacetate several times. The combined organic layers were concentrated to give a residue which was purified by column chromatography (petroleum ether/ethylacetate, 5:1 to 3:1) to give 1-(4-((2,5-dichloropyridin-4-yl)(methyl)amino) phenyl)-3-(4-(trifluoromethoxy)phenyl)urea (120 mg, 57%) as a white solid. LCMS (Method B): 3.33 min [MH]⁺=472.1.

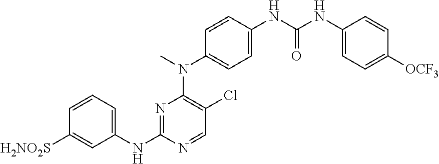 [SMILES: CN(c1ccc(NC(=O)Nc2ccc(OC(F)(F)F)cc2)cc1)c1nc(Nc2cccc(S(N)(=O)=O)c2)ncc1Cl]

### Step 4: 3-((5-Chloro-4-(methyl(4-(3-(4-(trifluoromethoxy) phenyl)ureido) phenyl)amino) pyrimidin-2-yl) amino) benzenesulfonamide.

To a sealed tube were added 1-(4-((2,5-dichloro-pyridin-4-yl)(methyl) amino)phenyl)-3-(4-(trifluoromethoxy)phenyl)urea (50 mg, 0.106 mmol), 3-aminophenylsulfamide (18 mg, 0.106 mmol), concentrated HCI solution (2 drops) and isopropanol (2 mL). The resulting mixture was stirred at 90° C. for 16 hours. The solvent was removed and the residue was purified by prep HPLC to give 3-((5-chloro-4-(methyl(4-(3-(4-(trifluoromethoxy)phenyl)ureido)phenyl) amino)pyrimidin-2-yl)amino)benzenesulfonamide (20 mg, 31%) as a white solid. ¹H NMR (400 MHz, DMSO-d₆): δ ppm 3.46 (s, 3H), 7.16 (d, J=8.8 Hz, 2H), 7.31 (m, 4H), 7.41 (d, J=8.0 Hz, 1H), 7.48 (m, 3H), 7.58 (d, J=7.2 Hz, 2H), 7.79 (d, J=8.4 Hz, 1H), 8.06 (s, 1H), 8.54 (s, 1H), 8.91(s, 1H), 8.99 (s, 1H), 9.87 (s, 1H). LCMS (Method B): 2.98 min [MH]⁺=608.1, [MNa]⁺=630.1.

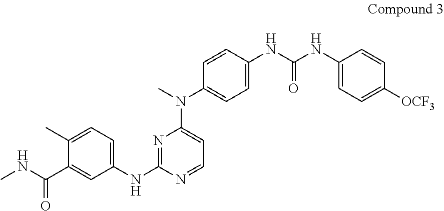 [SMILES: CNC(=O)c1cc(Nc2nccc(N(C)c3ccc(NC(=O)Nc4ccc(OC(F)(F)F)cc4)cc3)n2)ccc1C]

N-2-Dimethyl-5-((4-(methyl(4-(3-(4-(trifluoromethoxy)phenyl)ureido)phenyl) amino) pyrimidin-2-yl)amino)benzamide.

To a solution of intermediate D (150 mg, 0.41 mmol) in THF (15 mL) were added 4-(trifluoromethoxy)phenyl isocyanate (91 mg, 0.45 mmol) and N,N-diisopropylethylamine (106 mg, 0.82 mmol). The mixture was stirred at room temperature overnight. The reaction mixture was concentrated under reduced pressure to give a residue which was purified by silica-gel chromatography (dichloromethane/methanol, 25:1) to give N-2-dimethyl-5-((4-(methyl(4-(3-(4-(trifluoromethoxy)phenyl)ureido)phenyl)amino)pyrimidin-2-yl)amino)benzamide (90 mg, 40%) as a white solid. NMR (400 MHz, DMSO-d₆): δ ppm 2.23 (s, 3H), 2.73 (d, J=4.4 Hz, 3H), 3.40 (s, 3H), 5.75 (d, J=6.0 Hz, 1H), 7.06 (d, J=8.4 Hz, 1H), 7.26 (d, J=8.4 Hz, 2H), 7.30 (d, J=8.4 Hz, 2H), 7.57 (m, 4H), 7.65 (dd, J=8.4 and 2.0 Hz, 1H), 7.90-7.81 (m, 2H), 8.09 (dd, J=8.8 and 4.0 Hz, 1H), 8.90 (s, 1H), 8.96 (s, 1H), 9.19 (s, 1H). LCMS (Method B): 2.55 min [MH]⁺=566.2.

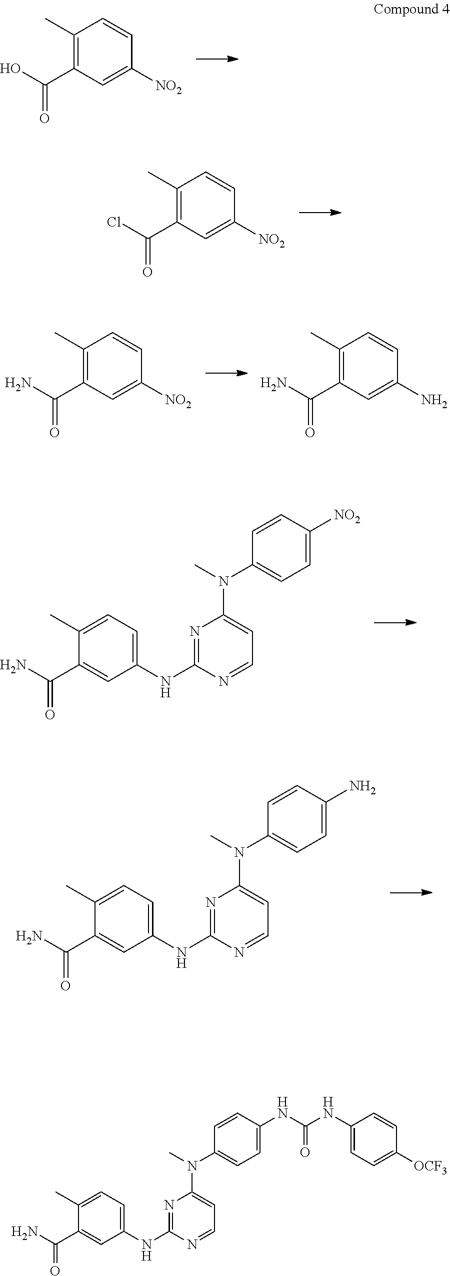

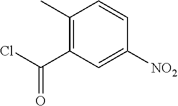

### Step 1: 2-methyl-5-nitrobenzoyl chloride

To a solution of 2-methyl-5-nitrobenzoic acid (3 g, 16.6 mmol) in thionyl chloride (15 mL) was added DMF (1 drop), and the resultant mixture was stirred at 70° C. for 4 hours. The excess thionyl chloride was removed on a rotary evaporator to give 2-methyl-5-nitrobenzoyl chloride (3.3 g, quantitative) as a white solid.

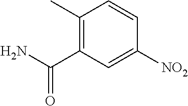

### Step 2: 2-methyl-5-nitrobenzamide

A solution of 2-methyl-5-nitrobenzoyl chloride (1.1 g, 5.53 mmol) was added to saturated ammonia aqueous solution (20 mL) at room temperature and the reaction mixture was stirred 5 hours at room temperature. The solid was collected via filtration to give 2-methyl-5-nitrobenzamide (800 mg, 80% yield) as a white solid. LCMS (Method B): 0.56 min [MH]⁺=180.5.

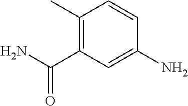

### Step 3: 5-amino-2-methylbenzamide

To a solution of 2-methyl-5-nitrobenzamide (320 mg, 1.78 mmol) in methanol (30 mL) were added zinc powder (1.5 g) and saturated ammonium chloride aqueous solution (30 mL). The resulting mixture was stirred at room temperature overnight. The solid was removed via filtration, and the filtrate was removed on a rotary evaporator to give a residue which was partitioned between water and ethylacetate. The organic phase was washed with water, brine, dried over sodium sulfate and concentrated to give 5-amino-2-methylbenzamide as a light yellow solid (250 mg, 94% yield). LCMS (Method B): 0.27 min [MH]⁺=151.1.

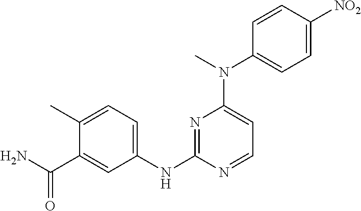 [SMILES: Cc1ccc(Nc2nccc(N(C)c3ccc([N+](=O)[O-])cc3)n2)cc1C(N)=O]

### Step 4: 2-methyl-5-((4-(methyl(4-nitrophenyl)amino)pyrimidin-2-yl)amino) benzamide

To a solution of 5-amino-2-methylbenzamide (250 mg, 1.66 mmol) in 1,4-dioxane (20 mL) were added 2-chloro-N-methyl-N-(4-nitrophenyl)pyrimidin-4-amine (439 mg, 1.66 mmol) and p-toluenesulfonic acid monohydrate (253 mg, 1.33 mmol). The mixture was stirred at 120° C. for 3 hours at reflux. The excess dioxane was removed on rotary evaporator to give a residue which was triturated with saturated ammonia aqueous solution (30 mL). The solid was collected via filtration, dried under reduced pressure to give 2-methyl-5-((4-(methyl(4-nitrophenyl)amino)pyrimidin-2-yl)amino)benzamide as a brown solid (590 mg, 94% yield). LCMS (Method B): 1.24 min [MH]⁺=379.1.

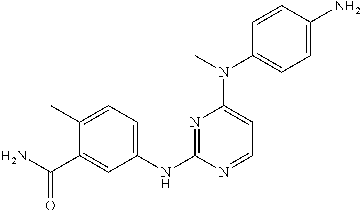 [SMILES: Cc1ccc(Nc2nccc(N(C)c3ccc(N)cc3)n2)cc1C(N)=O]

### Step 5: 5-((4-((4-aminophenyl)(methyl)amino)pyrimidin-2-yl)amino)-2-methyl-benzamide

To a solution of 5-(4-(N-methyl-N-(4-nitrophenyl)amino) pyrimidin-2-ylamino)-2-methyl benzamide (230 mg, 0.60 mmol) in methanol (20 mL) were added zinc powder (1.0 g) and saturated ammonium chloride aqueous solution (20 mL), and the mixture was stirred at room temperature overnight. The solid was removed via filtration and the filtrate was concentrated to give a residue which was partitioned between water and ethylacetate. The organic phase was washed with water, brine, dried over sodium sulfate and concentrated under reduced pressure to give 5-((4-((4-aminophenyl)(methyl)amino)pyrimidin-2-yl)amino)-2-methylbenzamide (180 mg, 86% yield) as a light yellow solid. LCMS (Method B): 0.49 min [MH]⁺=349.1.

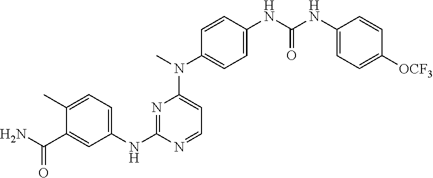 [SMILES: Cc1ccc(Nc2nccc(N(C)c3ccc(NC(=O)Nc4ccc(OC(F)(F)F)cc4)cc3)n2)cc1C(N)=O]

### Step 6: 2-Methyl-5-((4-(methyl(4-(3-(4-(trifluoromethoxy)phenyl)ureido) phenyl) amino)pyrimidin-2-yl)amino) benzamide

To a solution of 4-(N-(4-aminophenyl)-N-methylamino)-N-methylpyrimidine-2-carboxamide (180 mg, 0.52 mmol) in tetrahydrofuran (10 mL) were added 4-(trifluoromethoxy)phenyl isocyanate (106 mg, 0.52 mmol) and N,N-diisopropylethylamine (134 mg, 1.04 mmol). The mixture was stirred at room temperature for 3 hours. The reaction mixture was concentrated to dryness under reduced pressure to give a residue which was purified by silica-gel chromatography (dichloromethane/methanol, 25:1) to give 2-methyl-5-((4-(methyl(4-(3-(4-(trifluoromethoxy) phenyl)ureido)phenyl) amino)pyrimidin-2-yl)amino)benzamide (110 mg, 38%) as a light brown solid. ¹H NMR (400 MHz, DMSO-d₆): δ ppm 2.34 (s, 3H), 3.48 (s, 3H), 5.94 (br s, 1H), 7.54-7.13 (m, 7H), 7.88-7.54 (m, 7H), 9.38 (s, 2H), 10.47 (s, 1H). LCMS (Method B): 2.48 min [MH]⁺=552.2.

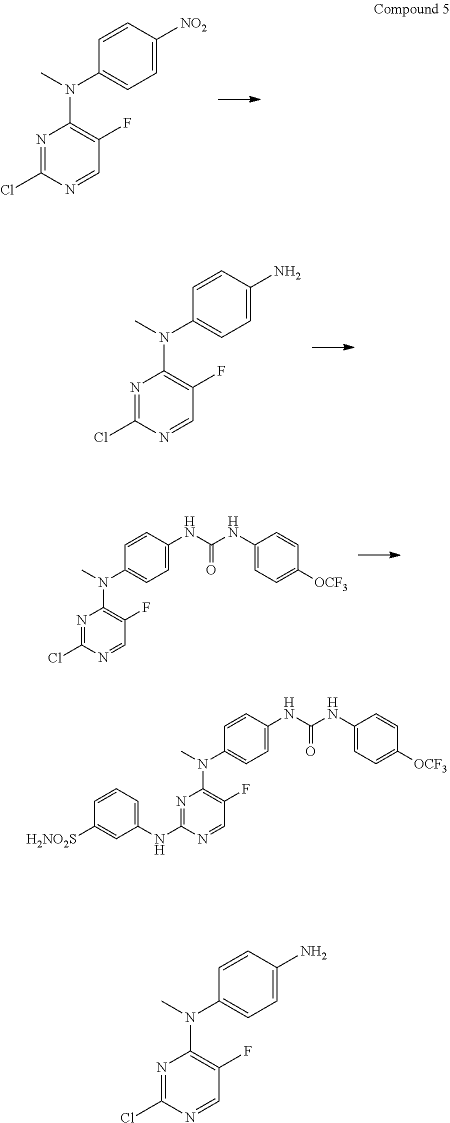 [SMILES: CN(c1ccc([N+](=O)[O-])cc1)c1nc(Cl)ncc1F ; CN(c1ccc(N)cc1)c1nc(Cl)ncc1F ; CN(c1ccc(NC(=O)Nc2ccc(OC(F)(F)F)cc2)cc1)c1nc(Cl)ncc1F ; CN(c1ccc(NC(=O)Nc2ccc(OC(F)(F)F)cc2)cc1)c1nc(Nc2cccc(S(N)(=O)=O)c2)ncc1F ; CN(c1ccc(N)cc1)c1nc(Cl)ncc1F]

### Step 1: N1-(2-chloro-5-fluoropyrimidin-4-yl)-N1-methylbenzene-1,4-diamine

To the mixture of 2-chloro-5-fluoro-N-methyl-N-(4-nitrophenyl)pyrimidin-4-amine (200 mg, 0.708 mmol) in methanol was added zinc powder (460 mg, 7.08 mmol) and ammonium chloride solution (3 mL). The resulting mixture was stirred at 60° C. for 3 hours. Zinc powder was filtered off and the filtrate was partitioned between ethylacetate and 1 M sodium hydroxide solution. The aqueous layer was extracted with ethylacetate several times. The combined organic layers were washed with brine, dried over sodium sulfate and concentrated to give N1-(2-chloro-5-fluoropyrimidin-4-yl)-N1-methylbenzene-1,4-diamine (172 mg, 96%) as a yellow solid. LCMS (Method B): 1.31 min [MH]⁺=253.1.

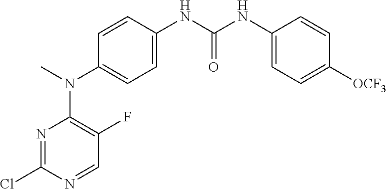 [SMILES: CN(c1ccc(NC(=O)Nc2ccc(OC(F)(F)F)cc2)cc1)c1nc(Cl)ncc1F]

### Step 2: 1-(4-((2-chloro-5-fluoropyridin-4-yl)(methyl)amino)phenyl)-3-(4-(trifluoro-methoxy)phenyl)urea

A solution of N1-(2-chloro-5-fluoropyrimidin-4-yl)-N1-methylbenzene-1,4-diamine (172 mg, 0.68 mmol) in dichloromethane (3 mL) was treated with 1-isocyanato-4-(trifluoromethoxy)benzene (138 mg, 0.68 mmol). The reaction mixture was stirred at room temperature for 4 hours. The precipitated solid was collected via filtration, rinsed with dichloromethane and dried under reduced pressure to give 1-(4-((2-chloro-5-fluoropyridin-4-yl)(methyl)amino)phenyl)-3-(4-(trifluoromethoxy)phenyl) urea (255 mg, 82%) as a white solid. LCMS (Method B): 3.11 min [MH]⁺=456.1

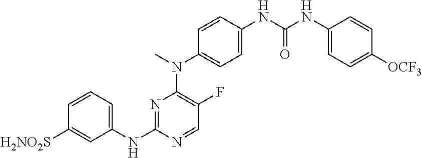 [SMILES: CN(c1ccc(NC(=O)Nc2ccc(OC(F)(F)F)cc2)cc1)c1nc(Nc2cccc(S(N)(=O)=O)c2)ncc1F]

### Step 3: 3-((5-Fluoro-4-(methyl(4-(3-(4-(trifluoromethoxy) phenyl)ureido) phenyl) amino)pyrimidin-2-yl) amino) benzenesulfonamide

To a flask were added 1-(4-((2-chloro-5-fluoropyridin-4-yl)(methyl)amino) phenyl)-3-(4-(trifluoromethoxy)phenyl)urea (100 mg, 0.22 mmol), 3-aminophenylsulfamide (38 mg, 0.22 mmol), TsOH.H₂O (85 mg, 0.44 mmol) and DMF (1.5 mL). The resulting mixture was stirred was stirred at 60° C. for 16 h. The resulting mixture was partitioned between ethyl acetate and potassium carbonate solution. The organic layer was washed with brine and dried over sodium sulfate. After purified by preparative HPLC, 3-((5-fluoro-4-(methyl(4-(3-(4-(trifluoromethoxy)phenyl)ureido)phenyl)amino)pyrimidin-2-yl)amino)-benzenesulfonamide was obtaind (10 mg, 8%) as a white solid. ¹H NMR (400 MHz, MeOD-d₄): δ ppm 3.64 (s, 3H), 7.24 (d, J=8.8 Hz, 2H), 7.34 (d, J=8.4 Hz, 2H), 7.52 (m, 5H), 7.64 (m, 1H), 7.71 (d, J=7.6 Hz, 1H), 7.96 (d, J=6.8 Hz, 1H), 8.47 (s, 1H). LCMS (Method B): 2.83 min [MH]⁺=592.2, [MNa]⁺=614.1.

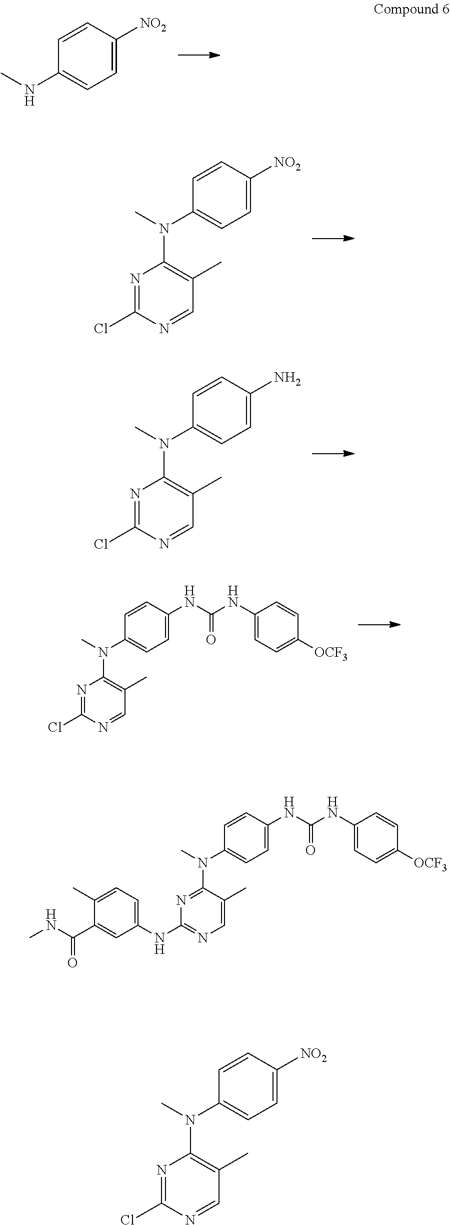 [SMILES: CNc1ccc([N+](=O)[O-])cc1 ; Cc1cnc(Cl)nc1N(C)c1ccc([N+](=O)[O-])cc1 ; Cc1cnc(Cl)nc1N(C)c1ccc(N)cc1 ; Cc1cnc(Cl)nc1N(C)c1ccc(NC(=O)Nc2ccc(OC(F)(F)F)cc2)cc1 ; CNC(=O)c1cc(Nc2ncc(C)c(N(C)c3ccc(NC(=O)Nc4ccc(OC(F)(F)F)cc4)cc3)n2)ccc1C ; Cc1cnc(Cl)nc1N(C)c1ccc([N+](=O)[O-])cc1]

### Step 1: 2-chloro-N,5-dimethyl-N-(4-nitrophenyl)pyrimidin-4-amine

A solution of N-methyl-4-nitroaniline (3 g, 19.7 m mol) in DMSO (20 mL) was added 2,4-dichloro-5-methylpyrimidine (6.4 g, 39.4 mmol) and Cs₂CO₃ (12.8 g, 39.4 mmol). The resulting mixture was heated to 100° C. under nitrogen for 16 hours. The organic layer was partitioned between water ans EtOAc. The organics were separated, dried over sodium sulfate and concentrated under reduced pressure to give a residue which was purified by column chromatography on silica gel (PE/EtOAC=25/1) to give the titled product (1.0 g, 18%) as a yellow solid. LCMS (acidic 5 min): 3.03 min [MH]⁺=279.0

The 2-addition product 4-chloro-N,5-dimethyl-N-(4-nitrophenyl)pyrimidin-2-amine was also isolated (1.1 g, 20%) as a yellow solid LCMS (acidic 5 min): 2.60 min [MH]⁺=279.0.

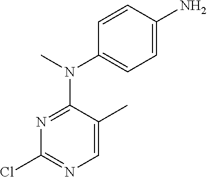 [SMILES: Cc1cnc(Cl)nc1N(C)c1ccc(N)cc1]

Step 2: N1-(2-chloro-5-methylpyrimidin-4-yl)-N1-methylbenzene-1,4-diamine

To a solution of 2-chloro-N,5-dimethyl-N-(4-nitrophenyl)pyrimidin-4-amine (1 g, 3.6 mmol) in MeOH (10 mL) was added zinc (1.4 g, 22 mmol) and aqueous NH₄Cl solution (10 mL). The reaction mixture was stirred at 60° C. under nitrogen for 16 hours. The reaction mixture was then concentrated under reduced pressure and the residue was diluted with ethyl acetate (10 mL), and washed with H₂O (10 mL). The organic layer was separated, dried over sodium sulfate and concentrated under reduced pressure. The residue was purified by column chromatography on silica gel (petroleum ether/ethyl acetate=20/1) to give the titled product (600 mg, 67%) as a yellow solid. LCMS (acidic 5 min): 1.74 min [MH]⁺=249.1.

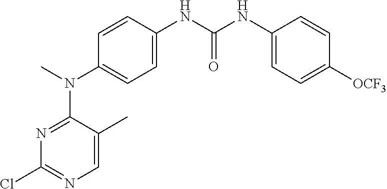 [SMILES: Cc1cnc(Cl)nc1N(C)c1ccc(NC(=O)Nc2ccc(OC(F)(F)F)cc2)cc1]

### Step 3: 1-(4-((2-chloro-5-methylpyrimidin-4-yl)(methyl)amino)phenyl)-3-(4-(trifluoromethoxy)phenyl)urea

To a solution of N1-(2-chloro-5-methylpyrimidin-4-yl)-N1-methylbenzene-1,4-diamine (500 mg, 2 mmol) in DCM (10 mL) was added 1-isocyanato-4-(trifluoromethoxy)benzene (812 mg, 4 mmol). The resulting mixture was stirred at room temperature under nitrogen for 16 hours. The solid was collected by filtration, washed with diethylether and dried under reduced pressure to give the titled product (500 mg, 55%) as a yellow solid. LCMS (acidic 5 min): 3.25 min [MH]⁺=452.1.

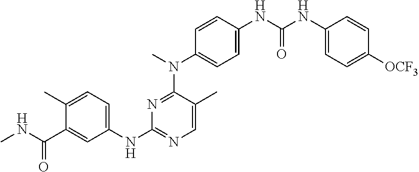 [SMILES: CNC(=O)c1cc(Nc2ncc(C)c(N(C)c3ccc(NC(=O)Nc4ccc(OC(F)(F)F)cc4)cc3)n2)ccc1C]

### Step 4: N,2-dimethyl-5-((5-methyl-4-(methyl(4-(3-(4-(trifluoromethoxy)phenyl) ureido)phenyl)amino)pyrimidin-2-yl)amino)benzamide

To a solution of 1-(4-((2-chloro-5-methylpyrimidin-4-yl)(methyl)amino) phenyl)-3-(4-(trifluoromethoxy)phenyl)urea (200 mg, 0.44 mmol) in iso-propanol (10 mL) was added 5-amino-N,2-dimethylbenzamide (73 mg, 0.44 mmol) and HCl (2 drops). The resulting mixture was heated to 90° C. under nitrogen for 16 hours. The solid was collected by filtration, washed with ether and dried to give the desired product (150 mg, 58%) as a yellow solid.

LCMS (acidic 5 min): 2.57 min [MH]⁺=580.3. ¹H NMR (400 MHz, DMSO-d₆) δ ppm 1.39 (s, 3 H), 2.24 (s, 3H), 2.73 (d, J=4.4 Hz, 3H), 3.37 (s, 3H), 7.10-7.12 (m, 3H), 7.14-7.27 (m, 2H), 7.29 (d, J=8.8 Hz, 2H), 7.49 (d, J=8.8 Hz, 2H), 7.63 (dd, J=8.0, 2.0 Hz, 1H), 7.79 (s, 1H), 7.88 (d, J=1.6 Hz, 1H), 8.11 (br s,1H), 9.06 (s, 1H), 9.14 (s, 1H), 9.35 (s, 1H).

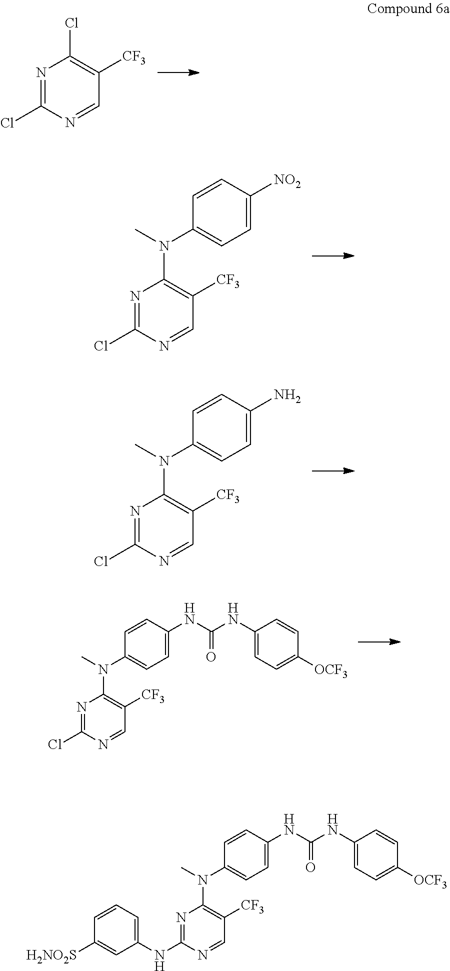 [SMILES: FC(F)(F)c1cnc(Cl)nc1Cl ; CN(c1ccc([N+](=O)[O-])cc1)c1nc(Cl)ncc1C(F)(F)F ; CN(c1ccc(N)cc1)c1nc(Cl)ncc1C(F)(F)F ; CN(c1ccc(NC(=O)Nc2ccc(OC(F)(F)F)cc2)cc1)c1nc(Cl)ncc1C(F)(F)F ; CN(c1ccc(NC(=O)Nc2ccc(OC(F)(F)F)cc2)cc1)c1nc(Nc2cccc(S(N)(=O)=O)c2)ncc1C(F)(F)F]

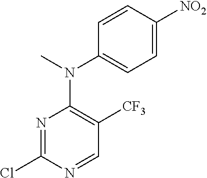 [SMILES: CN(c1ccc([N+](=O)[O-])cc1)c1nc(Cl)ncc1C(F)(F)F]

### Step 1: 2-chloro-N-methyl-N-(4-nitrophenyl)-5-(trifluoromethyl)pyrimidin-4-amine

To a flask were added N-methyl-4-nitroaniline (175 mg, 1.15 mmol), 2-chloro-N-methyl-N-(4-nitrophenyl)-5-(trifluoromethyl)pyrimidin-4-amine (250 mg, 1.15 mmol), DIEA (31.8 mg, 2.3 mmol) and dry THF (10 mL). The resulting mixture was stirred at 90° C. for 20 hours. The solvent was removed and the crude product was purified by column chromatography (petroleum ether/ethylacetate, 15:1) to give 2-chloro-N-methyl-N-(4-nitrophenyl)-5-(trifluoromethyl)pyrimidin-4-amine (300 mg, 78%) as a yellow solid. LCMS (method B): 3.10 min [MH]+=333.0.

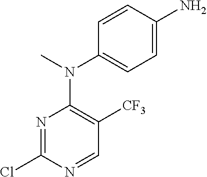 [SMILES: CN(c1ccc(N)cc1)c1nc(Cl)ncc1C(F)(F)F]

### Step 2: N1-(2-chloro-5-(trifluoromethyl)pyrimidin-4-yl)-N1-methylbenzene-1,4-diamine

To the mixture of 2-chloro-N-methyl-N-(4-nitrophenyl)-5-(trifluoro-methyl)pyrimidin-4-amine (150 mg, 0.45 mmol) in methanol (6 mL) were added zinc podwer (294 mg, 4.5 mmol) and saturated NH4Cl solution (3 mL). The resultant mixture was stirred at 60° C. for 3 hours. Zinc powder was filtered off and the filtrate was partitioned between ethylacetate and sodium hydroxide solution (1 M). The organic layer was separated and the aqueous layer was extracted with ethylacetate. The combined organic layers were washed with brine, dried and concentrated to give N1-(2-chloro-5-(trifluoromethyl)pyrimidin-4-yl)-N1-methylbenzene-1,4-diamine (128 mg, 94%) as a yellow solid. LCMS (method B): 2.78 min [MH]+=303.1.

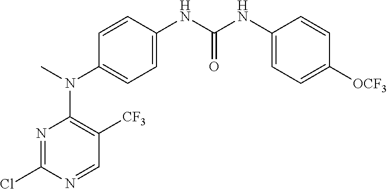 [SMILES: CN(c1ccc(NC(=O)Nc2ccc(OC(F)(F)F)cc2)cc1)c1nc(Cl)ncc1C(F)(F)F]

### Step 3: 1-(4-((2-chloro-5-(trifluoromethyl)pyrimidin-4-yl)(methyl)amino)phenyl)-3-(4-(trifluoromethoxy) phenyl)urea

To a solution of N1-(2-chloro-5-(trifluoromethyl)pyrimidin-4-yl)-N1-methylbenzene-1,4-diamine (128 mg, 0.423 mmol) in dichloromethane (5 mL) was added 1-isocyanato-4-(trifluoromethoxy)benzene (86 mg, 0.423 mmol). The resulting mixture was stirred at room temperature overnight. The precipitate that formed was collected via filtration, washed with dichloromethane and dried to give 1-(4-((2-chloro-5-(trifluoromethyl)pyrimidin-4-yl)(methyl)amino)phenyl)-3-(4-(trifluoromethoxy) phenyl)urea (150 mg, 70%) as a white solid. LCMS (method B): 3.51 min [MH]+=506.1.

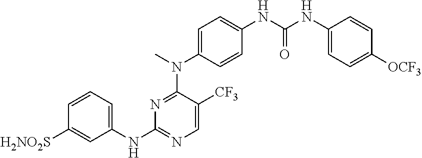 [SMILES: CN(c1ccc(NC(=O)Nc2ccc(OC(F)(F)F)cc2)cc1)c1nc(Nc2cccc(S(N)(=O)=O)c2)ncc1C(F)(F)F]

### Step 4: 3-((4-(Methyl(4-(3-(4-(trifluoromethoxy)phenyl)ureido)phenyl)amino)-5-(trifluoromethyl) pyrimidin-2-yl)amino)benzenesulfonamide

To a flask were added 1-(4-((2-chloro-5-trifluoromethylpyridin-4-yl)(methyl)amino)phenyl)-3-(4-(trifluoromethoxy)phenyl)urea (70 mg, 0.138 mmol), 3-aminophenylsulfamide (24 mg, 0.138 mmol), TsOH.H2O (53 mg, 0.277 mmol) and DMF (2 mL). The resulting mixture was stirred at 60° C. overnight. The resulting mixture was partitioned between ethylacetate and potassium carbonate solution. The organic layer was washed with brine and dried over sodium sulfate and purified by preparative TLC to afford 3-((4-(methyl(4-(3-(4-(trifluoromethoxy)phenyl)ureido)phenyl)amino)-5-(trifluoro-methyl)pyrimidin-2-yl)amino)benzenesulfonamide (42 mg, 49%) as a white solid. 1H NMR (400 MHz, DMSO-d6): δ ppm 3.38 (s, 3H), 7.23 (d, J=8.8 Hz, 2H), 7.32 (m, 5H), 7.49 (m, 3H), 7.60 (m, 2H), 7.76 (d, J=7.6 Hz, 1H), 8.09 (s, 1H), 8.32 (s, 1H), 8.83 (d, J=8.0 Hz, 2H), 8.93 (s, 1H). LCMS (method B): 3.02 min [MH]+=642.2, [MNa]+=664.1.

Compound 7

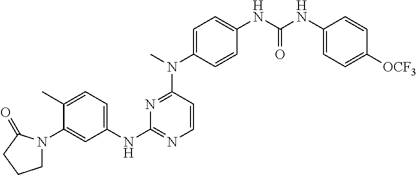 [SMILES: Cc1ccc(Nc2nccc(N(C)c3ccc(NC(=O)Nc4ccc(OC(F)(F)F)cc4)cc3)n2)cc1N1CCCC1=O]

Following general procedure C using intermediate C (182 mg, 0.415 mmol) and 1-(5-amino-2-methylphenyl)pyrrolidin-2-one (from step 3 intermediate H, 79 mg, 0.415 mmol), 2-Methyl-5-((4-(methyl(4-(3-(4-(trifluoromethoxy)phenyl) ureido)phenyl)amino)pyrimidin-2-yl)amino)benzenesulfonamide (90 mg, 37%) was obtained as a white solid.

¹H NMR (400 MHz, DMSO-d₆): δ ppm 2.06 (s, 3H), 2.14 (m, 2H), 2.43 (m, 2H), 3.40 (s, 3H), 3.65 (t, J=6.8 Hz, 2H), 5.77 (d, J=6 Hz, 1H), 7.12 (d, J=8.4 Hz, 1H), 7.31 (m, 4H), 7.59 (m, 5H), 7.70 (s, 1H), 7.86 (d, J=6 Hz, 1H), 8.89 (s, 1H), 8.94 (s, 1H), 9.15 (s, 1 H). LCMS (method B): 2.64 min [MH]⁺=592.3 [MNa]⁺=614.2.

Compound 8

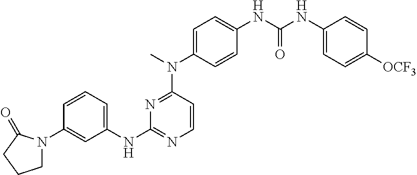 [SMILES: CN(c1ccc(NC(=O)Nc2ccc(OC(F)(F)F)cc2)cc1)c1ccnc(Nc2cccc(N3CCCC3=O)c2)n1]

1-(4-(Methyl(2-((3-(2-oxopyrrolidin-1-yl)phenyl)amino)pyrimidin-4-yl)amino)phenyl)-3-(4-(trifluoromethoxy)phenyl)urea.

Following general procedure C using intermediate C (100 mg, 0.23 mmol) and 1-(3-aminophenyl)pyrrolidin-2-one (40 mg, 0.23 mmol), 1-(4-(Methyl(2-((3-(2-oxopyrrolidin-1-yl)phenyl)amino)pyrimidin-4-yl)amino)phenyl)-3-(4-(trifluoro-methoxy)phenyl)urea (80 mg, 61%) was obtained as a white solid. ¹H NMR (400 MHz, DMSO-d₆): δ ppm 2.09 (m, 2H), 2.48 (t, J=6.8 Hz, 2H), 3.42 (s, 3H), J=7.2 Hz, 2H), 5.79 (d, J=6.0 Hz, 1H), 7.21 (t, J=8.4 Hz, 1H), 7.32 (m, 5H), 7.59 (m, 5H), 7.88 (d, J=6.0 Hz, 1H), 8.01 (s, 1H), 8.87 (s, 1H), 8.92 (s, 1H), 9.16 (s, 1H). LCMS (Method B): 2.61 min [MH]⁺=578.2, [MNa]⁺=600.2.

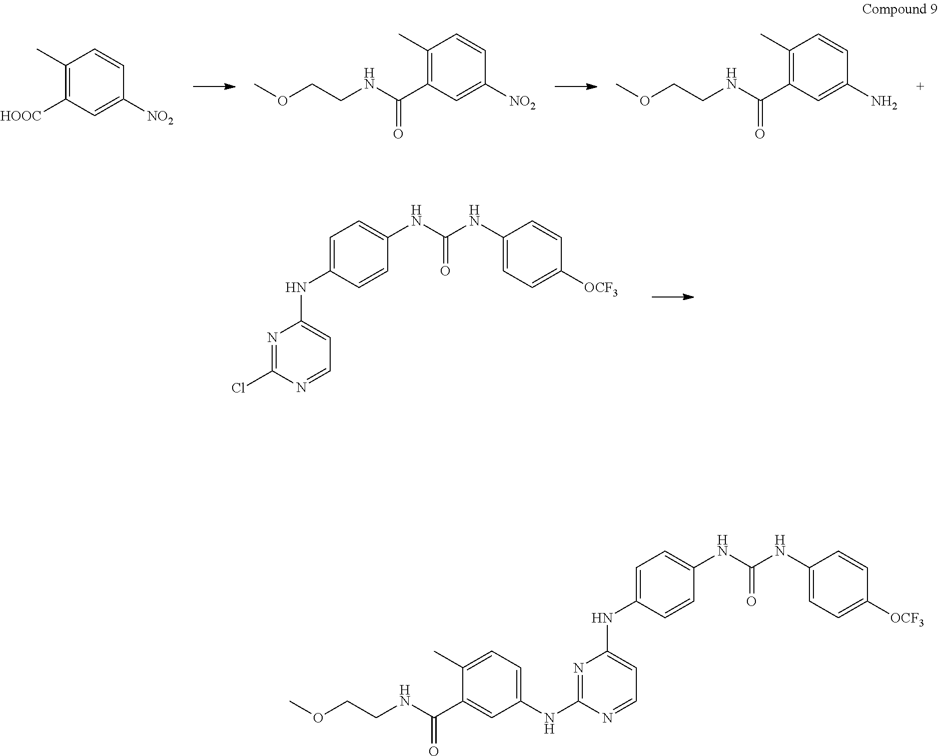 [SMILES: Cc1ccc([N+](=O)[O-])cc1C(=O)O ; COCCNC(=O)c1cc([N+](=O)[O-])ccc1C ; COCCNC(=O)c1cc(N)ccc1C ; "+" ; O=C(Nc1ccc(Nc2ccnc(Cl)n2)cc1)Nc1ccc(OC(F)(F)F)cc1 ; COCCNC(=O)c1cc(Nc2nccc(Nc3ccc(NC(=O)Nc4ccc(OC(F)(F)F)cc4)cc3)n2)ccc1C]

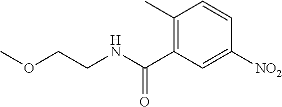 [SMILES: COCCNC(=O)c1cc([N+](=O)[O-])ccc1C]

### Step 1: N-(2-methoxyethyl)-2-methyl-5-nitrobenzamide

To solution of 2-methyl-5-nitrobenzoic acid (500 mg, 2.76 mmol) in DMF (30 mL) was added HATU (1.26 g, 3.31 mmol), TEA (838 mg, 8.28 mmol) and 2-methoxyethanamine (228 mg, 3.04 mmol). The solution was stirred at RT overnight under N₂. The mixture was diluted with ethyl acetate (150 mL) and water (70 mL). The organic layer was separated and aqueous layer was extracted with ethylacetate. The combined organic phase was washed with water, brine, dried over sodium sulfate and concentrated to give a residue which was purified by column chromatography on silica gel (DCM/MeOH, 150:1 to 100:1) to give N-(2-methoxyethyl)-2-methyl-5-nitrobenzamide (560 mg, 85%) as pale-yellow solid. LCMS (Method B): 0.93min [MH]⁺=239.1, [MNa]⁺=261.1, [2MNa]⁺=499.2.

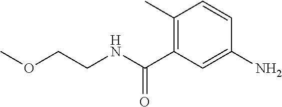

### Step 2: 5-amino-N-(2-methoxyethyl)-2-methylbenzamide

To a solution of N-(2-methoxyethyl)-2-methyl-5-nitrobenzamide (130 mg, 0.54 mmol) in EtOAC (12 mL) was added 10% Pd/C (30 mg). The reaction mixture was stirred under a hydrogen atmosphere overnight at RT. The catalyst was removed by filtration and the solvent was removed under reduced pressure to give a residue which was purified by column chromatography on silica gel (DCM/MeOH, 50:1) to give 5-amino-N-(2-methoxyethyl)-2-methylbenzamide (89 mg, 78%) as pale-red solid. LCMS (Method B): 0.29 min [MH]⁺=209.1, [2MNa]⁺=439.2.

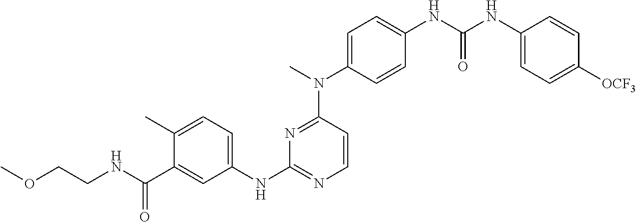 [SMILES: COCCNC(=O)c1cc(Nc2nccc(N(C)c3ccc(NC(=O)Nc4ccc(OC(F)(F)F)cc4)cc3)n2)ccc1C]

### Step 3: N-(2-Methoxyethyl)-2-methyl-5-((4-(methyl(4-(3-(4-(trifluoromethoxy) phenyl)ureido)phenyl)amino)pyrimidin-2-yl)amino)benzamide

To a solution of intermediate C (183 mg, 0.42 mmol), 5-amino-N-(2-methoxyethyl)-2-methylbenzamide (87 mg, 0.42 mmol) in isopropanol (5 mL) was added concentrated HCl (3 drops). The resulting mixture was stirred at 85° C. overnight under N₂. The mixture was allowed to cool down to room temperature. Saturated sodium carbonate solution was added to newtralize the mixture, followed by addition of the water and ethylacetate. The organic layer was separated and aqueous layer was extracted with ethylacetate several times. The combined organic layers were washed with water, brine, dried over sodium sulfate and concentrated to give a residue which was purified by column chromatography (DCM/MeOH, 80:1 to 20:1) to give N-(2-methoxyethyl)-2-methyl-5-((4-(methyl(4-(3-(4-(trifluoromethoxy)phenyl)ureido) phenyl)amino)pyrimidin-2-yl)amino)benzamide (108 mg, 42%) as a white solid. ¹H NMR (400 MHz, DMSO-d₆): δ ppm 2.24 (s, 3H), 3.27 (s, 3H), 3.45 (m, 7H), 5.76 (d, J=6 Hz, 1H), 7.08 (d, J=8.4 Hz, 1H), 7.31 (m, 4H), 7.65 (m, 5H), 7.86 (d, J=6 Hz, 2H), 8.18 (t, J=5.6 Hz, 1H), 8.89 (s, 1H), 8.95 (s, 1H), 9.18 (s, 1H). LCMS (Method B): 2.61 min [MH]⁺=610.3.

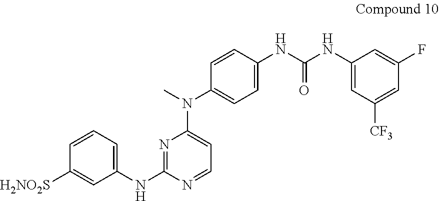 [SMILES: CN(c1ccc(NC(=O)Nc2cc(F)cc(C(F)(F)F)c2)cc1)c1ccnc(Nc2cccc(S(N)(=O)=O)c2)n1]

3-((4-((4-(3-(3-fluoro-5-(trifluoromethyl)phenyl)ureido)phenyl)(methyl)amino) pyrimidin-2-yl)amino)benzenesulfonamide

Following general procedure B using intermediate B (100 mg, 0.2 mmol) and 3-fluoro-5-(trifluoromethyl)aniline (37 mg, 0.2 mmol), 3-((4-((4-(3-(3-fluoro-5-(trifluoromethyl)phenyl)ureido)phenyl)(methyl)amino)pyrimidin-2-yl)amino) benzenesulfonamide (12 mg, 10%) was obtained as a white solid.

¹H NMR (400 MHz, DMSO-d₆): δ ppm 3.47 (s, 3H), 5.86 (d, J=6.4 Hz, 1H), 7.32 (m, 5H), 7.47 (m, 2H), 7.66 (m, 3H), 7.72 (s, 1H), 7.80 (m, 1H), 7.90 (d, J=6.4 Hz, 1H), 8.49 (s, 1H), 9.29 (m, 1H), 9.55 (m, 1H), 9.85 (m, 1H). LCMS (Method B): 2.60 min [MH]⁺=576.2, [MNa]⁺=598.2.

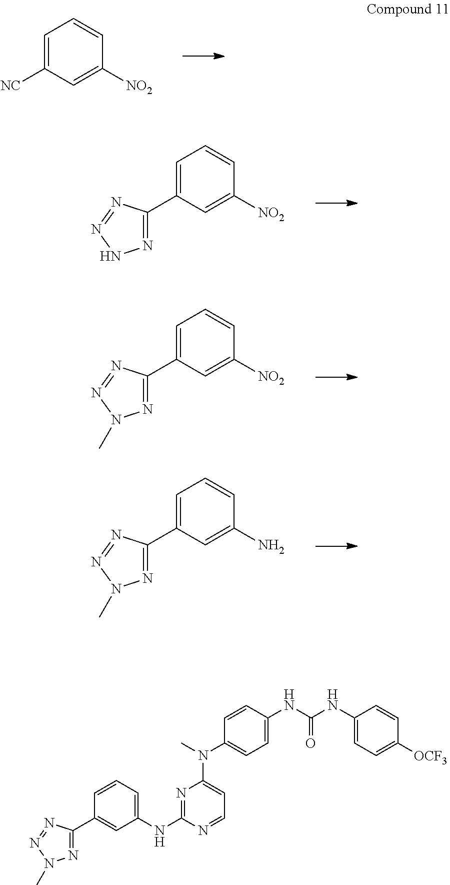 [SMILES: N#Cc1cccc([N+](=O)[O-])c1 ; O=[N+]([O-])c1cccc(-c2nn[nH]n2)c1 ; Cn1nnc(-c2cccc([N+](=O)[O-])c2)n1 ; Cn1nnc(-c2cccc(N)c2)n1 ; CN(c1ccc(NC(=O)Nc2ccc(OC(F)(F)F)cc2)cc1)c1ccnc(Nc2cccc(-c3nnn(C)n3)c2)n1]

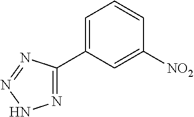 [SMILES: O=[N+]([O-])c1cccc(-c2nn[nH]n2)c1]

### Step 1: 5-(3-nitrophenyl)-2H-tetrazole

To a solution of anhydrous AlCl₃ (0.27 g, 2.03 mmol) in anhydrous NMP (5 mL) were added NaN₃ (2.63 g, 40 mmol) and 3-nitrobenzonitrile (2 g, 13.5 mmol). The mixture was stirred for 1 min and was subsequently irradiated in a microwave instrument at 200° C. for 5 min. The reaction mixture was poured into ice water. The pH of the solution was adJusted to 1 with concentrated HCl. The solid formed was collected by filtration and washed thoroughly with cold 1N HCl to give 5-(3-nitrophenyl)-2H-tetrazole (3.30 g, 100%) as a yellow solid. LCMS (Method B): 1.07 min [MH]⁺=192.1, [MNa]⁺=214.0.

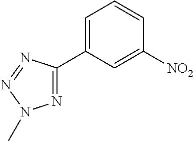

### Step 2: 3-(2-methyl-2H-tetrazol-5-yl)benzenamine

To a solution of 5-(3-nitrophenyI)-1H-tetrazole (1.0 g, 5.23 mmol) in THF (30 mL) was added NaH (251 mg, 10.5 mmol) at 0° C. The mixture was stirred for 15 min and then CH₃I (817.5 mg, 5.76 mmol) was added. The reaction mixture was stirred at room temperature for 3 hours. The reaction mixture was poured into water and extracted with ethylacetate. The organic phase was washed with brine, dried and concentrated to give a residue. The crude product was purified by column chromatography on silica gel (petroleum ether/ethylacetate, 10:1) to give 3-(2-methyl-2H-tetrazol-5-yl)benzenamine (160 mg, 15%) as a yellow solid. LCMS (Method B): 2.20 min [MH]⁺=206.1, [MNa]⁺=228.0.

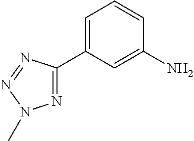

### Step 3: 3-(2-methyl-2H-tetrazol-5-yl)benzenamine

To a solution of 3-(2-methyl-2H-tetrazol-5-yl)benzenamine (105 mg, 0.51 mmol) in ethylacetate (2 mL) was added Pd/C (10%, 20 mg). The mixture was stirred under an hydrogen atmosphere at room temperature overnight. The Pd/C was filtered off by filtration and the filtrate was concentrated to give 3-(2-methyl-2H-tetrazol-5-yl)benzenamine (73 mg, 81%) as a yellow oil. LCMS (Method B): 0.40 min [MH]⁺=176.1.

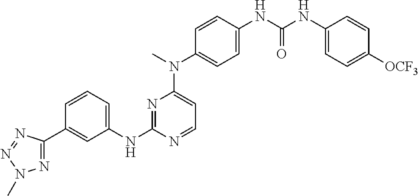 [SMILES: CN(c1ccc(NC(=O)Nc2ccc(OC(F)(F)F)cc2)cc1)c1ccnc(Nc2cccc(-c3nnn(C)n3)c2)n1]

### Step 4: 1-(4-(Methyl(2-((3-(2-methyl-2H-tetrazol-5-yl)phenyl)amino)pyrimidin-4-yl)amino)phenyl)-3-(4-(trifluoromethoxy)phenyl)urea

To a solution of 3-(2-methyl-2H-tetrazol-5-yl)benzenamine (50 mg, 0.29 mmol) in isopropanol (5 mL) were added intermediate C (125 mg, 0.29 mmol) and concentrated HCl (2 drops). The mixture was stirred at 85° C. overnight and poured into 1 M NaOH solution. The resulting mixture was extracted with ethylacetate. The organic phase was washed with brine, dried and concentrated to give a residue. The crude product was purified by column chromatography on silica gel (DCM/MeOH, 40:1) to give 1-(4-(methyl(2-((3-(2-methyl-2H-tetrazol-5-yl)phenyl) amino)pyrimidin-4-yl)amino)phenyl)-3-(4-(trifluoromethoxy)phenyl)urea (41.4 mg, 25%) as a yellow solid. ¹H NMR (400 MHz, DMSO-d₆): δ ppm 3.49 (s, 3H), 4.42 (s, 3H), 5.81 (d, J=6.0 Hz, 1H), 7.32 (m, 4H), 7.41 (t, J=8.0 Hz, 1H), 7.60 (m, 5H), 7.81 (d, J=8.4 Hz, 1H), 7.91 (d, J=5.6 Hz, 1H), 8.81 (s, 1H), 8.88 (s, 1H), 8.93 (s, 1H), 9.44 (s, 1H). LCMS (Method B): 2.72 min [MH]⁺=577.2, [MNa]⁺=599.2.

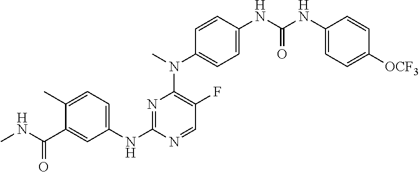 [SMILES: CNC(=O)c1cc(Nc2ncc(F)c(N(C)c3ccc(NC(=O)Nc4ccc(OC(F)(F)F)cc4)cc3)n2)ccc1C]

### 5-((5-Fluoro-4-(methyl(4-(3-(4-(trifluoromethoxy)phenyl)ureido)phenyl)amino) pyrimidin-2-yl) amino)-N,2-dimethylbenzamide

To a solution of 1-(4-((2-chloro-5-fluoropyrimidin-4-yl)(methyl)amino) phenyl)-3-(4-(trifluoromethoxy)phenyl)urea (from step 3 of the preparation of (5), 120 mg, 0.26 mmol) in i-PrOH (5 mL) was added 5-amino-N,2-dimethylbenzamide (step 3 of the preparation intermediate D, 43 mg, 0.26 mmol) and concentrated HCl (2 drops). The reaction mixture was stirred at 85° C. overnight under N₂. The mixture was diluted with ethylacetate (40 mL), NaOH (1 M, 15 mL) and water (15 mL). The aqueous layer was extracted again with ethylacetate. The combined organic phases were washed with water, brine, dried over sodium sulfate and concentrated to give a residue which was purified by column chromatography on silica gel (DCM/MeOH=100:1 to 40:1) to give 5-((5-fluoro-4-(methyl(4-(3-(4-(trifluoromethoxy)phenyl)ureido)phenyl) am ino)pyrimid in-2-yl)amino)-N,2-dimethylbenzamide (75 mg, 49%) as a pale-yellow solid. ¹H NMR (400 MHz, DMSO-d₆): δ ppm 2.23 (s, 3H), 2.74 (d, J=4.8 Hz, 3H), 3.43(s, 3H) 7.09 (d J=8.8 Hz, 1H), 7.24 (d J=8.8 Hz, 2H), 7.31 (d, J=8.4 Hz, 2H), 7.49 (d, J=8.8 Hz, 2H) 7.61 (m, 3H), 7.81 (d, J=2.4 Hz, 1H), 7.96 (d, J=5.6 Hz, 1H). 8.09 (m, 1H), 8.81 (s, 1H), 8.91 (s, 1H), 9.27 (s, 1H). LCMS (Method B): 2.85 min [MH]⁺=584.3, [MNa]⁺=606.2.

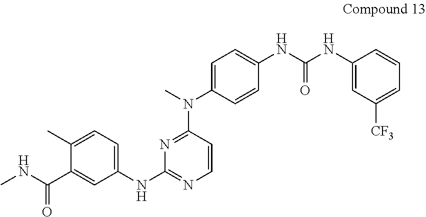 [SMILES: CNC(=O)c1cc(Nc2nccc(N(C)c3ccc(NC(=O)Nc4cccc(C(F)(F)F)c4)cc3)n2)ccc1C]

### N,N,2-trimethyl-5-((4-(methyl(4-(3-(3-(trifluoromethyl)phenyl)ureido)phenyl) amino)pyrimidin-2-yl)amino)benzamide

A solution of intermediate E (200 mg, 0.41 mmol) in THF (5 mL) was treated with 3-(trifluoromethyl)benzenamine (64 mg, 0.41 mmol) and DIEA (159 mg, 1.23 mmol). The reaction mixture was heated to 85° C. overnight. The solvent was removed and the crude was purified by column chromatography (DCM/methanol, 100:0 to 100:2) to give N,N,2-trimethyl-5-((4-(methyl(4-(3-(3-(trifluoromethyl)phenyl)ureido)phenyl)amino)pyrimidin-2-yl)amino)benzamide (55.3 mg, 30%) as a white solid. ¹H NMR (400 MHz, DMSO-d₆): δ ppm 2.23 (s, 3H), 2.74 (d, J=4.4 Hz, 3H), 3.41 (s, 3H), 5.77 (d, J=6.0 Hz, 1H), 7.08 (d, J=8.4 Hz, 1H), 7.28 (d, J=8.8 Hz, 2H), 7.34 (d, J=7.6 Hz, 1H), 7.66 (m, 5H), 7.87 (m, 2H), 8.05 (s, 1H), 8.15 (m, 1H), 8.96 (s, 1H), 9.12 (s, 1H), 9.18 (s, 1H). LCMS (Method B): 2.48 min [MH]⁺=550.2.

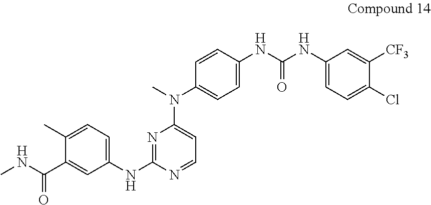 [SMILES: CNC(=O)c1cc(Nc2nccc(N(C)c3ccc(NC(=O)Nc4ccc(Cl)c(C(F)(F)F)c4)cc3)n2)ccc1C]

### 5-((4-((4-(3-(4-Chloro-3-(trifluoromethyl)phenyl)ureido)phenyl)(methyl)amino) pyrimidin-2-yl)amino)-N,2-dimethylbenzamide

To a solution of intermediate D (200 mg, 0.55 mmol) in THF (20 mL) were added 1-chloro-2-(trifluoromethyl)-4-isocyanatobenzene (128 mg, 0.58 mmol) and DIEA (142 mg, 1.1 mmol). The reaction mixture was stirred at room temperature for 16 hours, concentrated under reduced pressure and the crude solid was purified by column chromatography (DCM/methanol, 100:0 to 100:2) to give 5-((4-((4-(3-(4-chloro-3-(trifluoromethyl)phenyl)ureido)phenyl)(methyl) amino)pyrimidin-2-yl)amino)-N,2-dimethylbenzamide (86 mg, 27%) as a white solid. ¹H NMR (400 MHz, DMSO-d₆): δ ppm 2.23 (s, 3H), 2.74 (d, J=4.4 Hz, 3H), 3.41 (s, 3H), 5.77 (d, J=6.0 Hz, 1H), 7.08 (d, J=8.4 Hz, 1H), 7.28 (d, J=8.8 Hz, 2H), 7.66 (m, 5H), 7.87 (m, 2H), 8.10 (m, 1H), 8.15 (d, J=2.0 Hz, 1H), 9.01 (s, 1H), 9.18 (s, 1H), 9.23 (s, 1H). LCMS (Method B): 2.65 min [MH]⁺=584.2.

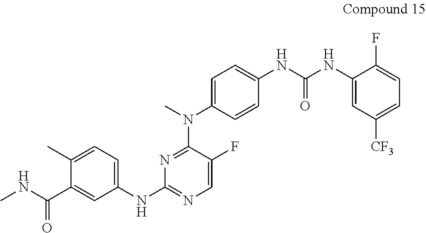 [SMILES: CNC(=O)c1cc(Nc2ncc(F)c(N(C)c3ccc(NC(=O)Nc4cc(C(F)(F)F)ccc4F)cc3)n2)ccc1C]

5-(5-fluoro-4-((4-(3-(2-fluoro-5-(trifluoromethyl)phenyl)ureido)phenyl)(methyl) amino)pyrimidin-2-ylamino)-N,2-dimethylbenzamide

To a solution of intermediate G (200 mg, 0.40 mmol) in THF (15 mL) was added 2-fluoro-5-(trifluoromethyl)aniline (72 mg, 0.40 mmol) and DIEA (155 mg, 1.20 mmol). The reaction mixture was heated to 85° C. overnight. The solvent was removed and the crude product was purified by column chromatography (DCM:methanol, 100-0 to 100:3) to give a white solid which was further purified by preparative HPLC to give 5-(5-fluoro-4-((4-(3-(2-fluoro-5-(trifluoromethyl)phenyl)ureido) phenyl) (methyl)amino)pyrimidin-2-ylamino)-N,2-dimethylbenzamide (20 mg, 9%) as a white solid. ¹H NMR (400 MHz, DMSO-d₆): δ ppm 2.23 (s, 3H), 2.74 (d, J=4.4 Hz, 3H), 3.44 (s, 3H), 7.10 (d, J=8.4 Hz, 1H), 7.27 (d, J=8.8 Hz, 2H), 7.41 (m, 1H), 7.48 (m, 3H), 7.58 (m, 1H), 7.80 (d, J=2.4 Hz, 1H), 7.98 (d, J=5.6 Hz, 1H), 8.11 (m, 1H), 8.64 (m, 1H), 8.93 (d, J=2.8 Hz, 1H), 9.27 (s, 1H), 9.32 (s, 1H). LCMS (method B): 2.88 min [MH]⁺=586.3.

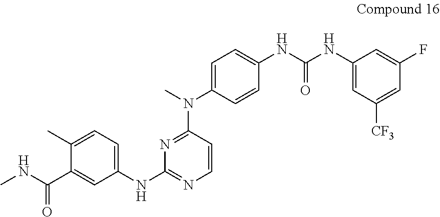 [SMILES: CNC(=O)c1cc(Nc2nccc(N(C)c3ccc(NC(=O)Nc4cc(F)cc(C(F)(F)F)c4)cc3)n2)ccc1C]

5-(4-((4-(3-(3-fluoro-5-(trifluoromethyl)phenyl)ureido)phenyl)(methyl)amino)-pyrimidin-2-yl amino)-N,2-dimethylbenzamide

To a solution of intermediate D (200 mg, 0.41 mmol) in THF (15 mL) were added 3-fluoro-5-(trifluoromethyl)aniline (74 mg, 0.41 mmol) and DIEA (159 mg, 1.23 mmol). The reaction mixture was heated to 85° C. overnight. The solvent was removed and the crude was purified by column chromatography (DCM:methanol, 100-0 to100:2) to give a white solid which was further purified by preparative HPLC to give 5-(4-((4-(3-(3-fluoro-5-(trifluoromethyl)phenyl) ureido)phenyl)(methyl) amino)pyrimidin-2-yl amino)-N,2-dimethylbenzamide as a white solid (12 mg, 5%). ¹H NMR (400 MHz, DMSO-d₆): δ ppm 2.30 (s, 3H), 2.76 (d, J=4.4 Hz, 3H), 3.48 (s, 3H), 5.97 (br s, 1H), 7.26 (m, 2H), 7.35 (d, J=8.8 Hz, 2H), 7.60 (m, 5H), 7.76 (s, 1H), 7.85 (d, J=6.8 Hz, 1H), 9.47 (s, 1H), 9.70 (s, 1H), 10.33 (s, 1H). LCMS (method B): 2.62 min [MH]⁺=568.3.

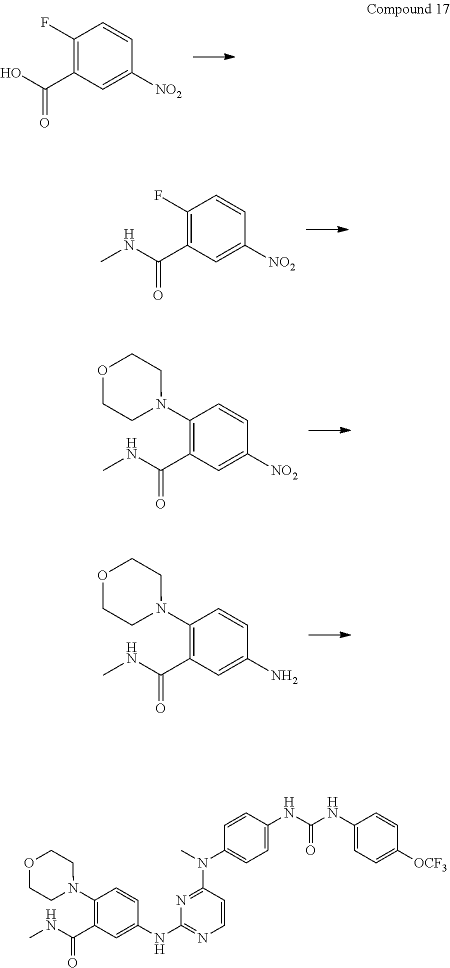 [SMILES: O=C(O)c1cc([N+](=O)[O-])ccc1F ; CNC(=O)c1cc([N+](=O)[O-])ccc1F ; CNC(=O)c1cc([N+](=O)[O-])ccc1N1CCOCC1 ; CNC(=O)c1cc(N)ccc1N1CCOCC1 ; CNC(=O)c1cc(Nc2nccc(N(C)c3ccc(NC(=O)Nc4ccc(OC(F)(F)F)cc4)cc3)n2)ccc1N1CCOCC1]

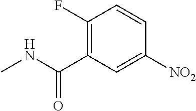

### Step 1: 2-fluoro-N-methyl-5-nitrobenzamide

To a solution of 2-fluoro-5-nitrobenzoic acid (1.0 g, 5.40 mmol) in dichloromethane (20 mL) were added HATU (2.05 g, 5.40 mmol), methylamine hydrochloride (400 mg, 5.94 mmol) and triethylamine (1.63 g, 16.2 mmol). The mixture was stirred at room temperature overnight. The mixture was diluted with dichloromethane (20 mL), washed with water and brine, dried over sodium sulfate and concentrated to dryness under reduced pressure to give 2-fluoro-N-methyl-5-nitrobenzamide (1.0 g, 93%) as a yellow solid. LCMS (method B): 2.07 min [MH]⁺=199.0.

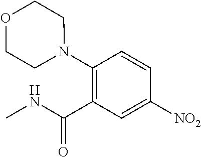 [SMILES: CNC(=O)c1cc([N+](=O)[O-])ccc1N1CCOCC1]

### Step 2: N-methyl-2-morpholino-5-nitrobenzamide

To a solution of 2-fluoro-N-methyl-5-nitrobenzamide (1.0 g, 5.05 mmol) in DMSO (10 mL) were added morpholine (530 mg, 6.06 mmol) and cesium carbonate (3.2 g, 10.1 mmol). The mixture was stirred at 80° C. overnight. The reaction mixture was poured into water (30 mL), and the resultant mixture was extracted with ethyl acetate (3×20 mL). The combined organic layers were washed with water and brine, dried over sodium sulfate and concentrated to dryness to give N-methyl-2-morpholino-5-nitrobenzamide (950 mg, 71%) as a yellow solid. LCMS (method B): 1.76 min [MH]⁺=266.1.

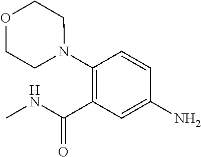 [SMILES: CNC(=O)c1cc(N)ccc1N1CCOCC1]

### Step 3: 5-amino-N-methyl-2-morpholinobenzamide

To a solution of N-methyl-2-morpholino-5-nitrobenzamide (950 mg, 3.58 mmol) in methanol (20 mL) was added palladium on carbon (10%, 60 mg), and the mixture was stirred at room temperature overnight under hydrogen atmosphere. The Pd/C was filtered off and the filtrate was concentrated to dryness under reduced pressure to give a residue which was purified by column chromatography (dichloromethane:methanol, 50:1) to give 5-amino-N-methyl-2-morpholinobenzamide (750 mg, 89% yield) as a light green solid. LCMS (method B): 1.23 min [MH]⁺=236.1.

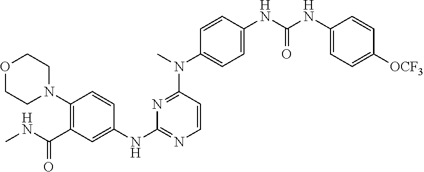 [SMILES: CNC(=O)c1cc(Nc2nccc(N(C)c3ccc(NC(=O)Nc4ccc(OC(F)(F)F)cc4)cc3)n2)ccc1N1CCOCC1]

### Step 4: N-methyl-5-((4-(methyl(4-(3-(4-(trifluoromethoxy) phenyl)ureido) phenyl)amino)pyrimidin-2-yl)amino)-2-morpholinobenzamide

To a solution of 5-amino-N-methyl-2-morpholinobenzamide (54 mg, 0.23 mmol) in isopropanol (8 mL) were added intermediate C (100 mg, 0.23 mmol) and HCl in dioxane (4 M, 2 drops). The resultant mixture was stirred at 85° C. overnight. The reaction mixture was concentrated to dryness to give a residue which was the purified by column chromatography (dichloromethane:methanol, 30:1) to give N-methyl-5-((4-(methyl(4-(3-(4-(trifluoromethoxy)phenyl)ureido) phenyl) amino)pyrimidin-2-yl)amino)-2-morpholinobenzamide (120 mg, 83%) as a white solid. ¹H NMR (400 MHz, DMSO-d₆): δ ppm 2.84 (br s, 4H), 2.85 (s, 3H), 3.42 (s, 3H), 3.75-3.69 (m, 4H), 5.79 (d, J=5.9 Hz, 1H), 7.11 (d, J=8.8 Hz, 1H), 7.25 (d, J=8.8 Hz, 2H), 7.30 (d, J=8.8 Hz, 2H), 7.58 (m, 4H), 7.78 (dd, J=8.8 and 2.7 Hz, 1H), 7.86 (d, J=6.0 Hz, 1H), 8.20 (s, 1H), 8.87 (s, 1H), 8.93 (s, 1H), 9.17 (s, 1H), 9.32 (d, J=4.6 Hz, 1H). LCMS (method B): 2.59 min [MH]⁺=637.3.

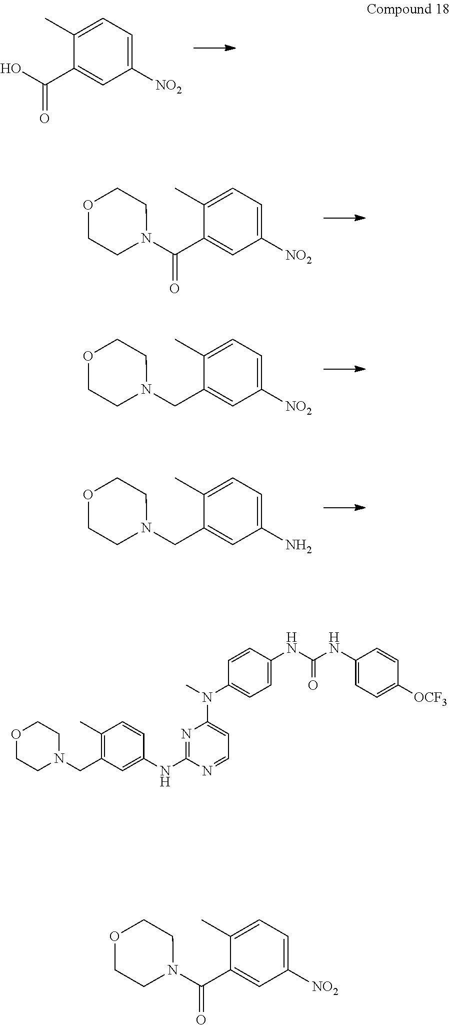 [SMILES: Cc1ccc([N+](=O)[O-])cc1C(=O)O ; Cc1ccc([N+](=O)[O-])cc1C(=O)N1CCOCC1 ; Cc1ccc([N+](=O)[O-])cc1CN1CCOCC1 ; Cc1ccc(N)cc1CN1CCOCC1 ; Cc1ccc(Nc2nccc(N(C)c3ccc(NC(=O)Nc4ccc(OC(F)(F)F)cc4)cc3)n2)cc1CN1CCOCC1 ; Cc1ccc([N+](=O)[O-])cc1C(=O)N1CCOCC1]

### Step 1: (2-methyl-5-nitrophenyl)(morpholino)methanone

2-methyl-5-nitrobenzoic acid (1 g, 5.5 mmol) was dissolved in SOCl₂ (15 mL), followed by addition of DMF (1 drop). The reaction mixture was refluxed for 4 h and then the solvent was removed under reduced pressure. DCM (10 mL) was added and concentrated to remove the excess of SOCl₂, then dissolved in DCM (5 mL) and cooled to 0° C. before the addition of a mixture of morpholine (529 mg, 6.08 mmol) and TEA (1.12 g, 11.16 mmol) in DCM (30 mL). The reaction mixture was stirred at room temperature overnight. LCMS analysis showed the reaction was complete. The mixture was diluted with DCM and washed with water (3×30 mL), dried, and concentrated to give (2-methyl-5-nitrophenyl)(morpholino)methanone as a yellow solid (1 g, 72%). LCMS (method B): 1.10 min [MH]⁺=251.1

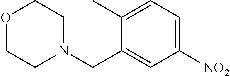 [SMILES: Cc1ccc([N+](=O)[O-])cc1CN1CCOCC1]

### Step 2: 4-(2-methyl-5-nitrobenzyl)morpholine

To a solution of (2-methyl-5-nitrophenyl)(morpholino)methanone (0.40 g, 1.63 mmol) in THF (20 mL) was added BH₃ in THF (1M, 8.2 mL, 8.15 mmol). The mixture was stirred at 60° C. under N₂ overnight. H₂O (50 mL) was added, followed by addition of ethyl acetate. The organic layer was separated, dried (Na₂SO₄). The organic layer was concentrated to give a residue which was purified by column chromatography on silica gel (DCM:MeOH, 250:1 to 200:1) to give 4-(2-methyl-5-nitrobenzyl)morpholine as a yellow solid (360 mg, 68%). ¹H NMR (400 MHz, DMSO-d₆): δ ppm 2.41 (m, 4H), 2.44 (s, 3H), 3.55 (s, 2H), 3.58 (m, 4H), 7.46 (d, J=8.4 Hz, 1H), 8.03 (dd, J=8.0 and 2.4 Hz, 1H), 8.13 (d, J=2.4 Hz, 1H). LCMS (method B): 2.43 min [MH]⁺=237.1.

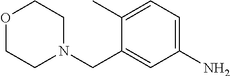

### Step 3: 4-methyl-3-(morpholinomethyl)aniline

To a solution of 4-(2-methyl-5-nitrobenzyl)morpholine (0.1 g, 0.42 mmol) in ethyl acetate (15 mL) was added Pd/C (10%, 20 mg) at room temperature. The reaction mixture was then stirred at room temperature overnight under a hydrogen atmosphere. Pd/C was filtered off and filtrate was concentrated to give 4-methyl-3-(morpholinomethyl) aniline as a yellow solid (70 mg, 79%). The crude product was used in the next step directly. LCMS (method B): 0.26 min [MH]⁺=207.1.

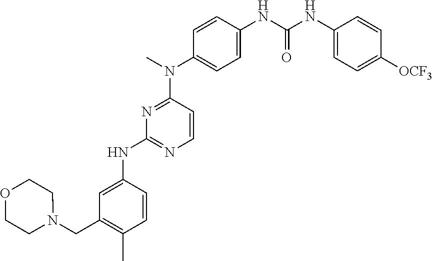 [SMILES: Cc1ccc(Nc2nccc(N(C)c3ccc(NC(=O)Nc4ccc(OC(F)(F)F)cc4)cc3)n2)cc1CN1CCOCC1]

### Step 4: 1-(4-(methyl(2-((4-methyl-3-(morpholinomethyl)phenyl)amino)pyrimidin-4-yl)amino)phenyl)-3-(4-(trifluoromethoxy)phenyl)urea

To a solution of intermediate C (149 mg, 0.34 mmol) in isopropyl alcohol (5 mL) were added 4-methyl-3-(morpholinomethyl)benzenamine (70 mg, 0.34 mmol) and concentrated HCl (0.5 mL). The mixture was stirred at 82° C. under N₂ overnight. The reaction mixture was diluted with water (10 mL), and extracted with ethyl acetate (3×10 mL). The organic layers were combined, dried (Na₂SO₄) and concentrated under reduced pressure. The crude oil was purified by column chromatography on silica gel (DCM:MeOH, 30:1) to give 1-(4-(methyl(2-((4-methyl-3-(morpholinomethyl)phenyl)amino)pyrimidin-4-yl)amino) phenyl)-3-(4-(trifluoromethoxy)phenyl)urea as a white solid (84 mg, 41%). ¹H NMR (400 MHz, DMSO-d₆): δ ppm 2.27 (s, 3H), 2.51 (m, 4H), 3.39 (s, 2H), 3.42 (s, 3H), 3.61 (br s, 4H), 5.77 (d, J=6 Hz, 1H), 7.04 (d, J=8.4 Hz, 1H), 7.28 (m, 4H), 7.50 (d, J=8.0 Hz, 1H), 7.58 (m, 4H), 7.72 (s, 1H), 7.83 (d, J=6.0 Hz, 1H), 9.10 (m, 3H). LCMS (method B): 2.36 min [MH]⁺=608.3.

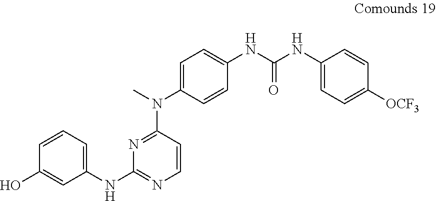 [SMILES: CN(c1ccc(NC(=O)Nc2ccc(OC(F)(F)F)cc2)cc1)c1ccnc(Nc2cccc(O)c2)n1]

### 1-(4-((2-(3-hydroxyphenylamino)pyrimidin-4-yl)(methyl)amino)phenyl)-3-(4-(trifluoromethoxy)phenyl)urea

Following general procedure C using intermediate C (120 mg, 0.274 mmol) and 3-aminophenol (30.5 mg, 0.28 mmol), 1-(4-((2-(3-hydroxyphenylamino) pyrimidin-4-yl)(methyl)amino)phenyl)-3-(4-(trifluoro-methoxy)phenyl)urea (8.6 mg, 6%) was obtained as a white solid. ¹H NMR (400 MHz, DMSO-d₆): δ ppm 3.43 (s, 3H), 5.79 (d, J=6.0 Hz, 1H), 6.38 (dd, J=8.0 Hz and 2.0 Hz, 1H), 7.02 (t, J=8.0 Hz, 1H), 7.14 (d, J=8.0 Hz, 1H), 7.28 (m, 5H), 7.59 (d, J=8.80 Hz, 4H), 7.84 (d, J=6.0 Hz, 1H), 9.16 (s, 1H), 9.20 (s, 1H), 9.28 (s, 2H). LCMS (method B): 2.54 min [M+H]⁺=511.2

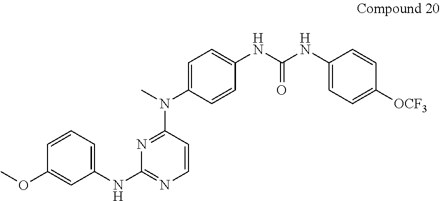 [SMILES: COc1cccc(Nc2nccc(N(C)c3ccc(NC(=O)Nc4ccc(OC(F)(F)F)cc4)cc3)n2)c1]

### 1-(4-((2-(3-methoxyphenylamino)pyrimidin-4-yl)(methyl)amino)phenyl)-3-(4-(trifluoromethoxy)phenyl)urea

Following general procedure C using intermediate C (120 mg, 0.274 mmol) and 3-methoxyaniline (38 mg, 0.28 mmol), 1-(4-((2-(3-methoxyphenylamino) pyrimidin-4-yl)(methyl)amino)phenyl)-3-(4-(trifluoromethoxy)phenyl)urea (66 mg, 46%) was obtained as a white solid. ¹H NMR (400 MHz, DMSO-d₆): δ ppm 3.43 (s, 3H), 3.72 (s, 3H), 5.78 (d, J=6.0 Hz, 1H), 6.48 (dd, J=8.0 Hz and 1.6 Hz, 1H), 7.12 (t, J=8.0 Hz, 1H), 7.32 (m, 5H), 7.59 (m, 5H), 7.87 (d, J=6.0 Hz, 1H), 8.83 (s, 1H), 8.93 (s, 1H), 9.13 (s, 1H). LCMS (method B): 2.72 min [M+H]⁺=525.2

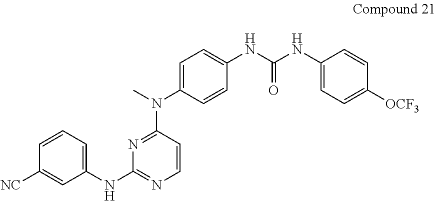 [SMILES: CN(c1ccc(NC(=O)Nc2ccc(OC(F)(F)F)cc2)cc1)c1ccnc(Nc2cccc(C#N)c2)n1]

Following general procedure C using intermediate C (120 mg, 0.274 mmol) and 3-aminobenzonitrile (33 mg, 0.28 mmol), 1-(4-((2-(3-cyanophenylamino) pyrimidin-4-yl)(methyl)amino)phenyl)-3-(4-(trifluoromethoxy)phenyl)urea (79 mg, 56%) was obtained as a white solid. ¹H NMR (400 MHz, DMSO-d₆): δ ppm 3.42 (s, 3H), 5.89 (d, J=6.0 Hz, 1H), 7.32 (m, 5H), 7.42 (t, J=8.0 Hz, 1H), 7.59 (m, 4H), 7.94 (d, J=6.0 Hz, 1H), 7.98 (d, J=8.40 Hz, 1H), 8.28 (s, 1H), 8.87 (s,1H), 8.94 (s, 1H), 9.56 (s, 1H). LCMS (method B): 2.68 min [M+H]⁺=520.2.

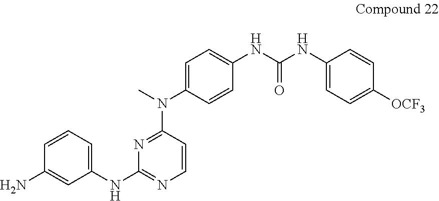 [SMILES: CN(c1ccc(NC(=O)Nc2ccc(OC(F)(F)F)cc2)cc1)c1ccnc(Nc2cccc(N)c2)n1]

Following general procedure C using intermediate C (120 mg, 0.274 mmol) and benzene-1,3-diamine (58 mg, 0.274 mmol), 1-(4-((2-(3-aminophenyl amino)pyrimidin-4-yl)(methyl)amino)phenyl)-3-(4-(trifluoromethoxy) phenyl)urea (33 mg, 21%) was obtained as a brown solid. ¹H NMR (400 MHz, DMSO-d₆): δ ppm 3.41 (s, 3H), 4.83 (s, 2H), 5.74 (d, J=6.0 Hz, 1H), 6.15 (d, J=7.6 Hz, 1H), 6.88 (m, 2H), 7.05 (s, 1H), 7.32 (m, 4H), 7.59 (m, 4H), 7.84 (d, J=6.0 Hz, 1H), 8.80 (s, 1H), 8.88 (s, 1H), 8.93 (s, 1H). LCMS (method B): 2.50 min [M+H]⁺=510.2.

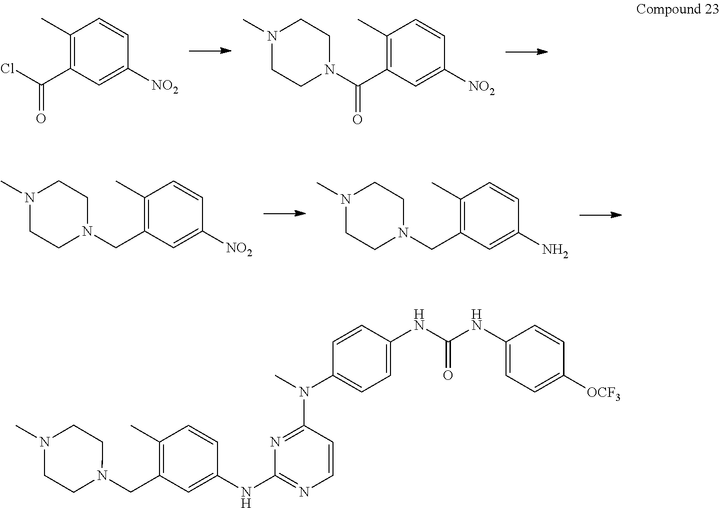 [SMILES: Cc1ccc([N+](=O)[O-])cc1C(=O)Cl ; Cc1ccc([N+](=O)[O-])cc1C(=O)N1CCN(C)CC1 ; Cc1ccc([N+](=O)[O-])cc1CN1CCN(C)CC1 ; Cc1ccc(N)cc1CN1CCN(C)CC1 ; Cc1ccc(Nc2nccc(N(C)c3ccc(NC(=O)Nc4ccc(OC(F)(F)F)cc4)cc3)n2)cc1CN1CCN(C)CC1]

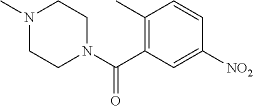 [SMILES: Cc1ccc([N+](=O)[O-])cc1C(=O)N1CCN(C)CC1]

### Step 1: (2-methyl-5-nitrophenyl)(4-methylpiperazin-1-yl)methanone

To a solution of 1-methylpiperazine (608 mg, 6.07 mmol) and TEA (1.12 g, 11 mmol) in DCM (15 mL) was added 2-methyl-5-nitrobenzoyl chloride (1.1 g, 5.52 mmol). The mixture was stirred for 3 hours at RT, and then diluted with water. The organic layer was washed with brine, dried over sodium sulfate and concentrated under reduced pressure to give (2-methyl-5-nitrophenyl)(4-methylpiperazin-1-yl)methanone (1 g, 69%) as a yellow solid. LCMS (method B): 0.30 min [MH]⁺=264.1.

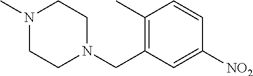 [SMILES: Cc1ccc([N+](=O)[O-])cc1CN1CCN(C)CC1]

### Step 2: 1-methyl-4-(2-methyl-5-nitrobenzyl) piperazine

To a solution of (2-methyl-5-nitrophenyl)(4-methylpiperazin-1-yl)methanone (544 mg, 2.07 mmol) in THF (10 mL) was added BH3.THF complex (1M, 10.33 mL, 10.33 mmol) at 0° C. The mixture was stirred under nitrogen atmosphere at 60° C. overnight cooled to RT and methanol (10 mL) was carefully added at 0° C. The reaction mixture was stirred for 4 h at RT, concentrated under reduced pressure and the residue was purified by column chromatography on silica gel (petroleum ether/ethyl acetate, 4:1) to give 1-methyl-4-(2-methyl-5-nitrobenzyl) piperazine (329 mg, 64%) as a yellow solid. LCMS (method B): 0.67 min [MH]⁺=250.1.

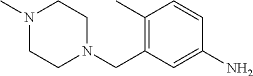 [SMILES: Cc1ccc(N)cc1CN1CCN(C)CC1]

### Step 3: 4-methyl-3-((4-methylpiperazin-1-yl)methyl) benzenamine

To a solution of 1-(2-methyl-5-nitrobenzyl)-4-methylpiperazine (329 mg, 1.32 mmol) in ethyl acetate (10 mL) was added Pd/C (40 mg). The mixture was stirred under an hydrogen atmosphere at RT for 16 hours. The Pd/C was removed via filtration, and the filtrate was concentrated under reduced pressure and purified by column chromatography on silica gel (eluent: DCM/MeOH=80:1 to 20:1) to give 4-methyl-3-((4-methylpiperazin-1-yl)methyl) benzenamine (143 mg, 49.4%) as a yellow solid. LCMS (method B): 0.26 min [MH]+=220.2.

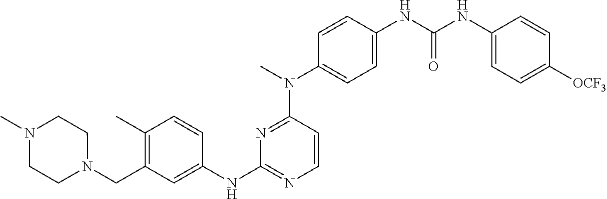 [SMILES: Cc1ccc(Nc2nccc(N(C)c3ccc(NC(=O)Nc4ccc(OC(F)(F)F)cc4)cc3)n2)cc1CN1CCN(C)CC1]

### Step 4: 1-(4-(methyl(2-((4-methyl-3-((4-methylpiperazin-1-yl)methyl)phenyl) amino)pyrimidin-4-yl) amino)phenyl)-3-(4-(trifluoromethoxy) phenyl)urea

To a solution of 4-methyl-3-((4-methylpiperazin-1-yl)methyl) benzenamine (133 mg, 0.61 mmol) in isopropanol (5 mL) was added intermediate C (265.5 mg, 0.61 mmol) and concentrated HCl (3 drops). The mixture was stirred at 85° C. overnight. The reaction mixture was poured into 1 M NaOH and extracted with ethyl acetate. The organic phase was washed with water, brine, dried and concentrated. The crude compound was purified by column chromatography on silica gel (DCM/MeOH, 40:1 to 10:1) to give 1-(4-(methyl(2-((4-methyl-3-((4-methylpiperazin-1-yl)methyl)phenyl) amino)pyrimidin-4-yl) amino)phenyl)-3-(4-(trifluoromethoxy) phenyl)urea (21.6 mg, 5.7%) as a white solid. ¹H NMR (400 MHz, DMSO-d₆): δ ppm 2.35 (m, 5H), 2.79 (s, 3H), 2.96 (m, 4H), 3.51 (m, 5H), 3.84 (m, 2H), 5.94 (m, 1H), 7.60 (m, 12H), 9.46 (s, 2H), 10.61 (m, 1H). LCMS (method B): 2.35 min [MH]⁺=621.3.

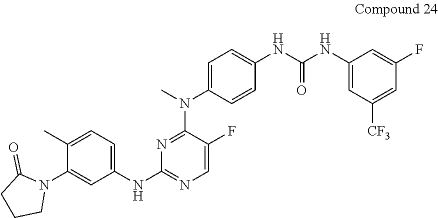 [SMILES: Cc1ccc(Nc2ncc(F)c(N(C)c3ccc(NC(=O)Nc4cc(F)cc(C(F)(F)F)c4)cc3)n2)cc1N1CCCC1=O]

Intermediate H (150 mg, 0.285 mmol), 3-fluoro-5 -(trifluoromethyl) benzenamine (51 mg, 0.285 mmol) and DMAP (21 mg, 0.171 mmol) were dissolved in THF (15 mL) and the reaction mixture was heated to 85° C. for 16 hours. The solvent was removed under reduced pressure and the crude was purified by column chromatography on silica gel (eluent DCM/Methanol 100:0 to 98:2) to give 1-(4-((5-fluoro-2-((4-methyl-3-(2-oxopyrrolidin-1-yl)phenyl) amino)pyrimidin-4-yl)(methyl)amino)phenyl)-3-(3-fluoro-5-(trifluoromethyl) phenyl)urea (29 mg, 17%) as a white solid. LCMS (method B): 3.11 min [MH]⁺=612.3. ¹H NMR (400 MHz, DMSO-d₆) δ ppm 2.06 (s, 3H) 2.11 (m, 2H) 2.42 (t, J=8.0 Hz 2H) 3.43 (s, 3H) 3.63 (t, J=7.2 Hz 2H) 7.13 (d, J=8.8 Hz 1H) 7.25 (m, 3H) 7.50 (m, 3H) 7.65 (m, 2H) 7.70 (s, 1H) 7.96 (d, J=5.6 Hz 1H) 9.16 (s, 1H) 9.29 (s, 1H) 9.50 (s, 1H).

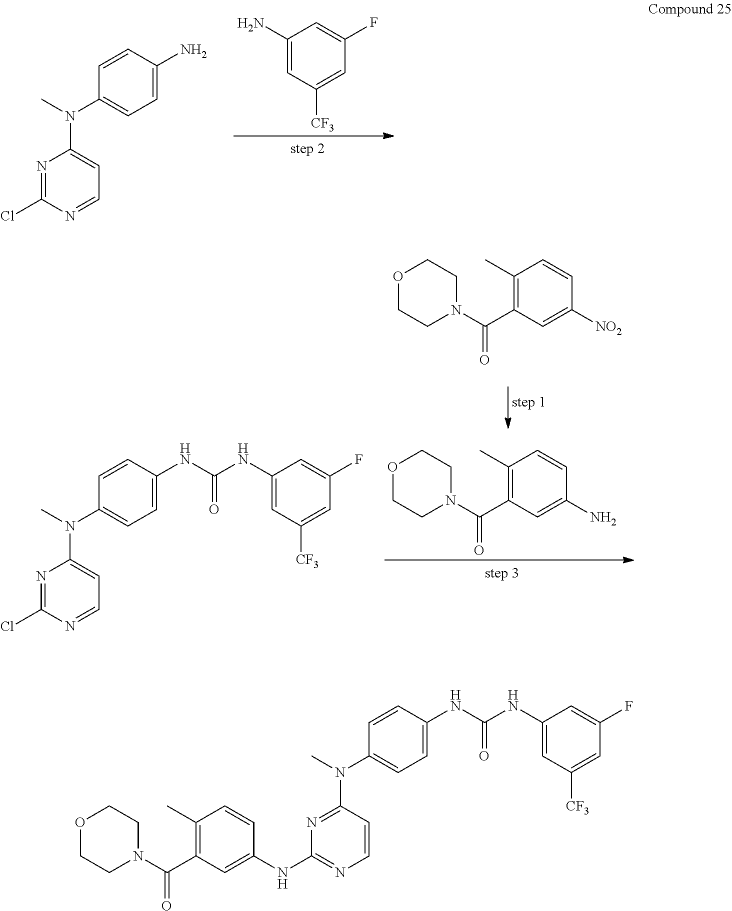

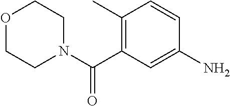 [SMILES: Cc1ccc(N)cc1C(=O)N1CCOCC1]

### Step 1: (5-amino-2-methylphenyl)(morpholino)methanone

To a solution of (2-methyl-5-nitrophenyl)(morpholino)methanone (step 1 of (18), (1.4 g 5.52 mmol) in methanol (40 mL) was added Pd/C (10%, 0.14 g) under N₂ atmosphere. The mixture was stirred at room temperature overnight under H₂ atmosphere. TLC and LCMS analysis showed the reaction was completed. The reaction mixture was filtered, concentrated under reduced pressure to afford (5-amino-2-methylphenyl)(morpholino)methanone (1.2 g, 100%) as a black solid. LCMS (method B): 0.31 min [MH]⁺=221.1

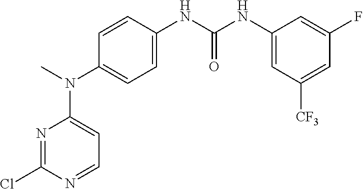 [SMILES: CN(c1ccc(NC(=O)Nc2cc(F)cc(C(F)(F)F)c2)cc1)c1ccnc(Cl)n1]

### Step 2: 1-(4-((2-chloropyrimidin-4-yl)(methyl)amino)phenyl)-3-(3-fluoro-5-(trifluoromethyl)phenyl)urea

To a solution of 3-fluoro-5-(trifluoromethyl)benzenamine (458 mg, 2.56 mmol) in DCM (20 mL) were added triethylamine (0.78 mL, 5.63 mmol) and a solution of triphosgene (249 mg, 0.84 mmol) in DCM (4 mL) dropwise. The resulting mixture was stirred at room temperature for 20 min, and then a solution of N1-(2-chloropyrimidin-4-yl)-N1-methylbenzene-1,4-diamine (from step 1 of the synthesis of intermediate C, 600 mg, 2.56 mmol) in DCM (30 mL) was added. The reaction mixture was stirred at room temperature for 16 hours and concentrated under reduced pressure. The residue was purified by column chromatography (eluent PE/EtOAc=80:20) to afford 1-(4-((2-chloropyrimidin-4-yl)(methyl)amino) phenyl)-3-(3-fluoro-5-(trifluoromethyl)phenyl)urea (305 mg, 27.2%) as a yellow solid. LCMS (method B): 3.18 min [MH]⁺=440.1.

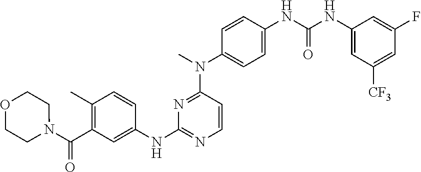 [SMILES: Cc1ccc(Nc2nccc(N(C)c3ccc(NC(=O)Nc4cc(F)cc(C(F)(F)F)c4)cc3)n2)cc1C(=O)N1CCOCC1]

### Step 3: 1-(3-fluoro-5-(trifluoromethyl)phenyl)-3-(4-(methyl(2-((4-methyl-3-(morpholine-4-carbonyl)phenyl)amino)pyrimidin-4-yl)amino)phenyl)urea

To a solution of (5-amino-2-methylphenyl)(morpholino)methanone (57 mg, 0.23 mmol) in isopropanol (15 mL) were added 1-(4-((2-chloropyrimidin-4-yl)(methyl)amino)phenyl)-3-(3-fluoro-5-(trifluoromethyl)phenyl)urea (100 mg, 0.23 mmol) and HCl (0.1 mL). The reaction mixture was stirred at room temperature for 16 hours and concentrated under reduced pressure. The resulting mixture was purified by column chromatography (eluent: DCM/MeOH: 99-1 to 96-4) to afford 1-(3-fluoro-5-(trifluoromethyl)phenyl)-3-(4-(methyl(2-((4-methyl-3-(morpholine-4-carbonyl)phenyl)amino)pyrimidin-4-yl)amino)phenyl)-urea (69 mg, 48.6%) as a yellow solid. LCMS (method B): 2.70 min [MH]⁺=624.3. ¹H NMR (400 MHz, DMSO-d₆) δ ppm 2.13 (s, 3H) 3.16 (brs, 2H), 3.39 (s, 3H) 3.50 (brs, 2H) 3.63 (brs, 4H) 5.77 (d, J=6.0 Hz, 1H) 7.11 (d, J=8.4 Hz, 1H) 7.20-7.32 (m, 3H) 7.57 (d, J=8.8 Hz, 2H) 7.63 (m, 3H) 7.73 (s, 1H) 7.87 (d, J=6.0 Hz, 1H) 9.07 (s, 1H) 9.18 (s, 1H) 9.32 (s, 1H).

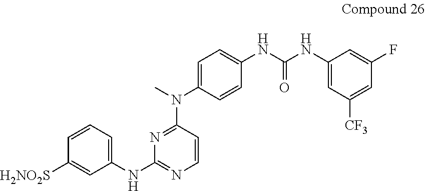 [SMILES: CN(c1ccc(NC(=O)Nc2cc(F)cc(C(F)(F)F)c2)cc1)c1ccnc(Nc2cccc(S(N)(=O)=O)c2)n1]

3-((4-((4-(3-(3-chloro-5-(trifluoromethyl)phenyl)ureido)phenyl)(methyl)amino) pyrimidin-2-yl)amino)benzenesulfonamide

Intermediate B (200 mg, 0.41 mmol), 3-chloro-5-(trifluoromethyl)benzenamine (80 mg, 0.41 mmol) and DIEA (106 mg, 2 mmol) were dissolved in THF (20 mL). The reaction mixture was heated to 85° C. for 18 hours. The solvent was removed under reduced pressure and the crude product was purified by column chromatography on silica gel (eluent DCM:methanol 100:0 to 98:2) to followed by pre-HPLC to give the titled compound (14 mg, 5.8%) as a white solid.

LCMS (method B): 2.66 min [MH]⁺=592.2. ¹H NMR (400 MHz, DMSO-d₆) δ ppm 3.45 (s, 3H) 5.81(d, J=6.4 Hz, 1H) 7.30 (m, 4H) 7.43 (m, 3H) 7.61 (d, J=8.8, 2H) 7.90 (m, 4H) 8.55 (s, 1H) 9.18 (s, 1H) 9.39 (s, 1H) 9.57 (s, 1H).

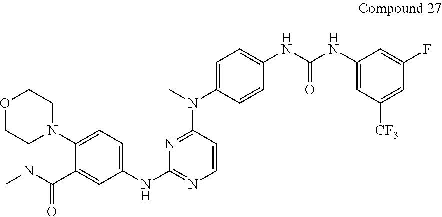 [SMILES: CNC(=O)c1cc(Nc2nccc(N(C)c3ccc(NC(=O)Nc4cc(F)cc(C(F)(F)F)c4)cc3)n2)ccc1N1CCOCC1]

### 5-((4-((4-(3-(3-fluoro-5-(trifluoromethyl)phenyl)ureido)phenyl)(methyl)amino) pyrimidin-2-yl)amino)-N-methyl-2-morpholinobenzamide

To a solution of 1-(4-((2-chloropyrimidin-4-yl)(methyl)amino)phenyl)-3-(3-fluoro-5-(trifluoromethyl) phenyl)urea (100 mg, 0.22 mmol) in i-PrOH (15 mL) were added 5-amino-N-methyl-2-morpholinobenzamide (from compound 17 step 3, 59 mg, 0.26 mmol) and concentrated HCl (2 drops). The reaction mixture was stirred at 85° C. overnight under nitrogen.The solvent was removed under reduced pressure and the crude product was purified by column chromatography on silica gel (DCM:MeOH, 100:1 to 40:1) to give a yellow solid which was further purified by pre-HPLC to afford the titled compound as a TFA salt (31 mg, 21%) as a white solid.

LCMS (method B): 2.69 min [MH]⁺=639.3. ¹H NMR (400 MHz, DMSO-d₆) δ ppm 2.85 (d J=4.8 Hz, 3H) 2.87 (br s 2H) 2.49 (br s 2H) 3.50 (s, 3H) 3.71 (br s 4H) 6.03 (br s, 1H) 7.27 (m, 2H) 7.38 (m 2H) 7.64 (m 7H) 9.13 (s, 1H) 9.60 (m 3H) 10.35 (s 1H).

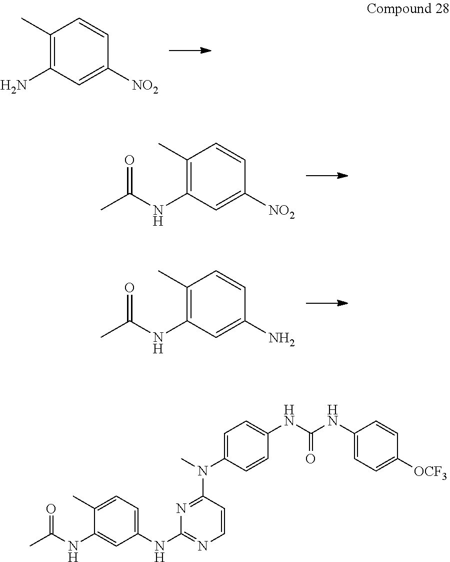 [SMILES: Cc1ccc([N+](=O)[O-])cc1N ; CC(=O)Nc1cc([N+](=O)[O-])ccc1C ; CC(=O)Nc1cc(N)ccc1C ; CC(=O)Nc1cc(Nc2nccc(N(C)c3ccc(NC(=O)Nc4ccc(OC(F)(F)F)cc4)cc3)n2)ccc1C]

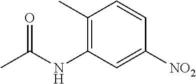

### Step 1: N-(2-methyl-5-nitrophenyl)acetamide

To a solution of 2-methyl-5-nitrobenzenamine (5.0 g, 0.033 mol) in DCM (80 mL) were added triethylamine (6.7 g, 0.066 mol) and acetyl chloride (2.9 g, 0.036 mol). The mixture was stirred at room temperature overnight. The reaction mixture was diluted with DCM (100 mL), and the organics were washed with water (200 mL), dried (Na₂SO₄), concentrated under reduced pressure and purified by column chromatography on silica gel (DCM:MeOH=160:1) to give N-(2-methyl-5-nitrophenyl)acetamide (2.2 g, 34%) as a yellow solid. LCMS (method B): 0.85 min [MH]⁺=195.1.

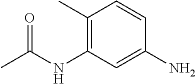

### Step 2: N-(5-amino-2-methylphenyl)acetamide

To a solution of N-(2-methyl-5-nitrophenyl)acetamide (1.0 g, 5.15 mmol) in MeOH (30 mL), were added Zinc powder (3.3 g, 51.5 mmol) and NH₄Cl (20 mL) aqueous saturated solution. The mixture was stirred at 60° C. for 4 hours. The reaction mixture was filtered to remove residual zinc powder and the filtrate was diluted with water (100 mL), extracted with ethyl acetate (200 mL), dried (Na₂SO₄), concentrated under reduced pressure and purified by column chromatography on silica gel (DCM:MeOH=80:1) to give N-(5-amino-2-methylphenyl)acetamide (390 mg, 46%) as a yellow solid. LCMS (method B): 0.21 min [MH]⁺=165.1.

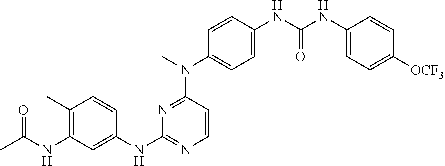 [SMILES: CC(=O)Nc1cc(Nc2nccc(N(C)c3ccc(NC(=O)Nc4ccc(OC(F)(F)F)cc4)cc3)n2)ccc1C]

### Step 3: N-(2-methyl-5-((4-(methyl(4-(3-(4-(trifluoromethoxy)phenyl)ureido) phenyl)amino)pyrimidin-2-yl)amino)phenyl)acetamide

To a solution of 1-(4-((2-chloropyrimidin-4-yl)(methyl)amino)phenyl)-3 -(4-(trifluoromethoxy) phenyl)urea (200 mg, 0.46 mmol) in isopropyl alcohol (40 mL) were added N-(5-amino-2-methylphenyl)acetamide (75 mg, 0.46 mmol) and conc HCl (0.1 mL). The mixture was stirred at 80° C. overnight. The reaction mixture was diluted with ethyl acetate (60 mL), washed with water (50 mL), dried (Na₂SO₄), concentrated under reduced pressure and purified by column chromatography on silica gel (DCM:MeOH=40:1) to give N-(2-methyl-5-((4-(methyl(4-(3-(4-(trifluoromethoxy)phenyl)ureido)phenyl)amino)pyrimidin-2-yl)amino)phenyl)acetamide (180 mg, 69%) as a white solid.

LCMS (method B): 2.69 min [MH]⁺=566.2.¹H NMR (400 MHz, DMSO-d₆): δ ppm 2.03 (s, 3H), 2.10 (s, 3H), 3.39 (s, 3H), 5.72 (d, J=6.0 Hz, 1H), 7.02 (d, J=8.4 Hz, 1H), 7.03-7.31 (m, 4H), 7.46-7.48 (m, 1H), 7.54-7.59 (m, 4H), 7.78 (s, 1H), 7.83 (d, J=5.6 Hz, 1H), 8.88 (s, 1H), 8.94 (s, 1H), 9.07 (s, 1H), 9.25 (s, 1H).

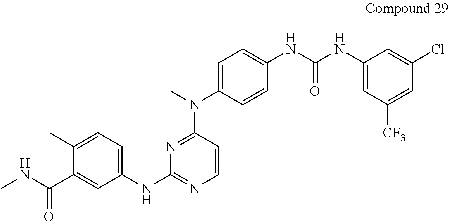 [SMILES: CNC(=O)c1cc(Nc2nccc(N(C)c3ccc(NC(=O)Nc4cc(Cl)cc(C(F)(F)F)c4)cc3)n2)ccc1C]

### 5-((4-((4-(3-(3-chloro-5-(trifluoromethyl)phenyl)ureido)phenyl)(methyl)amino) pyrimidin-2-yl) amino)-N,2-dimethylbenzamide

Intermediate E (120 mg, 0.25 mmol), 3-chloro-5-(trifluoromethyl)benzenamine (49 mg, 0.27 mmol) and DMAP (16 mg, 0.125 mmol) were dissolved in THF (15 mL). The reaction mixture was heated to 85° C. for 16 hours. The solvent was removed and the crude product was purified by column chromatography (DCM:methanol 100/0 to 98/2) to give 5-((4-((4-(3-(3-chloro-5-(trifluoromethyl)phenyl)ureido)phenyl)(methyl)amino)pyrimidin-2-yl) amino)-N,2-dimethylbenzamide (38 mg, 26%) as a white solid.

LCMS (method B): 2.74 min [MH]⁺=584.1. ¹H NMR (400 MHz, DMSO-d₆) δ ppm 2.23 (s, 3H) 2.74 (d, J=4.8 Hz, 3H) 3.41 (s, 3H) 5.77 (d, J=7.0 Hz, 1H) 7.07 (d, J=8.4 Hz, 1H) 7.28 (d, J=8.8 Hz, 2H) 7.43 (s, 1H) 7.59 (d, J=8.8 Hz, 2H) 7.67 (dd, J=8.4 2.0 Hz, 1H) 7.87 (m, 4H) 8.11 (m, 1H) 9.15 (s, 1H) 9.19 (s, 1H) 9.37(s, 1H).

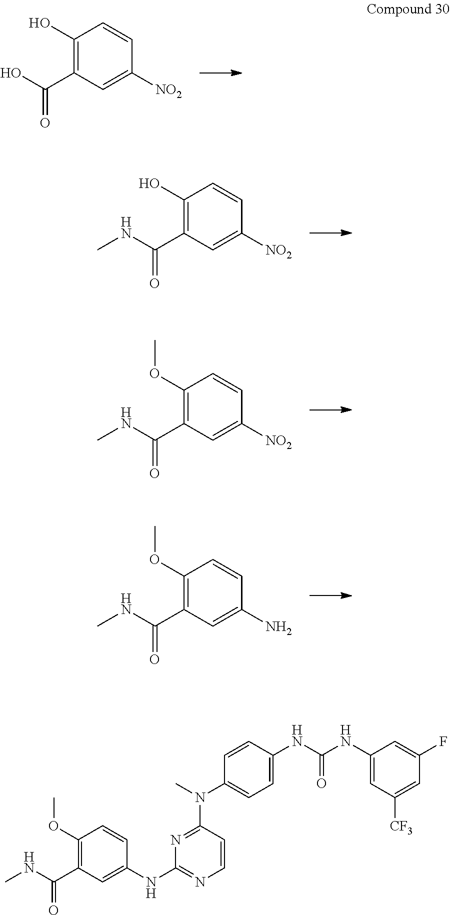 [SMILES: O=C(O)c1cc([N+](=O)[O-])ccc1O ; CNC(=O)c1cc([N+](=O)[O-])ccc1O ; CNC(=O)c1cc([N+](=O)[O-])ccc1OC ; CNC(=O)c1cc(N)ccc1OC ; CNC(=O)c1cc(Nc2nccc(N(C)c3ccc(NC(=O)Nc4cc(F)cc(C(F)(F)F)c4)cc3)n2)ccc1OC]

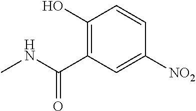

### Step 1: 2-hydroxy-N-methyl-5-nitrobenzamide

To a solution of 2-hydroxy-5-nitrobenzoic acid (700 mg,3.76 mmol) in DCM (40 mL) were added triethylamine (2.1 mL, 15.04 mmol) and HATU (1.56 g, 4.13 mmol). The resulting mixture was stirred at room temperature for 20 min, followed by addition of MeNH₂.HCl (304 mg, 4.52 mmol). The resulting mixture was stirred at room temperature for 16 hours. The reaction mixture was concentrated under reduced pressure and purified by column chromatography (PE/EtOAc=5/1 to 4/1) to afford the titled compound (130 mg, 17.3%) as a yellow solid. LCMS (method B): 1.59 min [MH]⁺=197.1

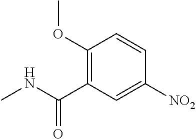

### Step 2: 2-methoxy-N-methyl-5-nitrobenzamide

To a solution of 2-hydroxy-N-methyl-5-nitrobenzamide (130 mg, 0.66 mmol) in DMF (8 mL) were added K₂CO₃ (230 mg, 1.66 mmol) foolowed by methyliodide (0.1 mL, 1.66 mmol). The resulting mixture was stirred at 40° C. for 16 hours. Water (10 mL) was added and the organic phase was extracted with ethyl acetate (3×10 mL). The combined organic layers were washed with brine (10 mL), dried over anhydrous sodium sulfate and concentrated under reduced pressure to afford 2-methoxy-N-methyl-5-nitrobenzamide (113 mg, 81%) as a yellow solid. LCMS (method B):0.73 min [MH]⁺=211.1

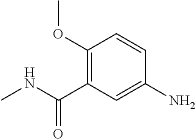

### Step 3: 5-amino-2-methoxy-N-methylbenzamide

To a solution of 2-methoxy-N-methyl-5-nitrobenzamide (113 mg, 0.53 mmol) in methanol (10 mL) were added zinc (344 mg, 52.90 mmol) and saturated ammonium chloride aqueous solution (5 mL). The reaction mixture was stirred at room temperature for 16 hours. The resulting mixture was filtered and the filtrate was concentrated under reduced pressure. The crude oil was partitioned between saturated sodium carbonate aqueous solution (5 mL) and ethyl acetate (5 mL). The organics were extracted with EtOAc (2×25 mL) and the combined organics were dried over anhydrous sodium sulfate, filtere and concentracted to afford 5-amino-2-methoxy-N-methylbenzamide (97 mg, 100%) as a yellow solid. LCMS (method B):0.72 min [MH]⁺=181.1.

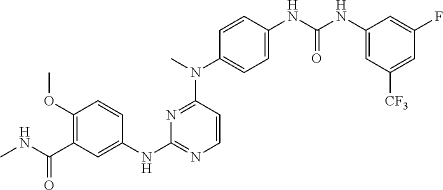 [SMILES: CNC(=O)c1cc(Nc2nccc(N(C)c3ccc(NC(=O)Nc4cc(F)cc(C(F)(F)F)c4)cc3)n2)ccc1OC]

### Step 4: 5-((4-((4-(3-(3-fluoro-5-(trifluoromethyl)phenyl)ureido)phenyl)(methyl) amino)pyrimidin-2-yl)amino)-2-methoxy-N-methylbenzamide

To a solution of 1-(4-((2-chloropyrimidin-4-yl)(methyl)amino)phenyl)-3-(3-fluoro-5-(trifluoromethyl) phenyl)urea (step 2 compound 25, 80 mg, 0.18 mmol) in isopropanol (15 mL) were added 5-amino-2-methoxy-N-methylbenzamide (32 mg, 0.18 mmol) and HCl (0.1 mL). The mixture was stirred at 85° C. for 16 hours. 5-amino-2-methoxy-N-methylbenzamide (16 mg, 0.09 mmol) was then added and the mixture was stirred at 85° C. for 24 hours. The mixture was concentrated under reduced pressure and the residue was purified by Preparative TLC (DCM:MeOH=10:1) to afford 5-((4-((4-(3-(3-fluoro-5-(trifluoromethyl)phenyl)ureido) phenyl) (methyl)amino)pyrimidin-2-yl)amino)-2-methoxy-N-methylbenzamide (41 mg, 40%) as a yellow solid.

LCMS (method B): 2.63 min [MH]⁺=584.3 1H NMR (400 MHz, DMSO-d6) δ ppm 2.78 (d, J=4.4 Hz, 3H), 3.41 (s, 3H), 3.82 (s, 3H), 5.75 (d, J=5.6 Hz, 1H), 6.99 (d, J=8.8 Hz, 1H), 7.28-7.20 (m, 3H), 7.57 (d, J=8.4 Hz, 2H), 7.70-7.62 (m, 2H), 7.85-7.77 (m, 2H), 8.19-8.09 (m, 2H), 9.10 (s, 1H), 9.52 (s, 1H), 9.94 (s, 1H).

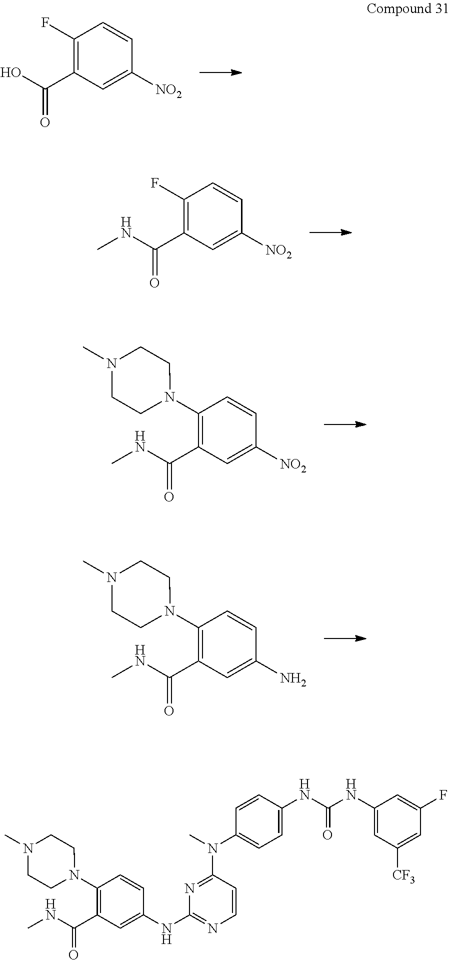 [SMILES: O=C(O)c1cc([N+](=O)[O-])ccc1F ; CNC(=O)c1cc([N+](=O)[O-])ccc1F ; CNC(=O)c1cc([N+](=O)[O-])ccc1N1CCN(C)CC1 ; CNC(=O)c1cc(N)ccc1N1CCN(C)CC1 ; CNC(=O)c1cc(Nc2nccc(N(C)c3ccc(NC(=O)Nc4cc(F)cc(C(F)(F)F)c4)cc3)n2)ccc1N1CCN(C)CC1]

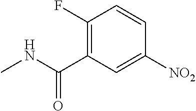

### Step1: 2-fluoro-N-methyl-5-nitrobenzamide

To a solution of 2-fluoro-5-nitrobenzoic acid (3 g, 16.2 mmol) in DCM (100 mL) were added TEA (8.9 mL, 64.8 mmol) and HATU (6.8 g, 17.8 mmol). The resulting mixture was stirred at room temperature for 20 min, followed by addition of MeNH₂.HCl (1.3 g, 19.4 mmol). The resulting mixture was stirred at room temperature overnight. The reaction mixture was washed with H₂O (3×30 mL), brine (30 mL), dried over anhydrous Na₂SO₄, filtered and concentrated under reduced pressure to afford the titled compound (3.2 g, contaminated with some solvent) as a yellow solid. LCMS (method B): 0.55 min [MH]⁺=199.0

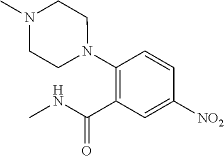 [SMILES: CNC(=O)c1cc([N+](=O)[O-])ccc1N1CCN(C)CC1]

### Step 2: N-methyl-2-(4-methylpiperazin-1-yl)-5-nitrobenzamide

To a solution of 2-fluoro-N-methyl-5-nitrobenzamide in DMSO (20 mL) were added Cs₂CO₃ (3.3 g, 10.10 mmol) and 1-methylpiperazine (0.73 mL, 6.57 mmol). The resulting mixture was stirred at 85° C. overnight. The reaction mixture was filtered and the filtrate was poured into water (30 mL). The resulting mixture was extracted with dichloromethane (3×20 ml). The organic layer was washed with brine (30 mL), dried over anhydrous sodium sulfate, concentrated under reduded pressure to afford crude N-methyl-2-(4-methylpiperazin-1-yl)-5-nitrobenzamide (1.4 g, 50%) as a yellow solid which was used in next step directly.

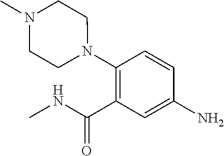 [SMILES: CNC(=O)c1cc(N)ccc1N1CCN(C)CC1]

### Step 3: 5-amino-N-methyl -2-(4-methylpiperazin-1-yl) benzamide

To a solution of the crude product from step 2 (1.4 g, 5.05 mmol) in methanol (100 mL) were added Zn (3.4 g, 52.90 mmol) and saturated NH₄Cl aqueous solution (25 mL). The reaction mixture was stirred at room temperature overnight. The TLC and LCMS analyses showed the reaction was completed. The resulting mixture was filtered, concentrated to give a residue. To the crude product was added saturated Na₂CO₃ aqueous solution (20 mL), and the resultant mixture was extracted with ethyl acetate (3*10 mL). The organic layer was washed with brine (10 mL), dried over anhydrous Na₂SO₄, filtered. The filtrate was concentrated and purified by column chromatography (DCM:MeOH=40:1˜20:1) to afford 5-amino-N-methyl-2-(4-methylpiperazin-1-yl) benzamide (260 mg, 20.8%) as a yellow solid. LCMS (method B): 0.28 min [MH]⁺=249.2.

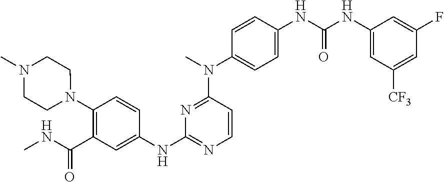 [SMILES: CNC(=O)c1cc(Nc2nccc(N(C)c3ccc(NC(=O)Nc4cc(F)cc(C(F)(F)F)c4)cc3)n2)ccc1N1CCN(C)CC1]

### Step 4: 5-((4-((4-(3-(3-fluoro-5-(trifluoromethyl)phenyl)ureido) phenyl)(methyl) amino)pyrimidin-2-Aamino)-N-methyl-2-(4-methylpiperazin-1-yl)benzamide

To a solution of (5-amino-2-methylphenyl)(morpholino)methanone (57 mg, 0.23 mmol) in isopropanol (15 mL) were added 5-amino-N-methyl-2-(4-methylpiperazin-1-yl)benzamide (100 mg, 0.23 mmol) and HCl (0.1 mL). The TLC and LCMS analyses showed the reaction was completed. The resulting mixture was purified by column chromatography (DCM:MeOH=60:1-40:1) to afford the titled compound (100 mg, 61.6%) as a yellow solid.

LCMS (method B): 2.34 min [MH]⁺=652.3 ¹H NMR (400 MHz, DMSO-d₆) δ ppm 2.19 (s, 3H) 2.43 (brs, 4H) 2.82 (brs, 4H) 2.83 (s, 3H) 3.41 (s, 3H) 5.80 (d, J=6.0 Hz, 1H) 7.09 (d, J=8.8 Hz, 1H) 7.27 (m, 3H) 7.58 (d, J=8.8 Hz, 2H) 7.66 (d, J=11.2 Hz, 1H) 7.75 (m, 2H) 7.86 (d, J=6.0 Hz, 1H) 8.20 (s, 1H) 9.07 (s, 1H) 9.17 (s, 1H) 9.32 (s, 1H) 9.52 (d, J=4.4 Hz, 1H).

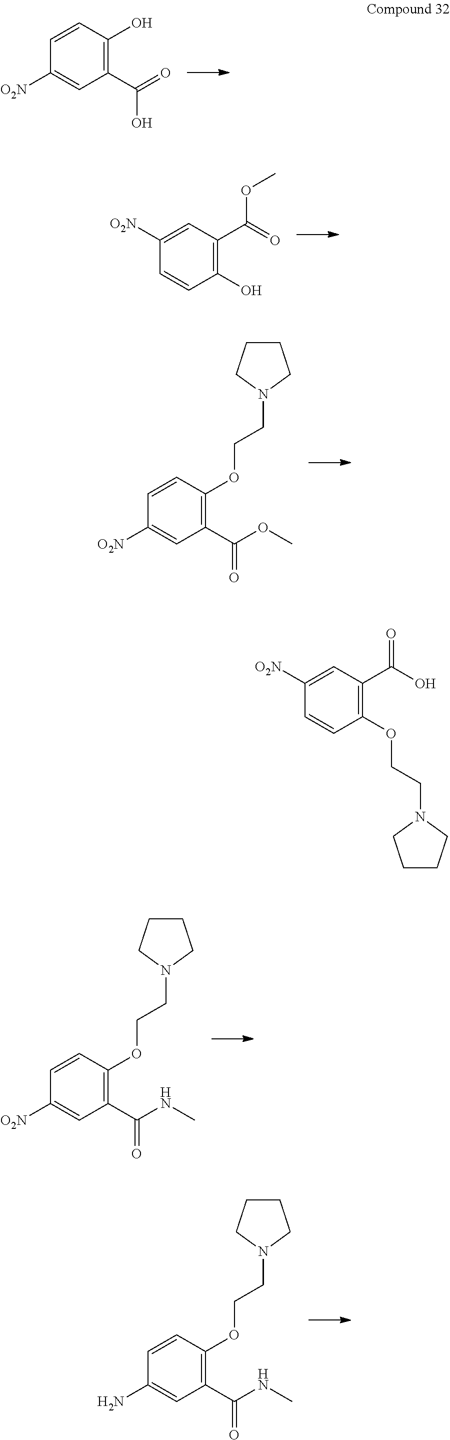 [SMILES: O=C(O)c1cc([N+](=O)[O-])ccc1O ; COC(=O)c1cc([N+](=O)[O-])ccc1O ; COC(=O)c1cc([N+](=O)[O-])ccc1OCCN1CCCC1 ; O=C(O)c1cc([N+](=O)[O-])ccc1OCCN1CCCC1 ; CNC(=O)c1cc([N+](=O)[O-])ccc1OCCN1CCCC1 ; CNC(=O)c1cc(N)ccc1OCCN1CCCC1]

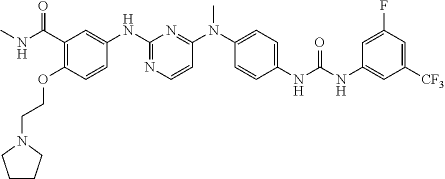 [SMILES: CNC(=O)c1cc(Nc2nccc(N(C)c3ccc(NC(=O)Nc4cc(F)cc(C(F)(F)F)c4)cc3)n2)ccc1OCCN1CCCC1]

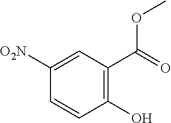

### Step 1: Methyl 2-hydroxy-5-nitrobenzoate

To a solution of 2-hydroxy-5-nitrobenzoic acid (2 g, 10.9 mmol) in methanol (40 mL) was added sulfuric acid (0.193 mL). The resulting mixture was stirred at 65° C. for 96 h. More sulfuric acid (0.193 mL) was added and the mixture was stirred at 80° C. for 40 h. Cooled to RT, the reaction mixture was concentrated under reduced pressure and dichloromethane (30 mL) was added. The organic layer was washed with saturated NaHCO₃ aqueous solution (3×10 mL), brine (10 mL), dried over anhydrous Na₂SO₄ and concentrated under reduced pressure to afford the titled compound (1.61 g, 75%) as a white solid. LCMS (method B): 2.33 min [MH]⁺=197.9

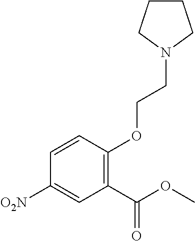 [SMILES: COC(=O)c1cc([N+](=O)[O-])ccc1OCCN1CCCC1]

### Step 2: Methyl 5-nitro-2 -(2-(pyrrolidin-1-yl)ethoxy)benzoate

To a solution of methyl 2-hydroxy-5-nitrobenzoate (700 mg, 3.55 mmol) in DMF (3 mL) were added 1-(2-chloroethyl)pyrrolidine (522 mg, 3.91 mmol), K₂CO₃ (981 mg, 7.10 mmol) and KI (589 mg, 3.55 mmol). The resulting mixture was stirred at 120° C. for 16 hours. The reaction mixture was partitioned between water and DCM (1/1, 20 mL) and the organics were extracted with dichloromethane (3*10 mL). The combined organic layers were washed with brine (10 mL), dried over anhydrous Na₂SO₄ and concentrated under reduced pressure. The residue was purified by column chromatography (DCM:MeOH=30:1 to 20:1) to afford methyl 5-nitro-2-(2-(pyrrolidin-1-yl)ethoxy)benzoate (180 mg, 17%) as a brown solid. LCMS (method B): 0.47 min [MH]⁺=295.1.

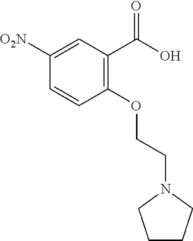 [SMILES: O=C(O)c1cc([N+](=O)[O-])ccc1OCCN1CCCC1]

### Step 3: 5-nitro-2-(2-(pyrrolidin-1-yl)ethoxy)benzoic acid

To a solution of methyl 5-nitro-2-(2-(pyrrolidin-1-yl)ethoxy)benzoate (180 mg, 0.61 mmol) in methanol (10 mL) were added sodium hydroxide (98 mg, 2.44 mmol) and water (10 mL). The mixture was stirred at RT for 1 h and then acidified to pH=3 with an aqueous solution of HCl 1M. The resulting mixture was concentrated under reduced pressure and filtered to afford 5-nitro-2-(2-(pyrrolidin-1-yl)ethoxy)benzoic acid (171 mg, 100%) as a brown solid. LCMS (method B): 0.34 min [MH]⁺=281.1

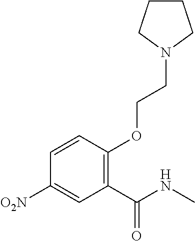 [SMILES: CNC(=O)c1cc([N+](=O)[O-])ccc1OCCN1CCCC1]

### Step 4: N-methyl-5-nitro-2-(2-(pyrrolidin-1-yl)ethoxy)benzamide

To a solution of 5-nitro-2-(2-(pyrrolidin-1-yl)ethoxy)benzoic acid (171 mg, 0.61 mmol) in dichloromethane (15 mL) were added triethylamine (0.34 mL, 2.44 mmol) and HATU (277 mg, 0.73 mmol). The resulting mixture was stirred at room temperature for 30 min, followed by addition of MeNH₂.HCl (49 mg, 0.73 mmol). The mixture was stirred at room temperature for 16 hours. DMF (6 mL) was added and the mixture was stirred at room temperature for 24 hours. The reaction mixture was washed with water (3×10 mL), HCl 1M (3 mL), brine (10 mL), dried over anhydrous Na₂SO₄, filtered, and concentrated to afford N-methyl-5-nitro-2-(2-(pyrrolidin-1-yl)ethoxy)benzamide (220 mg, contain some solvent) as a yellow solid. LCMS (method B): 0.30 min [MH]⁺=294.1

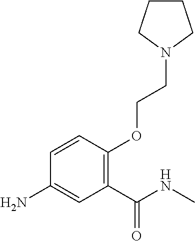 [SMILES: CNC(=O)c1cc(N)ccc1OCCN1CCCC1]

### Step 5: 5-amino-N-methyl-2-(2-(pyrrolidin-1-yl)ethoxy) benzamide

To a solution of N-methyl-5-nitro-2-(2-(pyrrolidin-1-yl)ethoxy)benzamide (220 mg, 0.75 mmol) in methanol/THF (20 mL/6 mL) was added Pd/C (22 mg). The resulting mixture was stirred at RT for 16 hours under H₂. The reaction mixture was filtered and concentrated under reduced pressure to afford 5-amino-N-methyl-2-(2-(pyrrolidin-1-yl)ethoxy) benzamide (200 mg, contain some solvent) as a yellow oil. LCMS (method B): 0.25 min [MH]⁺=264.1

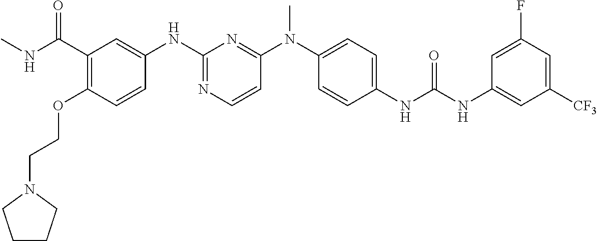 [SMILES: CNC(=O)c1cc(Nc2nccc(N(C)c3ccc(NC(=O)Nc4cc(F)cc(C(F)(F)F)c4)cc3)n2)ccc1OCCN1CCCC1]

### Step 6: 5-((4-((4-(3-(3-fluoro-5-(trifluoromethyl)phenyl)ureido)phenyl)(methyl) amino) pyrimidin-2-yl)amino)-N-methyl-2-(2-(pyrrolidin-1-yl)ethoxy)benzamide

To a solution of 5-amino-N-methyl-2-(2-(pyrrolidin-1-yl)ethoxy)benzamide (50 mg, 0.19 mmol) in isopropanol (6 mL) were added 1-(4-((2-chloropyrimidin-4-yl)(methyl)amino) phenyl)-3-(3-fluoro-5-(trifluoromethyl)phenyl)urea (84 mg, 0.19 mmol) and HCl (0.1 mL). The mixture was stirred at 85° C. for 16 hours. The reaction mixture was cooled to RT, filtered and purified by prep-TLC to afford 5-((4-((4-(3-(3-fluoro-5-(trifluoromethyl)phenyl)ureido)phenyl)(methyl) amino)pyrimidin-2-yl)amino)-N-methyl-2-(2-(pyrrolidin-1-yl)ethoxy)benzamide (20 mg, 16%) as a white solid.

LCMS (method B): 2.34 min [MH]⁺=667.3. ¹H NMR (400 MHz, DMSO-d₆) δ ppm 1.89 (br s, 2H), 2.04 (br s, 2H), 2.79 (d, J=4.4 Hz, 3H), 3.10 (br s, 2H), 3.47 (s, 3H), 3.62 (br s, 4H), 4.39 (br s, 2H), 5.92 (br s, 1H), 7.21 (m, 2H), 7.33 (d, J=8.4 Hz, 2H), 7.64 (m, 3H), 7.74 (s, 1H), 7.89 (m, 2H), 8.20 (m, 1H), 9.64 (s, 1H), 9.86 (s, 1H), 10.02 (br s, 1H), 10.40 (br s, 1H).

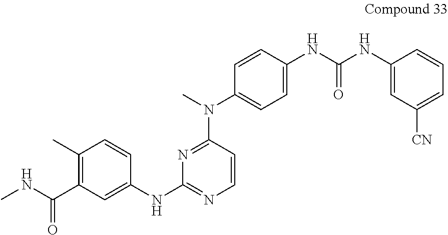 [SMILES: CNC(=O)c1cc(Nc2nccc(N(C)c3ccc(NC(=O)Nc4cccc(C#N)c4)cc3)n2)ccc1C]

### 5-((4-((4-(3-(3-cyanophenyl)ureido)phenyl)(methyl)amino)pyrimidin-2-yl)amino)-N,2-dimethylbenzamide

Intermediate E (120 mg, 0.25 mmol), 3-aminobenzonitrile (31 mg, 0.27 mmol) and DMAP (16 mg, 0.125 mmol) were dissolved in THF (15 mL). The reaction mixture was heated to 85° C. overnight. The solvent was removed and the crude product was purified by column chromatography (with eluent (DCM:methanol 100-100:2,v/v)) to afford the titled compound (20 mg, 16%) as a white solid. LCMS (method B): 2.29 min [MH]⁺=507.2. ¹H NMR (400 MHz, DMSO-d₆) δ ppm 2.22 (s, 3H) 2.73 (d, J=4.4 Hz, 3H) 3.40 (s, 3H) 5.76 (d, J=6.0 Hz, 1H) 7.07 (d, J=8.4 Hz, 1H) 7.28 (d, J=8.8 Hz, 2H) 7.44 (d, J=7.6 Hz, 1H) 7.53 (t, J=7.6 Hz, 1H) 7.58 (d, J=8.8 Hz, 2H) 7.70 (m, 2H) 7.87 (m, 2H) 8.00 (s, 1H) 8.08 (m, 1H) 9.03 (s, 1H) 9.13 (s, 1H) 9.17 (s, 1H).

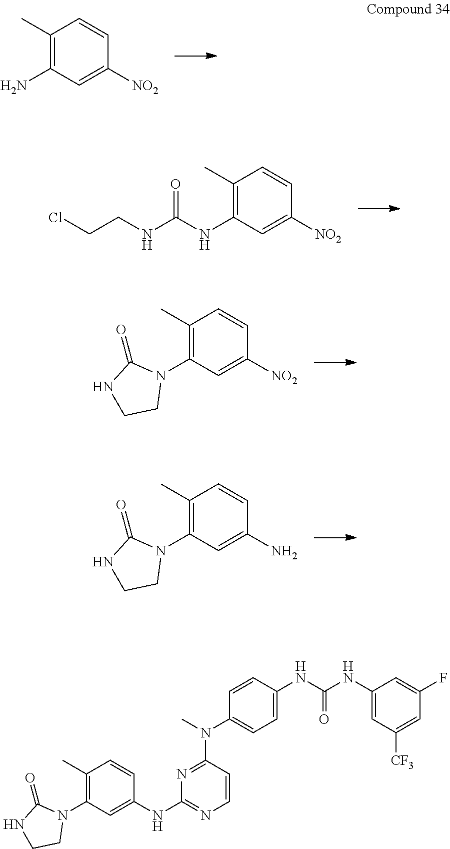 [SMILES: Cc1ccc([N+](=O)[O-])cc1N ; Cc1ccc([N+](=O)[O-])cc1NC(=O)NCCCl ; Cc1ccc([N+](=O)[O-])cc1N1CCNC1=O ; Cc1ccc(N)cc1N1CCNC1=O ; Cc1ccc(Nc2nccc(N(C)c3ccc(NC(=O)Nc4cc(F)cc(C(F)(F)F)c4)cc3)n2)cc1N1CCNC1=O]

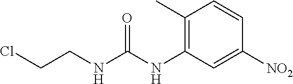 [SMILES: Cc1ccc([N+](=O)[O-])cc1NC(=O)NCCCl]

### Step 1: 1-(2-chloroethyl)-3-(2-methyl-5-nitrophenyl)urea

To a solution of 2-methyl-5-nitroaniline (2 g, 13.1 mmol) in THF (50 mL) was added 1-chloro-2-isocyanatoethane (2.08 g, 19.7 mmol) dropwise. The mixture was stirred at 70° C. for 16 hours. The reaction mixture was filtered and the solids were washed with water to give the desired product (1.24 g, 37%) as a yellow solid. LCMS (method B): 2.23 min [MH]⁺=258.1, min [MNa]⁺=280.1.

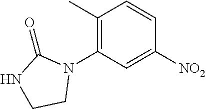 [SMILES: Cc1ccc([N+](=O)[O-])cc1N1CCNC1=O]

### Step 2: 1-(2-methyl-5-nitrophenyl)imidazolidin-2-one

To a solution of 1-(2-chloroethyl)-3-(2-methyl-5-nitrophenyl)urea (300 mg, 1.16 mmol) in THF (20 mL) was added sodium hydroxide (93 mg, 2.33 mmol) at 0° C. The mixture was stirred at room temperature for 1 hour. The reaction mixture was poured into water and was extracted with ethyl acetate. The organic phase was washed with brine, dried and concentrated under reduced pressure. The crude product was purified by column chromatography on silica gel (DCM:MeOH=80:1) to give the titled compound (183 mg, 53%) as a yellow solid. LCMS (method B): 0.27 min [MH]⁺=222.2, min [MNa]⁺=244.2.

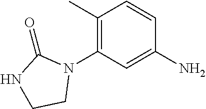

### Step 3: 1-(5-amino-2-methylphenyl)imidazolidin-2-one

To a solution of 1-(2-methyl-5-nitrophenyl)imidazolidin-2-one (120 mg, 0.54 mmol) in methanol (5 mL) and ammonium chloride solution (5 mL) was added zinc (350 mg, 5.4 mmol). The mixture was stirred at 60° C. for 2 hours and water was added. The organics were extracted with ethyl acetate (2×10 mL). The organic phase was washed with brine, dried (Na₂SO₄) and concentrated under reduced pressure to afford the titled compound (44 mg, 42%) as a yellow solid. LCMS (method B): 0.27 min [MH]⁺=192.2.

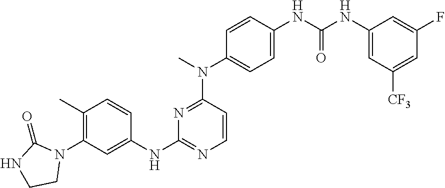 [SMILES: Cc1ccc(Nc2nccc(N(C)c3ccc(NC(=O)Nc4cc(F)cc(C(F)(F)F)c4)cc3)n2)cc1N1CCNC1=O]

### Step 4: 1-(3-fluoro-5-(trifluoromethyl)phenyl)-3-(4-(methyl(2-((4-methyl-3-(2-oxo imidazolidin-1-yl)phenyl)amino)pyrimidin-4-yl)amino)phenyl)urea

To a solution of 1-(5-amino-2-methylphenyl)imidazolidin-2-one (44 mg, 0.23 mmol) and 1-(4-((2-chloropyrimidin-4-yl)(methyl)amino)phenyl)-3-(3-fluoro-5-(trifluoromethyl) phenyl) urea (101.2 mg, 0.23 mmol) in isopropanol (10 mL) was added concentrated HCl (1 drop). The mixture was stirred at 85° C. for 16 hours. The reaction mixture was concentrated under redued pressure and the crude product was purified by column chromatography on silica gel (DCM:MeOH:ammonia=40:1:0.1) to give 40 mg of yellow solid. The solid was further washed with diethylether to give the titled compound (9 mg, 7%) as a yellow solid.

LCMS (method B): 1.99 min [MH]⁺=595.2. ¹HNMR (400 MHz, DMSO-d₆): δ ppm 2.13 (s, 3H), 3.40 (m, 5H), 3.67 (t, J=4.0 Hz, 2H), 5.76 (d, J=6.0 Hz, 1H), 6.56 (s, 1H), 7.08 (d, J=8.8 Hz, 1H), 7.26 (m, 4H), 7.5 (m, 1H), 7.57 (m, 2H), 7.65 (m, 1H), 7.69 (s, 1H), 7.86 (d, J=5.6 Hz, 1H), 9.07 (s, 1H), 9.09 (s, 1H), 9.30 (s, 1H).

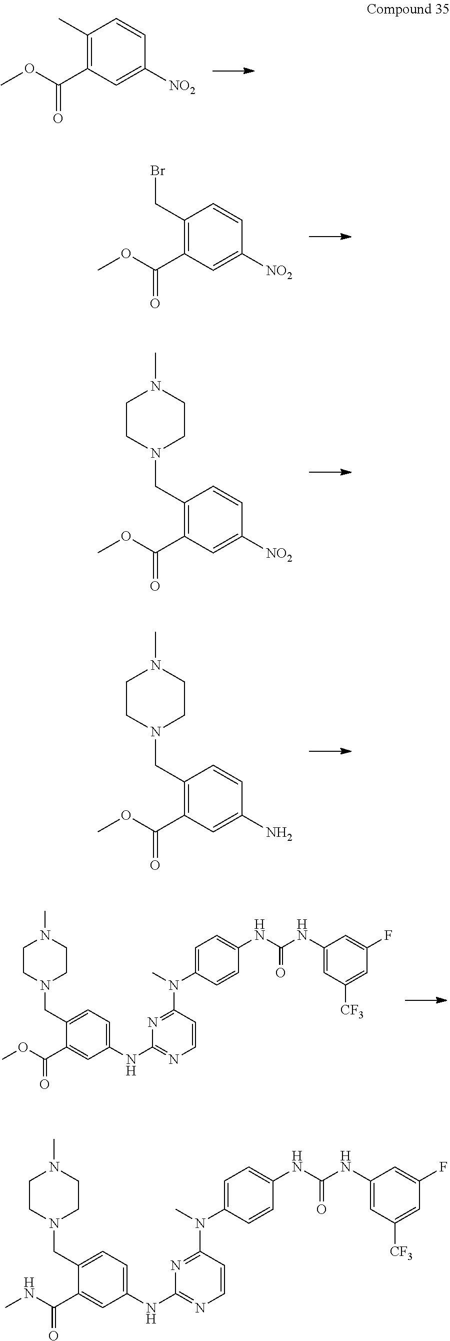 [SMILES: COC(=O)c1cc([N+](=O)[O-])ccc1C ; COC(=O)c1cc([N+](=O)[O-])ccc1CBr ; COC(=O)c1cc([N+](=O)[O-])ccc1CN1CCN(C)CC1 ; COC(=O)c1cc(N)ccc1CN1CCN(C)CC1 ; COC(=O)c1cc(Nc2nccc(N(C)c3ccc(NC(=O)Nc4cc(F)cc(C(F)(F)F)c4)cc3)n2)ccc1CN1CCN(C)CC1 ; CNC(=O)c1cc(Nc2nccc(N(C)c3ccc(NC(=O)Nc4cc(F)cc(C(F)(F)F)c4)cc3)n2)ccc1CN1CCN(C)CC1]

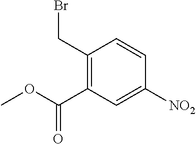

### Step 1: Methyl 2-(bromomethyl)-5-nitrobenzoate

To a solution of methyl 2-methyl-5-nitrobenzoate (6.4 g, 32.76 mmol) in CCl₄ (80 mL) were added NBS (6.4 g, 36.1 mmol) and BPO (794 mg, 3.28 mol). The resulting mixture was stirred at 83° C. for 5 h. The reaction mixture was concentrated under reduced pressure and the residue was diluted with EtOAc (150 mL), washed with H₂O (120 mL×3). The organic phase was dried and concentrated to give methyl 2-(bromomethyl)-5-nitrobenzoate (8.5 g, 95.5%) as a brown oil which was used directly for the next step without further purification. LCMS (method B): 2.59 min [M+Na]⁺=295.9,297.9

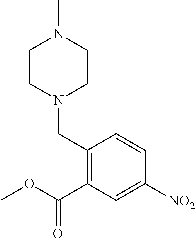 [SMILES: COC(=O)c1cc([N+](=O)[O-])ccc1CN1CCN(C)CC1]

### Step 2: methyl 2((4-methylpiperazin-1-yl)methyl)-5-nitrobenzoate

To a solution of methyl 2-(bromomethyl)-5-nitrobenzoate (1.39 g, 5.07 mmol) in CH₃CN (30 mL) were added 1-methyl piperazine (660 mg, 6.59 mmol) and K₂CO₃ (1.4 g, 10.14 mmol). The resulting mixture was stirred at 85° C. for 5 h under N₂. After cooling to RT, the mixture was diluted with H₂O and EtOAc and the aqueous layer was adjusted to pH 6 by addition of HCl 1M. The mixture was extracted with EtOAc (3×) and the combined organic layer were washed with brine, dried over Na₂SO₄, concentrated under reduced pressure. The residue was purified by column chromatography (CH₂Cl₂/MeOH=100/0 to 90/10) to give methyl 2-((4-methylpiperazin-1-yl)methyl)-5-nitrobenzoate (830 mg, 56%) as a brown oil. LCMS (method B): 0.52 min [MH]⁺=294.1

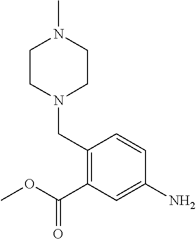 [SMILES: COC(=O)c1cc(N)ccc1CN1CCN(C)CC1]

### Step 3: methyl 5-amino-2-((4-methylpiperazin-1-yl)methyl)benzoate

To a solution of 2-((4-methylpiperazin-1-yl)methyl)-5-nitrobenzoic acid (417 mg, 1.42 mmol) in MeOH (9 mL) were added Zn (560 mg, 8.53 mmol) and NH₄Cl (saturated, aq, 3 mL). The resulting mixture was stirred at 60° C. for 3 h under N₂. After cooling to RT, the mixture was filtered and the filtrate was diluted with H₂O and CH₂Cl₂, and then was extracted with CH₂Cl₂. The combined organic layer was washed with brine, dried over Na₂SO₄, concentrated, to give methyl 5-amino-2-((4-methylpiperazin-1-yl)methyl)benzoate (260 mg, 69%) as a yellow oil which was used directly for the next step without further purification. LCMS (method B): 0.86 min [MH]⁺=264.2. MR-574-100.

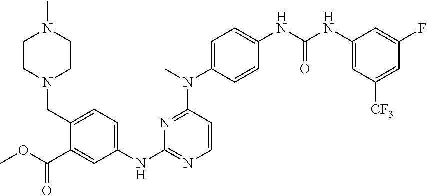 [SMILES: COC(=O)c1cc(Nc2nccc(N(C)c3ccc(NC(=O)Nc4cc(F)cc(C(F)(F)F)c4)cc3)n2)ccc1CN1CCN(C)CC1]

### Step 4: methyl 5-((4-((4-(3-(3-fluoro-5-(trifluoromethyl)phenyl)ureido)phenyl) (methyl)amino)pyrimidin-2-yl)amino)-2-((4-methylpiperazin-1-yl)methyl)-benzoate

5-amino-2-((4-methylpiperazin-1-yl)methyl)benzoic acid (30 mg, 0.11 mmol) and 1-(4-((2-chloropyrimidin-4-yl)(methyl)amino)phenyl)-3-(3-fluoro-5-(trifluoromethyl)phenyl)urea (50 mg, 0.11 mmol) were suspended in t-BuOH (5 mL) and conc.HCl (3 drops) was added. The resulting mixture was stirred at 55° C. under N₂ overnight. After cooling, the mixture was concentrated to give a residue which was purified by Pre.TLC (CH₂Cl₂/MeOH=15/1, v/v) to give the titled compound (35 mg, 47%) as an off-white solid. LCMS (method B): 2.46 min [MH]⁺=667.3

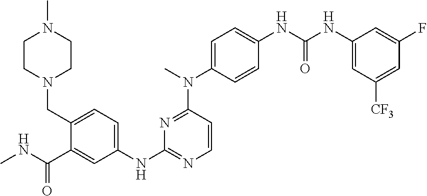 [SMILES: CNC(=O)c1cc(Nc2nccc(N(C)c3ccc(NC(=O)Nc4cc(F)cc(C(F)(F)F)c4)cc3)n2)ccc1CN1CCN(C)CC1]

### Step 5: 5-((4-((4-(3-(3-fluoro-5-(trifluoromethyl)phenyl)ureido)phenyl)(methyl) amino)pyrimidin-2-yl)amino)-N-methyl-2-((4-methylpiperazin-1-yl)methyl)benzamide

5-((4-((4-(3-(3-fluoro-5-(trifluoromethyl)phenyl)ureido)phenyl)(methyl)amino)pyrimidin-2-yl)am ino)-2-((4-methylpiperazin-1-yl)methyl)benzoic acid (35 mg, 0.05 mmol) was dissolved in CH₃NH₂ in EtOH (3 mL) in a sealed tube. The resulting mixture was stirred at 35° C. for 28 h. The reaction mixture was concentrated under reduced pressure and the residue was purified by PrepTLC (CH₂Cl₂/MeOH=13/1, v/v) to give the titled compound (12 mg, 34%) as a white solid.

LCMS (method B): 2.32 min [MH]⁺=666.3. ¹H NMR (400 MHz, DMSO) δ 11.47 (s, 1H), 11.02 (s, 1H), 9.50 (d, J=4.6 Hz, 1H), 9.26 (s, 1H), 8.04 (s, 1H), 7.85 (d, J=6.0 Hz, 1H), 7.82-7.68 (m, 3H), 7.64 (d, J=8.7 Hz, 2H), 7.22 (d, J=8.8 Hz, 2H), 7.13 (t, J=9.2 Hz, 2H), 5.78 (d, J=5.9 Hz, 1H), 3.41 (m, 5H), 2.78 (d, J=4.5 Hz, 3H), 2.35 (m, 8H), 2.13 (s, 3H).

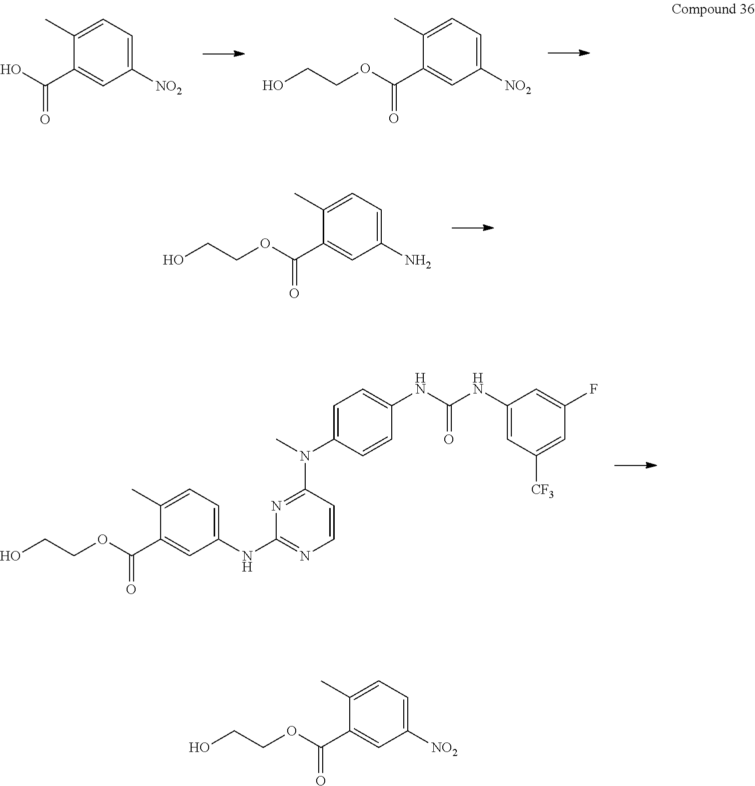 [SMILES: Cc1ccc([N+](=O)[O-])cc1C(=O)O ; Cc1ccc([N+](=O)[O-])cc1C(=O)OCCO ; Cc1ccc(N)cc1C(=O)OCCO ; Cc1ccc(Nc2nccc(N(C)c3ccc(NC(=O)Nc4cc(F)cc(C(F)(F)F)c4)cc3)n2)cc1C(=O)OCCO ; Cc1ccc([N+](=O)[O-])cc1C(=O)OCCO]

### Step 1: 2-hydroxyethyl 2-methyl-5-nitrobenzoate

To a solution of 2-methyl-5-nitrobenzoic acid (1 g, 5.52 mmol) in DCM (20 mL) were added triethylamine (1.69 mL, 12.14 mmol) and HATU (2.31 g, 6.07 mmol). The resulting mixture was stirred at room temperature for 30 min, followed by addition of ethylene glycol (343 mg, 5.52 mmol). The resulting mixture was stirred at RT for 16 hours. The reaction mixture was washed with water (3×10 mL), and the aqueous phases were back extracted with DCM (3×10 mL). The combined organic layers were washed with brine (10 mL), dried over anhydrous Na₂SO₄, filtered, and concentrated under reduce pressure. The residue was purified by column chromatography (PE:EtOAc=7:1 to 6:1) to afford the crude product (500 mg, 40%) as yellow oil. LCMS (method B): 2.05 min [MNa]⁺=248.0.

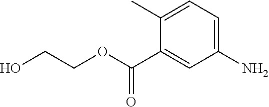

### Step 2: 2-hydroxyethyl 5-amino-2-methylbenzoate

To a solution of 2-hydroxyethyl 2-methyl-5-nitrobenzoate (500 mg, 2.22 mmol) in methanol (15 mL) were added zinc (1.43 g, 22.2 mmol) and saturated ammonium chloride aqueous solution (12 mL). The reaction mixture was stirred at room temperature for 2 h. The resulting mixture was filtered and the filtrate was concentrated under reduced pressure. The reaction mixture was partitioned between saturated Na₂CO₃ aqueous solution (12 mL) and dichloromethane and the aqueous layer was extracted with DCM (3×10 mL). The combined organic layers were washed with brine (10 mL), dried over anhydrous Na₂SO₄, filtered and concentrated under reduced pressure to afford crude titled product (438 mg, 100%) as yellow oil. LCMS (method B): 0.37 min [MH]⁺=196.1.

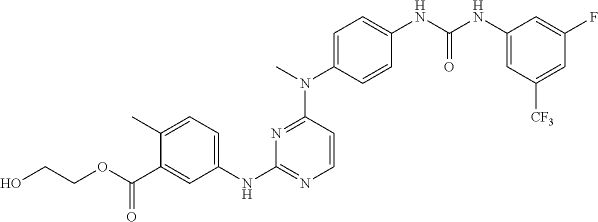 [SMILES: Cc1ccc(Nc2nccc(N(C)c3ccc(NC(=O)Nc4cc(F)cc(C(F)(F)F)c4)cc3)n2)cc1C(=O)OCCO]

### Step 3: 2-hydroxyethyl 5-((4-((4-(3-(3-fluoro-5-(trifluoromethyl)phenyl)ureido) phenyl)(methyl) amino)pyrimidin-2-yl)amino)-2-methylbenzoate

To a solution of 2-hydroxyethyl 5-amino-2-methylbenzoate (45 mg, 0.23 mmol) in isopropanol (10 mL) were added 1-(4-((2-chloropyrimidin-4-yl)(methyl)amino)phenyl)-3-(3-fluoro-5-(trifluoromethyl)phenyl)urea (100 mg, 0.23 mmol) and concentrated HCl (0.1 mL). The resulting mixture was stirred at 85° C. for 16 hours. The mixture was concentrated under reduced pressure and purified by column chromatography (DCM:MeOH:NH₃.H₂O=20:1:0.2) to afford 2-hydroxyethyl 5-((4-((4-(3-(3-fluoro-5-(trifluoromethyl)phenyl)ureido) phenyl)(methyl) amino)pyrimidin-2-yl)amino)-2-methylbenzoate (80 mg, 58%) as a yellow solid.

LCMS (method B): 3.37 min [MH]⁺=599.3. ¹H NMR (400 MHz, DMSO-d6) δ ppm 2.45 (s, 3H) 3.45 (s, 3H) 3.69 (br s, 2H) 4.26 (t, J=5.2 Hz 2H) 4.86 (br s, 1H) 5.79 (d, J=6 Hz, 1H) 7.18 (d, J=8.4 Hz 1H) 7.24 (d, J=8.4 Hz 1H) 7.29 (d, J=8.4 Hz, 2H) 7.61 (m, 3H) 7.71 (m, 2H), 7.86 (d, J=6.0 Hz, 1H) 8.44 (s, 1H) 9.16 (s, 1H) 9.38 (s, 1H) 9.42 (s, 1H).

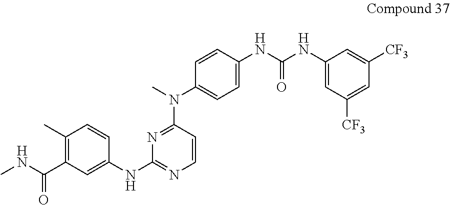 [SMILES: CNC(=O)c1cc(Nc2nccc(N(C)c3ccc(NC(=O)Nc4cc(C(F)(F)F)cc(C(F)(F)F)c4)cc3)n2)ccc1C]

### 5-((4-((4-(3-(3,5-bis(trifluoromethyl)phenyl)ureido)phenyl)(methyl)amino) pyrimidin-2-yl)amino)-N,2-dimethylbenzamide

To a solution of bis(trichloromethyl)carbonate (27 mg, 0.09 mmol) in anhydrous dichloromethane (3 mL) was added 2-chloro-5-(trifluoromethyl)aniline (64 mg, 0.28 mmol) and TEA (0.087 mL, 0.62 mmol) in anhydrous DCM (2 mL) dropwise at 0° C. The resulting mixture was stirred at 0° C. for 1 h. Then a solution of intermediate D (100 mg, 0.28 mmol) in DMF (4 mL) was added dropwise at 0° C. The resulting mixture was stirred at 0° C. for 16 hours. The reaction mixture was diluted with H₂O. The aqueous phase was extracted with DCM (3×2 mL). The combined organic layers were washed with brine (2 mL), dried over anhydrous Na₂SO₄, filtered, and concentrated under vaccum. The residue was purified by Prep-TLC (DCM:MeOH=10:1) to afford crude product (30 mg) which was purified by Prep-HPLC to afford the desired product as a TFA salt (17 mg, 10%) as a white solid.

LCMS (method B): 2.83 min [MH]⁺=618.2. ¹H NMR (400 MHz, DMSO-d₆) δ ppm 2.22 (s, 3H) 2.72 (d, J=4.4 Hz 3H) 3.40 (s, 3H) 5.76 (d, J=5.6 Hz 1H) 7.05 (d, J=8.4 Hz 1H) 7.26 (d, J=8.8 Hz 2H) 7.59 (d, J=8.8 Hz 2H) 7.65 (m, 2H) 7.81 (br s, 1H) 7.85 (d, J=6.0 Hz 1H) 8.06 (m,1H) 8.16 (br s, 2H) 9.15 (br s, 1H) 9.57 (s, 1H) 10.00 (br s, 1H).

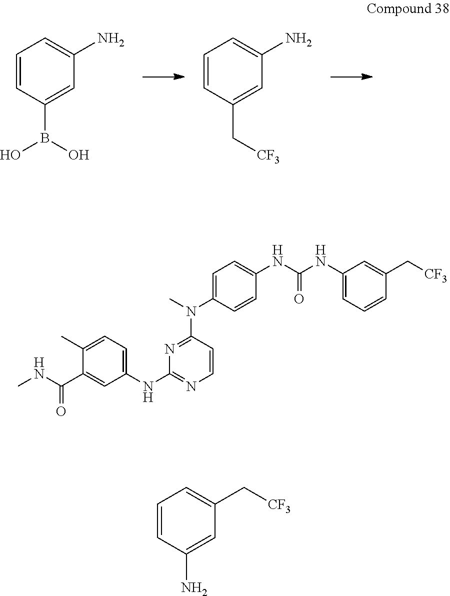 [SMILES: Nc1cccc(B(O)O)c1 ; Nc1cccc(CC(F)(F)F)c1 ; CNC(=O)c1cc(Nc2nccc(N(C)c3ccc(NC(=O)Nc4cccc(CC(F)(F)F)c4)cc3)n2)ccc1C ; Nc1cccc(CC(F)(F)F)c1]

### Step 1: 3-(2,2,2-trifluoroethyl)aniline

A mixture of (3-aminophenyl)boronic acid (200.0 mg, 1.5 mmol), Cs₂CO₃ (1.9 g, 6.9 mmol), Xantphos (143.0 mg, 0.24 mmol), Pd₂(dba)₃ (66.0 mg, 0.073 mmol) in dioxane (10 mL) were stirred for 10 min under N₂. Then a solution of 1,1,1-trifluoro-2-iodoethane (6.1 g, 4.9 mmol) and water (2 mL) were added. The mixture was stirred for 10 h at 80° C. Cooled to RT, the reaction mixture was filtered and the filtrate was concentrated under reduced pressure. The residue was partitioned between water (5 mL) and ethyl acetate (10 mL) and the organic layer was separated, dried over Na₂SO₄ and concentrated under reduced pressure. The residue was purified by column chromatography on silica gel (eluent: petroleum ether:ethyl acetate=10:1) to give the desired product (160.0 mg, 63%) as a yellow solid LCMS (method B): 3.26 min [MH]⁺=176.2.

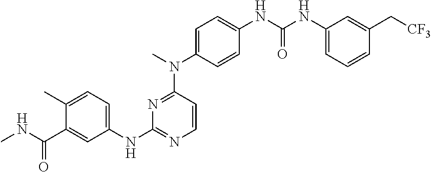 [SMILES: CNC(=O)c1cc(Nc2nccc(N(C)c3ccc(NC(=O)Nc4cccc(CC(F)(F)F)c4)cc3)n2)ccc1C]

### Step 2: N,2-dimethyl-5-((4-(methyl(4-(3-(3-(2,2,2-trifluoroethyl)phenyl)ureido) phenyl)amino)pyrimidin-2-yl)amino)benzamide

A mixture of 3-(2,2,2-trifluoroethyl) aniline (80.0 mg, 0.45 mmol), intermediate E (180.0 mg, 0.38 mmol), DMAP (3.0 mg, 0.02 mmol) and DIEA (116.0 mg, 0.9 mmol) in DMF (5 mL) was heated at 85° C. for 10 h under N₂. Water (10 mL) was added and the resulting mixture was extracted with ethyl acetate (2×10 mL). The combined organic layers were dried over Na₂SO₄ , filtered and concentrated under reduced pressure. The residue was purified by silca gel column chromatography (eluent: petroleum ether:ethyl acetate=2:1) to give the titled compound (80.0 mg, 32%) as a yellow solid.

LCMS (method B): 2.45 min [MH]⁺=564.3. ¹H NMR (400 MHz, DMSO-d6) δ 10.35-10.22 (br s, 1H), 9.04 (s, 1H), 8.96 (s, 1H), 8.15 (d, J=4.6 Hz, 1H), 7.83 (d, J=6.4 Hz, 1H), 7.63-7.61 (m, 3H), 7.52 (br s, 2H), 7.44 (d, J=8.1 Hz, 1H), 7.33-7.31 (m, 3H), 7.26-7.19 (m, 1H), 6.98 (d, J=7.6 Hz, 1H), 6.05-5.85 (m, 1H), 3.47 (s, 3H, covered), 2.75 (d, J=4.6 Hz, 3H), 2.54 (s, 3H), 2.29 (s, 2H).

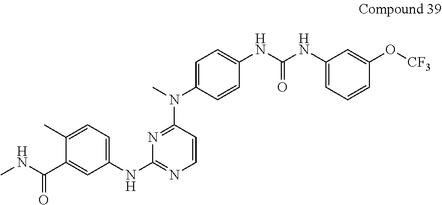 [SMILES: CNC(=O)c1cc(Nc2nccc(N(C)c3ccc(NC(=O)Nc4cccc(OC(F)(F)F)c4)cc3)n2)ccc1C]

### N,2-dimethyl-5-((4-(methyl(4-(3-(3-(trifluoromethoxy)phenyl)ureido)phenyl) amino)pyrimidin-2-yl)amino)benzamide

3-(trifluoromethoxy)aniline (50.0 mg, 0.28 mmol), intermediate E (80.0 mg, 0.24 mmol), DMAP (1.5 mg, 0.01 mmol) and DIEA (62.0 mg, 0.48 mmol) in DMF (5 mL) was heated at 85° C. for 10 h under N₂. Water (10 mL) was added and the mixture was extracted with ethyl acetate (2×10 mL). The combined organics were dried over Na₂SO₄ , filtered and concentrated under reduced pressure. The residue was purified by silica gel column chromatography (eluent: DCM:MeOH=15:1) to afford the titled compound (40.0 mg, 30%) as a yellow solid.

LCMS (method B): 2.66 min [MH]⁺=566.0. ¹H NMR (400 MHz, DMSO-d6) δ 9.53 (s, 2H), 9.44-9.41 (m, 2H), 8.10-8.09 (m, 1H), 7.80-7.78 (m, 2H), 7.64 (dd, J=8.4, 2.0 Hz, 1H), 7.32 (br s, 1H), 7.20 (br s, 1H), 7.12-7.07 (m, 2H), 6.96 (d, J=8.4 Hz, 2H), 6.65 (d, J=8.4 Hz, 2H), 5.70 (d, J=6.0 Hz, 1H), 3.43 (s, 3H, covered), 2.74 (d, J=3.9 Hz, 3H), 2.25 (s, 3H).

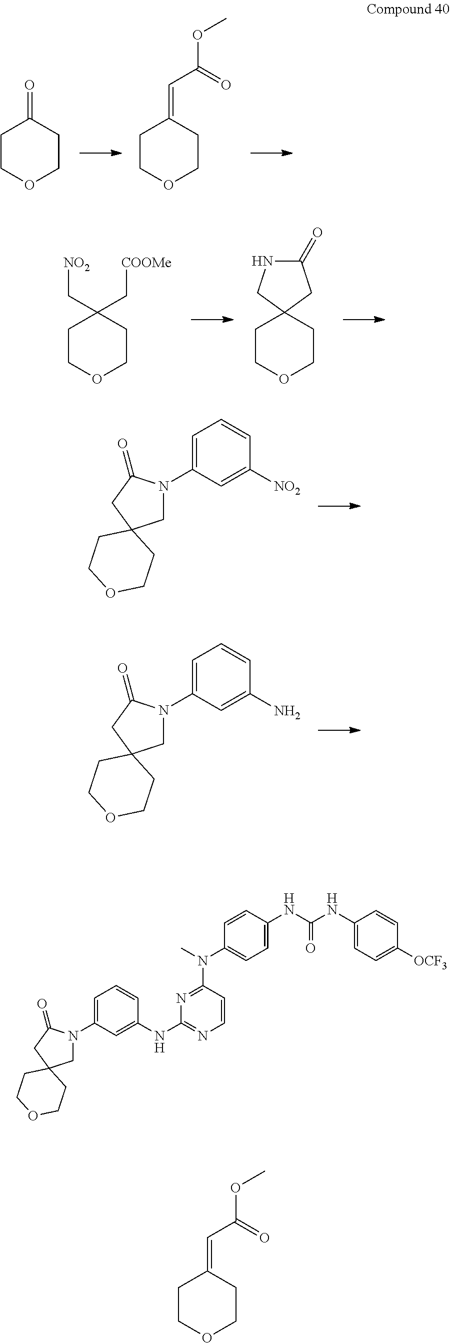

### Step 1: methyl 2-(dihydro-2H-pyran-4(3H)-ylidene)acetate

To a solution of dihydro-2H-pyran-4(3H)-one (10.0 g, 99.9 mmol) in toluene (300 mL) was added methyl 2-(triphenylphosphoranylidene)acetate (36.8 g, 109.9 mmol). The resulting mixture was stirred at 110° C. for 18 h. The reaction mixture was poured into EtOAc (100 mL), washed with water (3×100 mL), brine (100 mL), dried over anhydrous Na₂SO₄, filtered and concentrated under reduced pressure. The residue purified by column chromatography (pet.ether: EtOAc=10:1) to afford the titled product (11.5 g, 74%) as a yellow oil. LCMS (method B): 1.23 min [MH]⁺=157.1.

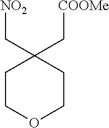 [SMILES: COC(=O)CC1(C[N+](=O)[O-])CCOCC1]

### Step 2: methyl 2-(tetrahydro-4-(nitromethyl)-2H-pyran-4-yl)acetate

To a solution of methyl 2-(dihydro-2H-pyran-4(3H)-ylidene)acetate (1.0 g, 6.40 mmol) in anhydrous THF (10 mL) was added a solution of TBAF in THF (1 M, 9.6 mL, 9.6 mmol), and nitromethane (0.68 mL, 12.80 mmol) dropwise. The resulting mixture was heated to 70° C. for 18 h. The reaction mixture was cooled to RT, diluted with EtOAc (15 mL), washed with HCl (1 M, 2×10mL) and brine (10 mL). The organic layer was dried over anhydrous Na₂SO₄, filtered, concentrated under reduced pressure. The residue was purified by column chromatography on silica gel (pet.ether:EtOAc=10:1) to afford a yellow solid (700 mg, 50%). LCMS (method B): 1.52 min [MH]⁺=218.1 ¹H NMR (400 MHz, d6-DMSO) δ ppm 1.59 (m, 4H) 2.61 (s, 2H) 3.61 (m. 7H) 4.78 (s, 2H)

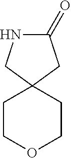 [SMILES: O=C1CC2(CCOCC2)CN1]

### Step 3: 8-oxa-2-azaspiro[4.5]decan-3-one

To a solution of methyl 2-(tetrahydro-4-(nitromethyl)-2H-pyran-4-yl)acetate (350 mg, 1.61 mmol) in MeOH (50 mL) was added Raney Ni (35 mg) and the mixture was stirred at 45° C. under H₂ atmosphere for 18 hours. The reaction mixture was filtered and the filtrate was concentrated under reduced pressure to afford a crude product (170 mg, 68%) as grey solid. LCMS (method B): 0.36 min [MNa]⁺=178.1 ¹H NMR (400 MHz, CDCl₃-d) δ ppm 1.67 (s, 4H) 2.27 (s, 2H) 3.23 (s. 2H) 3.67 (m, 4H) 6.17 (s, 1H).

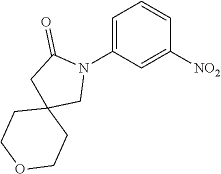 [SMILES: O=C1CC2(CCOCC2)CN1c1cccc([N+](=O)[O-])c1]

### Step 4: 2-(3-nitrophenyl)-8-oxa-2-azaspiro[4.5]decan-3-one

To a solution of 8-oxa-2-azaspiro[4.5]decan-3-one (170 mg, 1.09 mmol) in dioxane (20 mL) were added Pd₂(dba)₃(101 mg, 0.11 mmol), Xantphos (64 mg, 0.11 mmol), Cs₂CO₃ (710 mg, 2.18 mmol) under N₂ atmosphere. The resulting mixture was stirred at 100° C. for 16 hours. The reaction mixture was concentrated under reduced pressure and the residue was purified by column chromatography on silica gel (pet.ether:EtOAc=4:1 to 1:1) to afford the titled compound (100 mg, 33%) as a yellow solid LCMS (method B): 2.05 min [MH]⁺=277.1

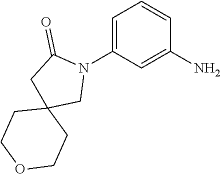 [SMILES: Nc1cccc(N2CC3(CCOCC3)CC2=O)c1]

### Step 5: 2-(3-aminophenyl)-8-oxa-2-azaspiro[4.5]decan-3-one

To a solution of 2-(3-nitrophenyl)-8-oxa-2-azaspiro[4.5]decan-3-one (100 mg, 0.36 mmol) in methanol (10 mL) were added zinc powder (234 mg, 3.60 mmol) and saturated aqueous ammonium chloride solution (2 mL). The resulting mixture was stirred at RT for 0.5 h. The reaction mixture was filtered and the filtrate was concentrated under reduced pressure. The residue was partitioned between aqueous sodium bicarbonate solution (2 mL), and EtOAc (2 mL) and the organic layer was extracted with EtOAc (3×5 mL). The combined organic layers were washed with brine (5 mL), dried over anhydrous NaSO₄, filtered and concentrated under reduced pressure to afford the titled compound (80 mg, 90%) as a yellow solid. LCMS (method B): 0.34 min [MH]⁺=247.1.

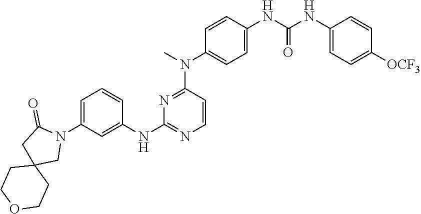 [SMILES: CN(c1ccc(NC(=O)Nc2ccc(OC(F)(F)F)cc2)cc1)c1ccnc(Nc2cccc(N3CC4(CCOCC4)CC3=O)c2)n1]

### Step 6: 1-(4-(methyl(2-((3-(3-oxo-8-oxa-2-azaspiro[4.5]decan-2-yl)phenyl) amino)pyrimidin-4-yl) amino)phenyl)-3-(4-(trifluoromethoxy)phenyl)urea

To a solution of 2-(3-aminophenyl)-8-oxa-2-azaspiro[4.5]decan-3-one (40 mg, 0.16 mmol) in isopropanol (15 mL) were added intermediate C (66 mg, 0.15 mmol) and conc. HCl (0.1 mL). The resulting mixture was stirred at 80° C. overnight. The reaction mixture was concentrated under reduced pressure and the residue was dissolved in NH₃.H₂O (3 mL), and extracted with DCM (3×10 mL). The combined organic layers were washed with brine (10 mL), dried over anhydrous NaSO₄, filtered and concentrated under reduced pressure. The residue was purified by prep-TLC (DCM:MeOH:NH₃.H₂O=30:1:0.3) to afford the titled compound (58 mg, 59%) as a white solid. LCMS (method B): 2.67 min [MH]⁺=648.3 ¹H NMR (400 MHz, CDCl₃) δ ppm 1.71 (m, 4H) 2.59 (s, 2H) 3.38 (s, 3H) 3.69 (m, 6H) 5.68 (d, J=6.4 Hz 1H) 7.18-7.30 (m 6H) 7.42 (d, J=8.0 Hz 1H) 7.55-7.57 (m, 4H) 7.85 (d, J=6.0 Hz 1H) 8.05 (s, 1H) 9.12-9.16 (m, 3H).

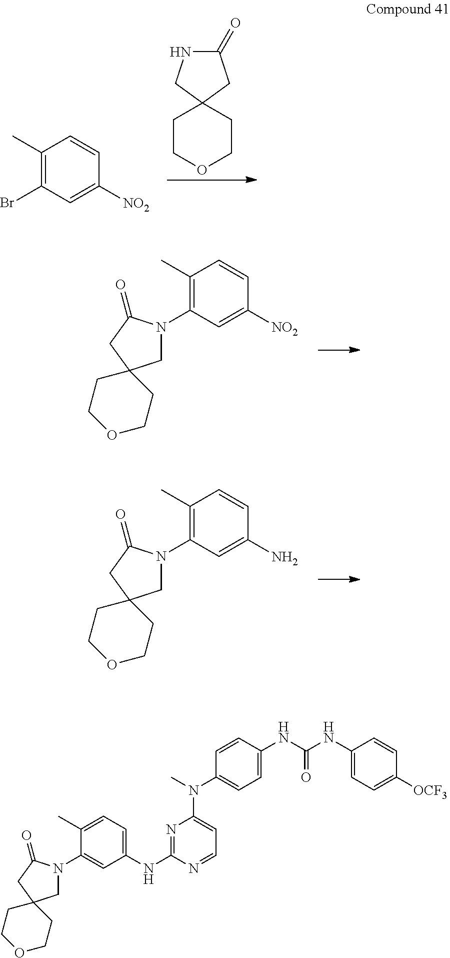 [SMILES: Cc1ccc([N+](=O)[O-])cc1Br ; O=C1CC2(CCOCC2)CN1 ; Cc1ccc([N+](=O)[O-])cc1N1CC2(CCOCC2)CC1=O ; Cc1ccc(N)cc1N1CC2(CCOCC2)CC1=O ; Cc1ccc(Nc2nccc(N(C)c3ccc(NC(=O)Nc4ccc(OC(F)(F)F)cc4)cc3)n2)cc1N1CC2(CCOCC2)CC1=O]

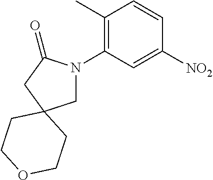 [SMILES: Cc1ccc([N+](=O)[O-])cc1N1CC2(CCOCC2)CC1=O]

### Step 1: 2-(2-methyl-5-nitrophenyl)-8-oxa-2-azaspiro[4.5]decan-3-one

To a solution of 2-bromo-1-methyl-4-nitrobenzene (500 mg, 2.31 mmol) in toluene (20 ml) were added 8-oxa-2-azaspiro[4.5]decan-3-one (717 mg, 4.63 mmol), K₂CO₃ (702 mg, 5.08 mmol), CuI (88 mg, 0.46 mmol), N1,N1-dimethylethane-1,2-diamine (0.86 mL, 9.24 mmol) under N₂ atmosphere. The resulting mixture was stirred at 110° C. overnight.The reaction mixture was poured into H₂O (10 mL) and extracted with EtOAc (3×10 mL). The combined organic layers were washed with brine (10 mL), dried over anhydrous Na₂SO₄, filtered and concentrated under reduced pressure. The residue was purified by column chromatography (DCM:MeOH=80:1) to afford crude product (80 mg) which was further purified by Prep-TLC (pet.ether:EtOAc=1:1) to afford a yellow solid (34 mg, 5%). LCMS (method B): 1.91 min [MH]⁺=291.1

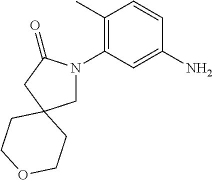 [SMILES: Cc1ccc(N)cc1N1CC2(CCOCC2)CC1=O]

### Step 2: 2-(5-amino-2-methylphenyl)-8-oxa-2-azaspiro[4.5]decan-3-one

To a solution of 2-(2-methyl-5-nitrophenyl)-8-oxa-2-azaspiro[4.5]decan-3-one (34 mg, 0.17 mmol) in MeOH (5 mL) were added Zn (76 mg,1.17 mmol) and a saturated aqueous NH₄Cl solution (1 mL). The resulting mixture was stirred at RT for 1.5 h. The reaction mixture was filtered and the filtrate was concentrated under reduced pressure. A saturated aqueous Na₂CO₃ solution (2 mL) and H₂O (3 mL) were added, and the solution was extracted with EtOAc (3×5 mL). The combined organic layers were washed with brine (5 mL), dried over anhydrous Na₂SO₄, filtered and concentrated under reduced pressure to afford a yellow solid (30 mg, 100%). LCMS (method B): 0.38 min [MH]⁺=261.2

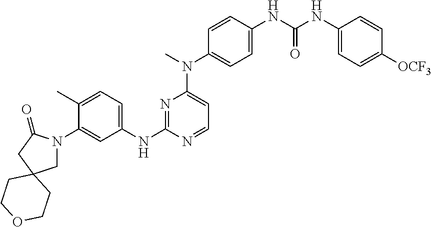 [SMILES: Cc1ccc(Nc2nccc(N(C)c3ccc(NC(=O)Nc4ccc(OC(F)(F)F)cc4)cc3)n2)cc1N1CC2(CCOCC2)CC1=O]

### Step 3: 1-(4-(methyl(2-((4-methyl-3-(3-oxo-8-oxa-2-azaspiro[4.5]decan-2-yl) phenyl)amino)pyrimidin-4-yl)amino)phenyl)-3-(4-(trifluoromethoxy)phenyl)urea

To a solution of 2-(5-amino-2-methylphenyl)-8-oxa-2-azaspiro[4.5]decan-3-one (30 mg, 0.12 mmol) in isopropanol (15 mL) were added intermediate C (53 mg, 0.12 mmol) and concentrated HCl (0.05 mL). The resulting mixture was stirred at 85° C. overnight. A saturated aqueous NaHCO₃ solution (5 mL) was added, followed by water (5 mL) and the mixture was extracted with DCM (3×10 mL). The combined organic layers were washed with brine (10 mL), dried over anhydrous Na₂SO₄, filtered and concentrated under reduced pressure. The residue was purified by Prep-TLC (DCM:MeOH:NH₃.H₂O=20:1:0.2) to afford the titled compound (30 mg, 40%) as a white solid.

LCMS (method B):2.67 min [MH]⁺=662.3 ¹HNMR (400 Mz, CDCl₃) δ ppm 1.78 (br s, 4H) 2.21 (s, 3H) 2.65 (s, 2H) 3.34 (s, 3H) 3.65 (s, 2H) 3.75 (br s, 4H) 5.59 (d, J=6.0 Hz 1H) 6.95 (m, 4H) 7.14 (m, 4H) 7.23 (m, 1H) 7.45 (d, J=8.8 Hz 2H) 7.63 (d, J=5.6 Hz 1H) 8.03 (s, 1H) 8.09 (br s, 1H) 8.40 (s, 1H).

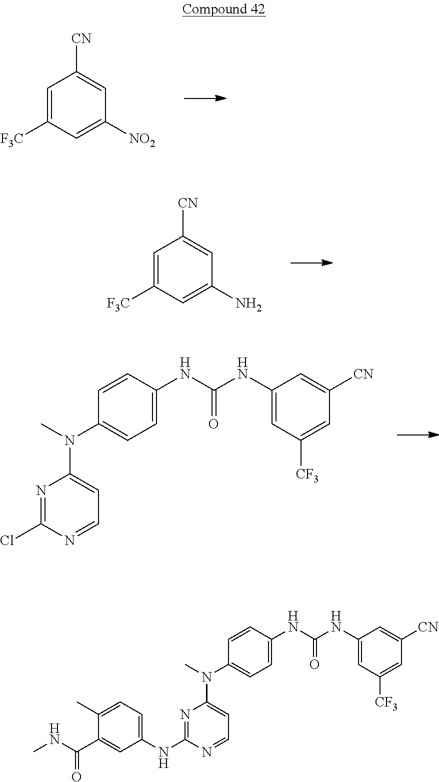 [SMILES: N#Cc1cc([N+](=O)[O-])cc(C(F)(F)F)c1 ; N#Cc1cc(N)cc(C(F)(F)F)c1 ; CN(c1ccc(NC(=O)Nc2cc(C#N)cc(C(F)(F)F)c2)cc1)c1ccnc(Cl)n1 ; CNC(=O)c1cc(Nc2nccc(N(C)c3ccc(NC(=O)Nc4cc(C#N)cc(C(F)(F)F)c4)cc3)n2)ccc1C]

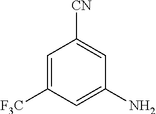 [SMILES: N#Cc1cc(N)cc(C(F)(F)F)c1]

### Step 1: 3-amino-5-(trifluoromethyl)benzonitrile

To a solution of 3-nitro-5-(trifluoromethyl)benzonitrile (432.0 mg, 2.0 mmol) in ethanol (5 mL) were added zinc (1.3 mg, 20 mmol), ammonium chloride aqueous solution (5 mL). The resultant mixture was stirred for 14 h at 60° C. The organic layer was filtered and the filtrate was concentrated under reduced pressure. The crude was partitioned between H₂O (5 ml) and EtOAc (5 mL) and the mixture was extracted with ethyl acetate (10 mL). The combined organic layers were dried over Na₂SO₄ and concentrated to give a residue which was purified by column chromatography (eluent: petroleum ether:ethyl acetate=2:1) to give a yellow solid (300.0 mg, 81%) LCMS (method B): 2.33 min [MH]⁺=187.1.

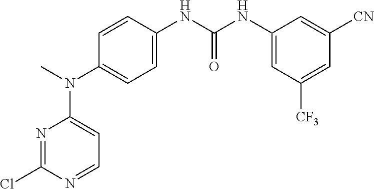 [SMILES: CN(c1ccc(NC(=O)Nc2cc(C#N)cc(C(F)(F)F)c2)cc1)c1ccnc(Cl)n1]

### Step 2: 1-(4-((2-chloropyrimidin-4-yl)(methyl)amino)phenyl)-3-(3-cyano-5-(trifluoromethyl)phenyl)urea

To a solution of 3-amino-5-(trifluoromethyl)benzonitrile (279.0 mg, 1.5 mmol) in DCM (5 mL) was added TEA (202.0 mg, 2.0 mmol) and BTC (97.9 mg, 0.34 mmol) at 0° C. for 15 min followed by N1-(2-chloropyrimidin-4-yl)-N1-methylbenzene-1,4-diamine (from step 1 intermediate C, 234.0 mg, 1.0 mmol). The resultant mixture was stirred at RT overnight. The reaction was quenched with the saturated NaHCO₃ aqueous solution (5 mL) and then the mixture was extracted with DCM (10 mL), dried over Na₂SO₄. and concentrated under reduced pressure. The residue was purified by column chromatography (eluent: petroleum ether:ethyl acetate=1:1) to give a yellow oil (108.0 mg 25%). LCMS (method B): 2.93 min [MH]+=447.1

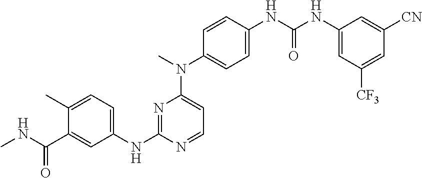 [SMILES: CNC(=O)c1cc(Nc2nccc(N(C)c3ccc(NC(=O)Nc4cc(C#N)cc(C(F)(F)F)c4)cc3)n2)ccc1C]

### Step 3: 5-(4-((4-(3-(2-cyano-5-(trifluoromethyl)phenyl)ureido)phenyl)(methyl) amino)pyrimidin-2-ylamino)-N,2-dimethylbenzamide

To a solution of 5-amino-N,2-dimethylbenzamide (28.0 mg, 0.17 mmol), 1-(4-((2-chloropyrimidin-4-yl)(methyl)amino)phenyl)-3-(3-cyano-5-(trifluoromethyl)phenyl)urea (50.0 mg,0.11 mmol) in isopropanol (10 mL) was added con HCl (3 drops) dropwise. The resultant mixture was stirred at 85° C. overnight. The reaction mixture was concentrated under reduced pressure and the residue was diluted with ethyl acetate (10 mL) and the mixture was washed with water (8 mL). The organic layer was separated, dried over Na₂SO₄ and concentrated under reduced pressure. The residue was purified by column chromatography (eluent: DCM:MeOH=15:1) to give the titled compound (10.0 mg, 16%) as a yellow solid.

LCMS (method B): 2.56 min [MH]⁺=575.3. ¹HNMR (400 MHz, CDCl3) δ 10.37 (s, 1H), 9.95 (s, 1H), 9.67 (s, 1H), 8.23 (s, 1H), 8.17 (d, J=4.5 Hz, 1H), 8.12 (s, 1H), 7.90 (s, 1H), 7.86 (d, J=6.6 Hz, 1H), 7.65 (d, J=8.7 Hz, 2H), 7.60 (s, 1H), 7.52 (s, 1H), 7.35 (d, J=8.7 Hz, 2H), 7.23 (s, 1H), 5.90 (br s, 1H), 3.48 (s, 3H), 2.75 (d, J=4.6 Hz, 3H), 2.29 (s, 3H).

Expression Constructs

cDNAs encoding mouse (residues 1-464) or human (residues 1-471) MLKL were synthesized to eliminate several restriction sites by silent substitutions (DNA2.0, CA). MLKL-encoding cDNAs were ligated into the doxycycline-inducible, puromycin selectable vector, pF TRE3G PGK puro, as described in Moujalled D M, et al. (2014), Cell Death Dis 5:e1086; Moujalled D M, et al. (2013) Cell Death Dis 4:e465; and Murphy J M, et al. (2013), Immunity 39(3):443-453. Sequences were verified by Sanger sequencing (Micromon DNA Sequencing Facility, VIC, Australia or by DNA2.0).

Lentiviral particles were produced by transfecting HEK293T cells seeded in 10 cm dishes with 1.2 μg of vector DNA together with two helper plasmids (0.8 μg of pVSVg and 2 μg of pCMV ΔR8.2) as described in Vince J E, et al. (2007), Cell 131(4):682-693. Viral supernatants were used to infect target cells with transfected cells selected for and maintained in 5 μg/ml puromycin.

Reagents and Antibodies

Recombinant hTNF-Fc was produced in-house as described in Bossen C, et aL (2006), The Journal of biological chemistry 281(20):13964-13971. Puromycin, Doxycycline and Necrostatin-1 were purchased from Sigma-Aldrich. The Smac mimetic, Compound A, has been described previously in Vince J E, et al. (2007), Cell 131(4):682-693. Q-VD-OPh was purchased from R&D systems.

1.2 Results of Assays

The compounds described herein were assayed as described above. The results of the assays are set out in Tables 1 and 2 below.

Assay 1: Screening Compounds for Inhibition of TSQ Induced Necroptosis, 96 Well Plate Format.

Cell Line ID: 0937 human histiocytic leukemia cell line.

Cell Concentration (cells/well): 35,000 per well in 120 μL of media, counted and plated immediately prior to addition of inhibitor and death stimuli. Final well volume of 150 μL after addition of compounds and death stimuli

Cell growth medium: HTRPMI (WEHI Media kitchen, contains L-Glutamine and penicillin, streptomycin)—supplemented with 7.4% v/v FCS (Gibco, Precision Plus. Lot #1221437)

Incubation time (hours): 48 hours following addition of compounds and death stimuli

Compound Concentrations—Log Titrations:

10000 nM, 5000 nM, 1000 nM, 500 nM, 100 nM, 50 nM, 10 nM, 5 nM, 1 nM, 0.5 nM, 0.1 nM

DMSO final concentration (% v/v): 1.2% in 10 μM well to 0.2% in the control well.

Compounds that are in the Death Stimulation Cocktail and Their Final Concentrations:

hTNF-Fc (100 ng/ml)—produced by standard procedures as shown in Bossen et al., J Biol Chem, 2006, 281(20), 13964-13971.

Compound A (500 nM)—Smac mimetic, Tetralogic

Q-VD-OPh (10 μM)—MP Biomedicals

Analysis:

Cells treated with PI staining (1 μg/ml) and analysed by flow cytometry

The results of the screening of the compounds described above are shown below in Table 1.

TABLE 1
Table showing the results of cell based assays performed under
assay 1 and binding data for compounds described above.

| | Inhibition of TSQ-induced | |
|---|---|---|
| | necroptosis | Off-target effect |
| | (cell based assay) | (cell based assay) |
| Compound | IC₅₀ (nM) | IC₅₀ (nM) |
| 1 | 53.0 | 4,679 |
| 2 | 29.5 | 2,110 |
| 3 | 93.1 | >10,000 |
| 4 | 154.0 | 7,410 |
| 5 | 30.0 | 3,110 |
| 7 | 51.8 | >10,000 |
| 8 | 75.4 | >10,000 |
| 9 | 97.3 | >10,000 |
| 10 | <1 | 506 |
| 11 | 7.5 | 1,347 |
| 12 | 10.1 | >10,000 |
| 13 | <1 | 654 |
| 14 | 8.9 | 1,632 |
| 15 | <1 | 103 |
| 16 | 10.8 | 1,230 |
| 17 | 38.3 | 5,669 |
| 18 | 514.0 | >10,000 |
| 19 | 1,160.0 | >10,000 |
| 20 | 504.0 | >10,000 |
| 21 | 476.0 | >10,000 |
| 22 | 427.0 | >10,000 |
| 23 | 202.0 | 6,379 |
| 24 | 8.9 | 1,237 |
| 25 | 9.2 | 2,278 |
| 26 | 19 | 1,250 |
| 27 | 325 | 2700 |
| 28 | 93 | 980 |
| 29 | 20 | 2,049 |
| 30 | 58 | 6,683 |
| 31 | 13 | NT |
| 32 | 40 | NT |
| 33 | 164 | 4,129 |
| 34 | 23 | 2416 |

NT—not tested

Assay 2: Screening Compounds for Inhibition of TSQ Induced Necroptosis, 96 Well Plate Format.

Cell Line ID: U937 human histiocytic leukemia cell line.

Cell Concentration (cells/well): Final cell density is 5000 cells per well.

Cell growth medium: HT-RPMI+7.4% FBS. Cells are cultured in Corning 150 cm² tissue culture flasks with vented caps at 37° C./5% CO₂.

Incubation time (hours): 48 hours following addition of compounds and death stimuli

Compound concentration: 100 nM

DMSO final concentration (% v/v): 0.3%.

Compounds that are in the Death Stimulation Cocktail and Their Final Concentrations:

hTNF-Fc (100 ng/ml)—produced by standard procedures as shown in Bossen et al., J Biol Chem, 2006, 281(20), 13964-13971.

Compound A (500 nM)—Smac mimetic, Tetralogic

Q-VD-OPh (10 μM)—MP Biomedicals

Analysis:

Data is loaded into Abase and normalised. 10 points titration curve are fitted with the 4 parameter logistic nonlinear regression model and the IC50 reported reflects the inflection point of the curve for curve fitting.

The results of the screening of the compounds described above are shown below in Table 2.

TABLE 2
Table showing the results of cell based assays performed under assay 2 and binding data for compounds described above.

| | Inhibition of TSQ- | |
|---|---|---|
| | induced necroptosis | Off-target effect |
| | (cell based assay) | (cell based assay) |
| Compound | IC₅₀ (U937, TSQ nM) | IC₅₀ (U937, μM) |
| 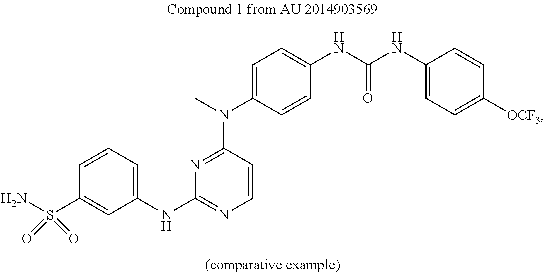 [SMILES: CN(c1ccc(NC(=O)Nc2ccc(OC(F)(F)F)cc2)cc1)c1ccnc(Nc2cccc(S(N)(=O)=O)c2)n1] | 75 | 1.5 |
| 6 | 29 | >1000 |
| 34 | 29 | 0.513 |
| 35 | 45 | 0.841 |
| 36 | 23 | 2.416 |
| 37 | 71 | 1.094 |
| 38 | 2.3 | 0.231 |
| 39 | 3.9 | 0.419 |
| 40 | 7.5 | >10 |
| 41 | 14 | >10 |
| 42 | 49 | >10 |

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A compound of Formula (I): 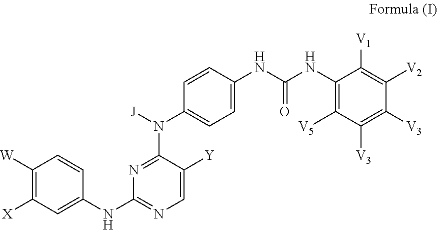
or a salt, solvate, or prodrug thereof
wherein
J is selected from hydrogen and methyl; and
Y is selected from hydrogen, methyl and halogen; and
W is selected from the group consisting of hydrogen, halogen, C₁-C₃ alkyl, C₁-C₃ haloalkyl, —OR¹ and (C₀-C₄ alkyl)C₃-C₇ heterocyclyl; and
X is selected from the group consisting of cyano, —OR¹, —(C₁-C₄ alkyl)NR³R⁴, C₃-C₇ cycloalkyl, (C₀-C₄ alkyl)C₃-C₇ heterocyclyl, aryl, heteroaryl, 4 to 7-membered lactam; and the group defined by -(A¹)ₘ-(A²)-(A³), wherein
A¹ is CH₂ and m is 0, 1, 2, or 3, or
A¹ is NR² and m is 0 or 1, or
A¹ is oxygen and m is 0 or 1, or
A¹ is CH₂NR² and m is 0 or 1;
A² is S(O)₂, S(O), or C(O); and
A³ is C₁-C₄ alkyl, C₁-C₄ alkoxy, C₁-C₄ hydroxyalkoxy, C₃-C₇ cycloalkyl, C₃-C₇ heterocyclyl, NR³R⁴, aryl, arylamino, aralkyl, aralkoxy, or heteroaryl;
R¹ is selected from the group consisting of hydrogen, C₁-C₄ alkyl, C₃-C₇ heterocyclyl, (C₀-C₄ alkyl)C₃-C₇ heterocyclyl and —NR³R⁴;
R², R³, and R⁴ are each independently selected from the group consisting of hydrogen, hydroxy, C₁-C₆ alkyl, C₁-C₆ alkoxy, aryloxy, aralkoxy, amino, C₁-C₆ alkylamino, arylamino, aralkylamino, C₁-C₄ alkyl, C₃-C₇ cycloalkyl, C₃-C₇heterocyclyl, —S(O)₂R⁵, and —C(O)R⁵; and
R⁵ is selected from C1-C4 alkyl, or C3-C7 cycloalkyl.
V₁, V₂, V₃, V₄ and V₅ are each independently selected from hydrogen and a group defined by —(X₄)_{z}—(X₅), wherein
X₄ is CH₂ where z is 0, 1, 2, 3, or 4, and
X₅ is selected from the group consisting of hydrogen, C₁-C₆ alkyl, C₁-C₆ haloalkyl, C₃-C₇ cycloalkyl, C₃-C₇ heterocyclyl, aryl, heteroaryl, C₁-C₆ alkoxy, C₁-C₆ haloalkoxy, hydroxy, aryloxy, aralkoxy, halo, —CN, —NR′R′, N(H)C(O)R″, N(H)C(O)OR″, N(H)C(O)NR′R′, N(H)S(O)₂R″, OR″, OC(O)RR″, C(O)R″, SR″, S(O)R′″, S(O)₂ R′″, and S(O)₂NR′R′, wherein
R′ is selected from the group consisting of hydrogen, C₁-C₆ alkyl, C₃-C₇ cycloalkyl, C₃-C₇ heterocyclyl, —OR¹, —SR¹, —S(O)₂R¹, —S(O)R¹, and C(O)R¹;
R″ is selected from the group consisting of hydrogen, C₁-C₆ alkyl, C₃-C₇ cycloalkyl, C₃-C₇ heterocyclyl, —NR³R⁴, —S(O)₂R¹, —S(O)R¹ and C(O)R¹; and
R′″ is selected from the group consisting of hydrogen, C₁-C₆ alkyl, C₃-C₇ cycloalkyl, C₃-C₇heterocyclyl, —OR¹ and —NR³R⁴;
provided that one or more of the following conditions is satisfied:
(i) Y is halo or methyl; and
(ii) X is selected from the group consisting of —CONR³R⁴, —(C₁-C₄ alkyl)-NR³R⁴, 4 to 7-membered lactam, heteroaryl, cyano, —OR¹ and 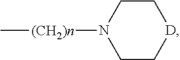
where D is O or NR⁶, wherein R⁶ is hydrogen or C₁-C₄ alkyl, and n is 1-4; and
(iii) V₁, V₃ and V₅ are hydrogen and V₂ and V₄ are each independently selected from the group consisting of halo, C₁-C₆ haloalkyl, C₁-C₆ alkyl and C₁-C₆ haloalkoxy.

2. A compound according to claim 1, wherein J is methyl.

3. A compound according to claim 1, wherein Y is halo.

4. (canceled)

5. A compound according to claim 1, wherein X is —CONR³R⁴.

6.-9. (canceled)

10. A compound according to claim 1, wherein X is heteroaryl.

11.-12. (canceled)

13. A compound according to claim 1, wherein X is unsubstituted 5 to 7-membered lactam, as illustrated below:

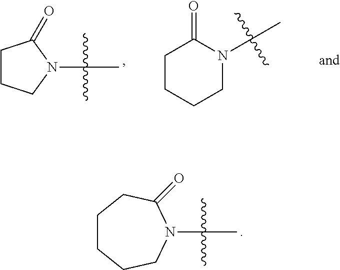 [SMILES: *N1CCCC1=O ; *N1CCCCC1=O ; *N1CCCCCC1=O]

14. (canceled)

15. A compound according to claim 1, wherein X is an oxo-substituted heterocyclyl group of the structure 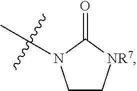
wherein R⁷ is hydrogen or C₁-C₄ alkyl.

16. A compound according to claim 1, wherein X is a spiro compound.

17. A compond according to claim 1, wherein X is —CH₂-morpholine or —CH₂-piperazine, optionally substituted with C₁-C₆ alkyl.

18. A compound according to claim 1, wherein W is methyl.

19. A compound according to claim 1, wherein W is morpholino.

20. A compound according to claim 1, wherein V₁, V₃ and V₅ are hydrogen and V₂ and V₄ are each independently selected from halo, C₁-C₆ haloalkyl, C₁-C₆ alkyl and C₁-C₆ haloalkoxy.

21. (canceled)

22. A compound according to claim 1, selected from the group of compounds consisting of:

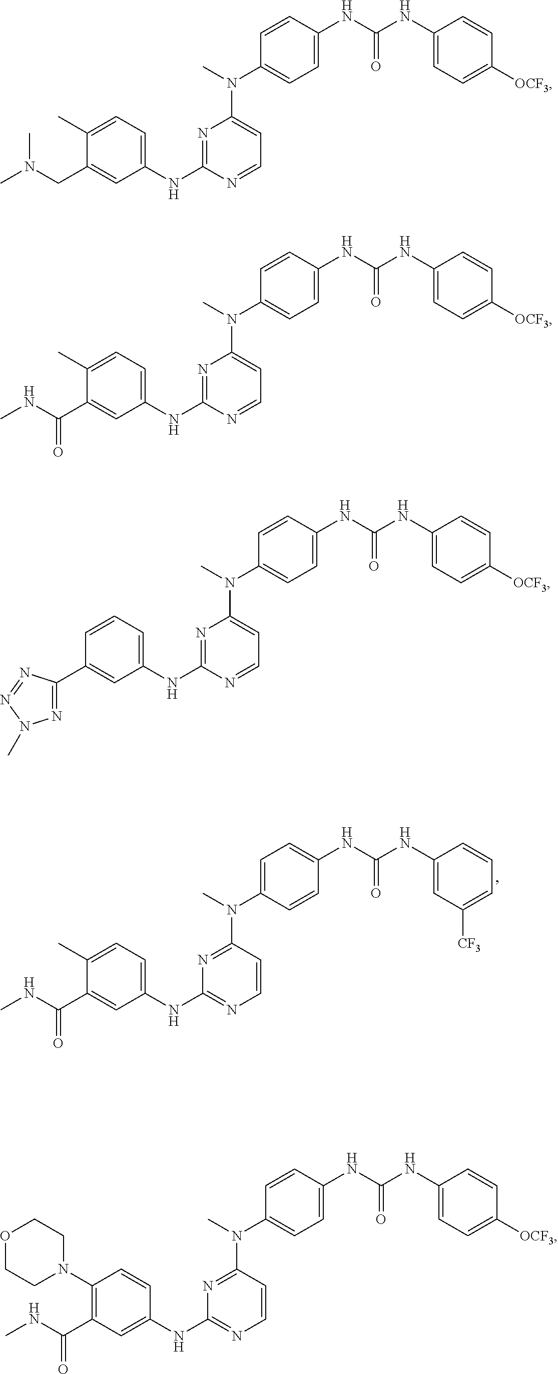 [SMILES: Cc1ccc(Nc2nccc(N(C)c3ccc(NC(=O)Nc4ccc(OC(F)(F)F)cc4)cc3)n2)cc1CN(C)C ; CNC(=O)c1cc(Nc2nccc(N(C)c3ccc(NC(=O)Nc4ccc(OC(F)(F)F)cc4)cc3)n2)ccc1C ; CN(c1ccc(NC(=O)Nc2ccc(OC(F)(F)F)cc2)cc1)c1ccnc(Nc2cccc(-c3nnn(C)n3)c2)n1 ; CNC(=O)c1cc(Nc2nccc(N(C)c3ccc(NC(=O)Nc4cccc(C(F)(F)F)c4)cc3)n2)ccc1C ; CNC(=O)c1cc(Nc2nccc(N(C)c3ccc(NC(=O)Nc4ccc(OC(F)(F)F)cc4)cc3)n2)ccc1N1CCOCC1]

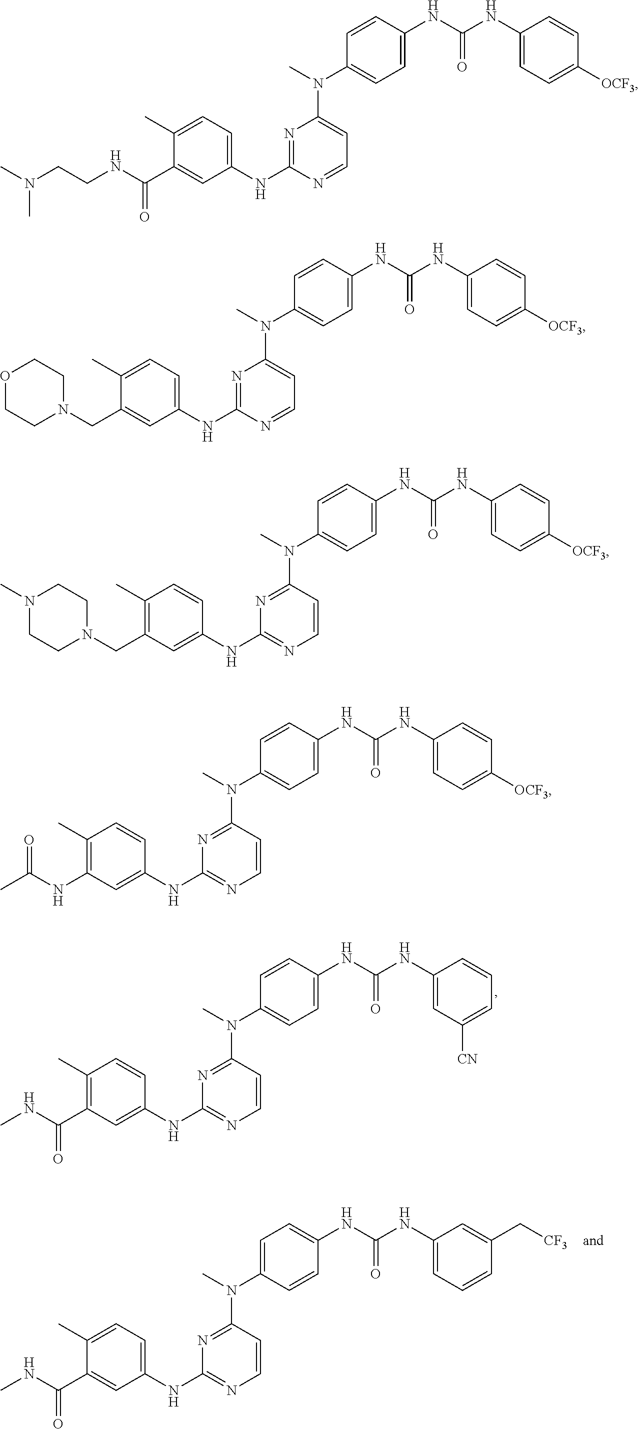

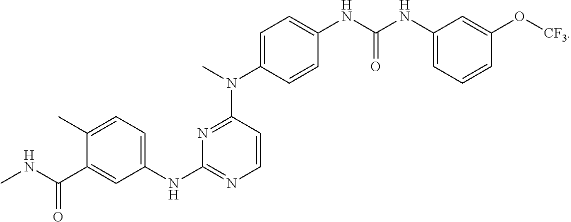 [SMILES: CNC(=O)c1cc(Nc2nccc(N(C)c3ccc(NC(=O)Nc4cccc(OC(F)(F)F)c4)cc3)n2)ccc1C]

23. A compound according to claim 1, selected from the group of compounds:

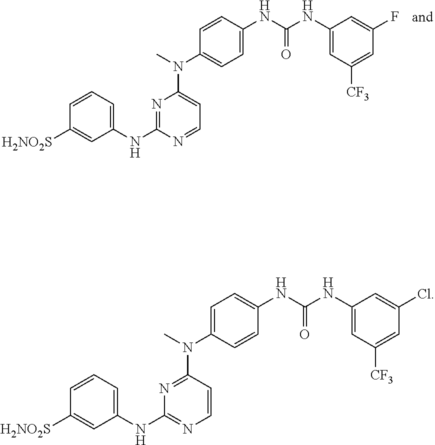 [SMILES: CN(c1ccc(NC(=O)Nc2cc(F)cc(C(F)(F)F)c2)cc1)c1ccnc(Nc2cccc(S(N)(=O)=O)c2)n1 ; "and" ; CN(c1ccc(NC(=O)Nc2cc(Cl)cc(C(F)(F)F)c2)cc1)c1ccnc(Nc2cccc(S(N)(=O)=O)c2)n1]

24. A compound according to claim 1, selected from the group of compounds:

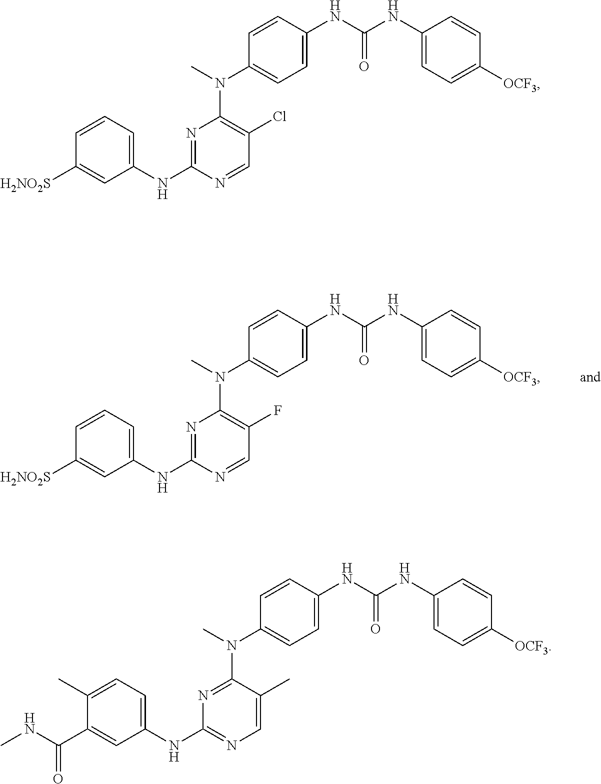 [SMILES: CN(c1ccc(NC(=O)Nc2ccc(OC(F)(F)F)cc2)cc1)c1nc(Nc2cccc(S(N)(=O)=O)c2)ncc1Cl ; CN(c1ccc(NC(=O)Nc2ccc(OC(F)(F)F)cc2)cc1)c1nc(Nc2cccc(S(N)(=O)=O)c2)ncc1F ; "and" ; CNC(=O)c1cc(Nc2ncc(C)c(N(C)c3ccc(NC(=O)Nc4ccc(OC(F)(F)F)cc4)cc3)n2)ccc1C]

25. A compound according to claim 1, selected from the group of compounds consisting of:

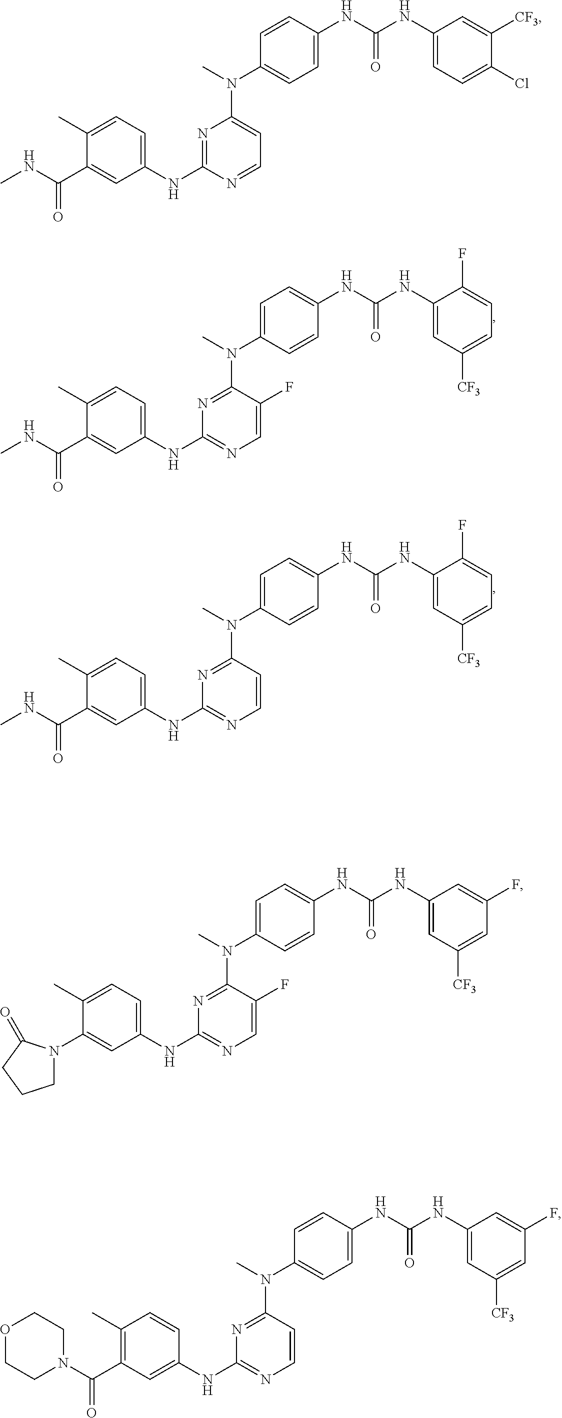 [SMILES: CNC(=O)c1cc(Nc2nccc(N(C)c3ccc(NC(=O)Nc4ccc(Cl)c(C(F)(F)F)c4)cc3)n2)ccc1C ; CNC(=O)c1cc(Nc2ncc(F)c(N(C)c3ccc(NC(=O)Nc4cc(C(F)(F)F)ccc4F)cc3)n2)ccc1C ; CNC(=O)c1cc(Nc2nccc(N(C)c3ccc(NC(=O)Nc4cc(C(F)(F)F)ccc4F)cc3)n2)ccc1C ; Cc1ccc(Nc2ncc(F)c(N(C)c3ccc(NC(=O)Nc4cc(F)cc(C(F)(F)F)c4)cc3)n2)cc1N1CCCC1=O ; Cc1ccc(Nc2nccc(N(C)c3ccc(NC(=O)Nc4cc(F)cc(C(F)(F)F)c4)cc3)n2)cc1C(=O)N1CCOCC1]

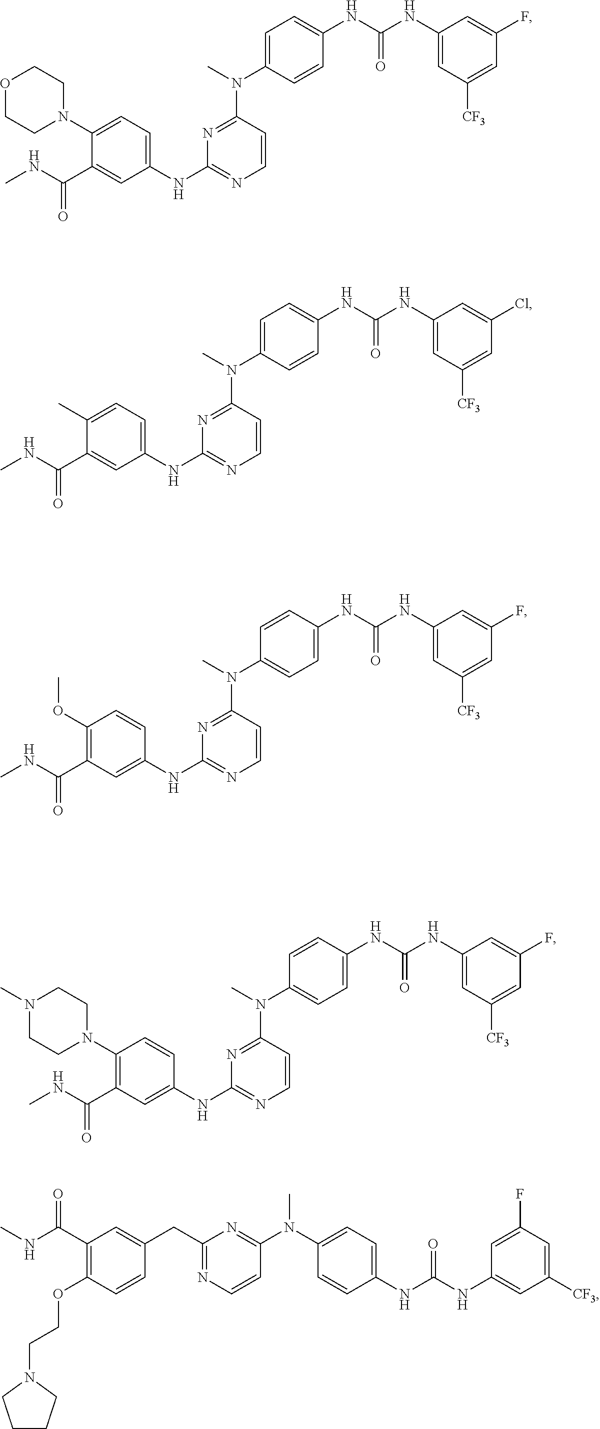 [SMILES: CNC(=O)c1cc(Nc2nccc(N(C)c3ccc(NC(=O)Nc4cc(F)cc(C(F)(F)F)c4)cc3)n2)ccc1N1CCOCC1 ; CNC(=O)c1cc(Nc2nccc(N(C)c3ccc(NC(=O)Nc4cc(Cl)cc(C(F)(F)F)c4)cc3)n2)ccc1C ; CNC(=O)c1cc(Nc2nccc(N(C)c3ccc(NC(=O)Nc4cc(F)cc(C(F)(F)F)c4)cc3)n2)ccc1OC ; CNC(=O)c1cc(Nc2nccc(N(C)c3ccc(NC(=O)Nc4cc(F)cc(C(F)(F)F)c4)cc3)n2)ccc1N1CCN(C)CC1 ; CNC(=O)c1cc(Cc2nccc(N(C)c3ccc(NC(=O)Nc4cc(F)cc(C(F)(F)F)c4)cc3)n2)ccc1OCCN1CCCC1]

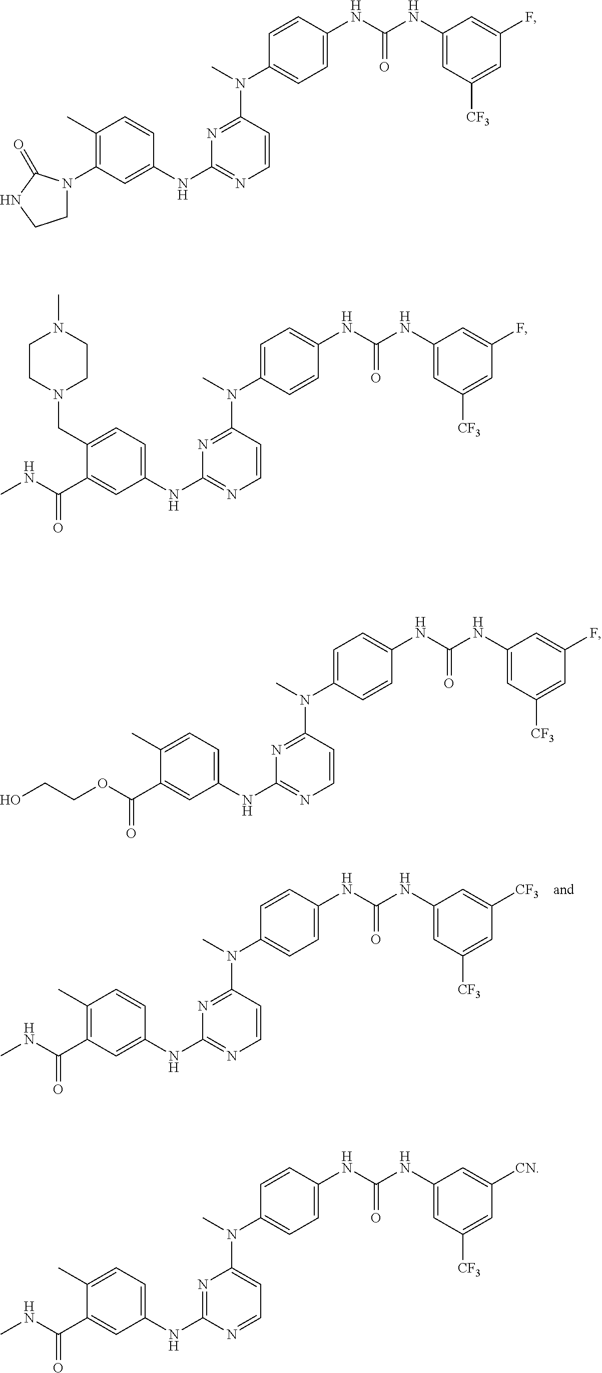 [SMILES: Cc1ccc(Nc2nccc(N(C)c3ccc(NC(=O)Nc4cc(F)cc(C(F)(F)F)c4)cc3)n2)cc1N1CCNC1=O ; CNC(=O)c1cc(Nc2nccc(N(C)c3ccc(NC(=O)Nc4cc(F)cc(C(F)(F)F)c4)cc3)n2)ccc1CN1CCN(C)CC1 ; Cc1ccc(Nc2nccc(N(C)c3ccc(NC(=O)Nc4cc(F)cc(C(F)(F)F)c4)cc3)n2)cc1C(=O)OCCO ; CNC(=O)c1cc(Nc2nccc(N(C)c3ccc(NC(=O)Nc4cc(C(F)(F)F)cc(C(F)(F)F)c4)cc3)n2)ccc1C ; "and" ; CNC(=O)c1cc(Nc2nccc(N(C)c3ccc(NC(=O)Nc4cc(C#N)cc(C(F)(F)F)c4)cc3)n2)ccc1C]

26. A compound according to claim 1, selected from the group of compounds consisting of:

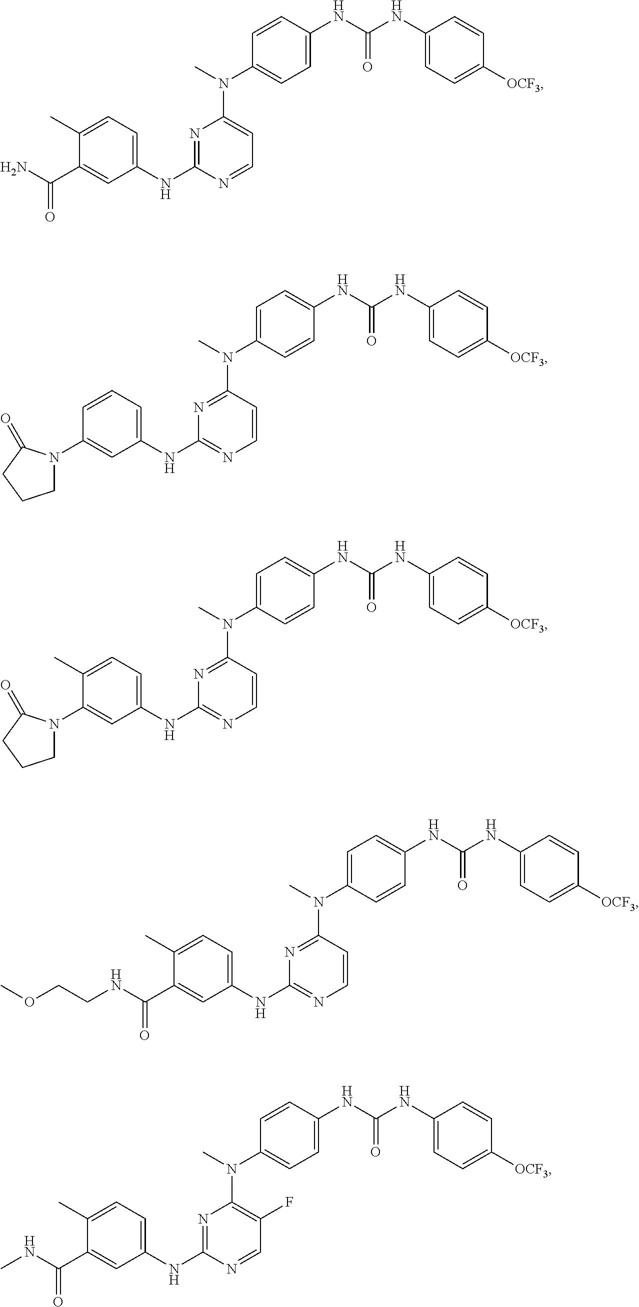 [SMILES: Cc1ccc(Nc2nccc(N(C)c3ccc(NC(=O)Nc4ccc(OC(F)(F)F)cc4)cc3)n2)cc1C(N)=O ; CN(c1ccc(NC(=O)Nc2ccc(OC(F)(F)F)cc2)cc1)c1ccnc(Nc2cccc(N3CCCC3=O)c2)n1 ; Cc1ccc(Nc2nccc(N(C)c3ccc(NC(=O)Nc4ccc(OC(F)(F)F)cc4)cc3)n2)cc1N1CCCC1=O ; COCCNC(=O)c1cc(Nc2nccc(N(C)c3ccc(NC(=O)Nc4ccc(OC(F)(F)F)cc4)cc3)n2)ccc1C ; CNC(=O)c1cc(Nc2ncc(F)c(N(C)c3ccc(NC(=O)Nc4ccc(OC(F)(F)F)cc4)cc3)n2)ccc1C]

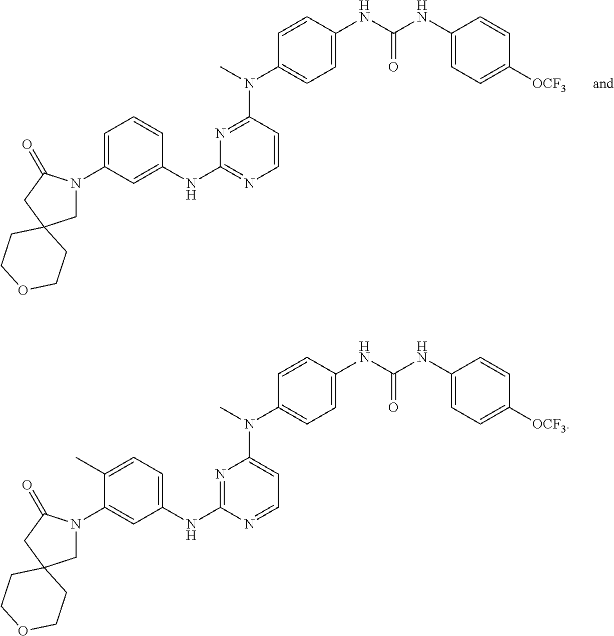 [SMILES: CN(c1ccc(NC(=O)Nc2ccc(OC(F)(F)F)cc2)cc1)c1ccnc(Nc2cccc(N3CC4(CCOCC4)CC3=O)c2)n1 ; "and" ; Cc1ccc(Nc2nccc(N(C)c3ccc(NC(=O)Nc4ccc(OC(F)(F)F)cc4)cc3)n2)cc1N1CC2(CCOCC2)CC1=O]

27. A compound according to claim 1, selected from the group of compounds consisting of:

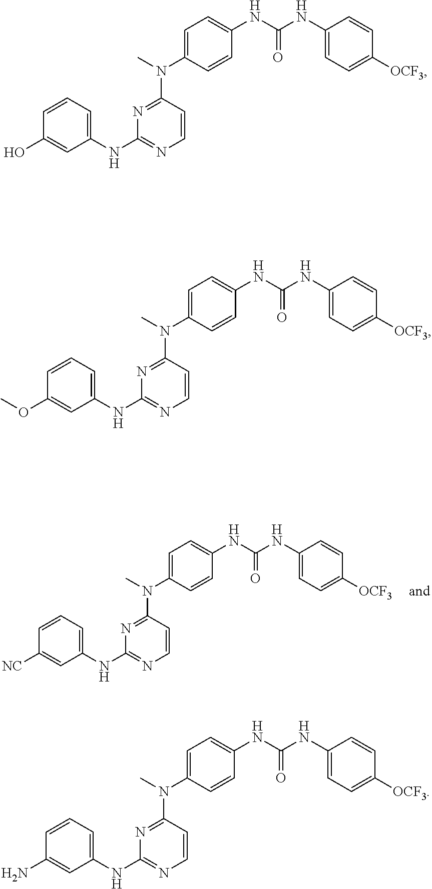 [SMILES: CN(c1ccc(NC(=O)Nc2ccc(OC(F)(F)F)cc2)cc1)c1ccnc(Nc2cccc(O)c2)n1 ; COc1cccc(Nc2nccc(N(C)c3ccc(NC(=O)Nc4ccc(OC(F)(F)F)cc4)cc3)n2)c1 ; CN(c1ccc(NC(=O)Nc2ccc(OC(F)(F)F)cc2)cc1)c1ccnc(Nc2cccc(C#N)c2)n1 ; "and" ; CN(c1ccc(NC(=O)Nc2ccc(OC(F)(F)F)cc2)cc1)c1ccnc(Nc2cccc(N)c2)n1]

28. A composition comprising a compound according to claim 1 or a salt, solvate, or prodrug thereof, and a pharmaceutically acceptable excipient.

29. A method for inhibiting necroptosis in a subject in need thereof, the method comprising administering a therapeutically effective amount of a compound according to claim 1 or a salt, solvate, or prodrug thereof.

30.-35. (canceled)
